(19) United States
(12) Patent Application Publication
(10) Pub. No.: US 2022/0332849 A1
(43) Pub. Date: Oct. 20, 2022

(54) Stabilized Formulations Containing Anti-MUC16 x Anti-CD3 Bispecific Antibodies

(71) Applicant: Regeneron Pharmaceuticals, Inc., Tarrytown, NY (US)

(72) Inventors: Douglas Kamen, Poughquag, NY (US); Teng-Chieh Yang, New Haven, CT (US); Xiaobin Xu, Old Greenwich, CT (US); Haibo Qiu, Hartsdale, NY (US); Kenneth Graham, Pleasant Valley, NY (US)

(21) Appl. No.: 17/711,800

(22) Filed: Apr. 1, 2022

Related U.S. Application Data
(60) Provisional application No. 63/170,320, filed on Apr. 2, 2021.
(60) Provisional application No. 63/313,927, filed on Feb. 25, 2022.

(51) Int. Cl. C07K 16/46; C07K 16/28

(52) U.S. Cl. CPC C07K 16/468; C07K 16/2809; A61K 2039/505

(57) ABSTRACT

The present invention provides stable liquid pharmaceutical formulations comprising a human bispecific antibody that specifically binds to human MUC16 and human CD3. In certain embodiments, the formulations contain, in addition to the bispecific antibody, a buffer, a surfactant, and a sugar. The pharmaceutical formulations of the present invention exhibit a substantial degree of antibody stability upon stress and storage.

### CROSS-REFERENCE TO RELATED APPLICATIONS

This application claims the benefit under 35 USC § 119(e) of U.S. Provisional Application No. 63/170,320, filed Apr. 2, 2021; and 63/313,927, filed Feb. 25, 2022, each of which is incorporated herein by reference in its entirety for all purposes.

### REFERENCE TO A SEQUENCE LISTING

This application incorporates by reference the Sequence Listing submitted in Computer Readable Form as file 10820US01-Sequence.txt, created on Apr. 1, 2022 and containing 25,436 bytes.

### FIELD OF THE INVENTION

The present invention relates to the field of therapeutic antibody formulations. More specifically, the present invention relates to the field of pharmaceutical formulations comprising a human bispecific antibody that specifically binds to human MUC16 and human CD3.

### BACKGROUND

Therapeutic macromolecules (e.g., antibodies) must be formulated in a manner that not only makes the molecules suitable for administration to patients, but also maintains their stability during storage and subsequent use. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the concentration of antibody that can be accommodated by a given formulation, and the visual quality or appeal of the formulation. Thus, when formulating a therapeutic antibody, great care must be taken to arrive at a formulation that remains stable, contains an adequate concentration of antibody, and possesses other properties which enable the formulation to be conveniently administered to patients.

Mucin 16 (MUC16), also known as cancer antigen 125, carcinoma antigen 125, carbohydrate antigen 125, or CA-125, is a single transmembrane domain highly glycosylated integral membrane glycoprotein that is highly expressed in ovarian cancer. CD3 is a homodimeric or heterodimeric antigen expressed on T cells in association with the T cell receptor complex (TCR) and is required for T cell activation.

Bispecific antibodies to human MUC16 and human CD3 are one example of therapeutically relevant macromolecules that require proper formulation. Such antibodies are clinically useful for, e.g., the treatment of cancer (e.g., MUC16-expressing cancers, ovarian cancer, breast cancer, pancreatic cancer, and non-small-cell lung cancer).

Although anti-MUC16×anti-CD3 bispecific antibodies are known in the art (see, e.g., WO 2018/067331), there remains a need for pharmaceutical formulations comprising anti-MUC16×anti-CD3 bispecific antibodies that are sufficiently stable and suitable for administration to patients.

### BRIEF SUMMARY OF THE INVENTION

Stable liquid pharmaceutical formulations comprising a bispecific anti-MUC16×anti-CD3 antibody and one or more excipients, as well as kits, unit dosage forms, and containers comprising such formulations and uses thereof, are provided.

In one aspect, the present invention provides a stable liquid pharmaceutical formulation comprising: (a) a bispecific antibody comprising a first antigen-binding domain that binds specifically to human MUC16 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15; (b) a buffer comprising sodium acetate; (c) an organic co-solvent comprising polysorbate; and (d) a stabilizer comprising a sugar; wherein the formulation has a pH of 5.0±0.5.

In some cases, the antibody concentration is from 1 mg/ml±0.1 mg/ml to 200 mg/ml±20 mg/ml. In some cases, the antibody concentration is from 5 mg/ml±0.5 mg/ml to 50 mg/ml±5 mg/ml. In some cases, the antibody concentration is 5 mg/ml±0.5 mg/ml. In some cases, the antibody concentration is 50 mg/ml±5 mg/ml. In some cases, the antibody concentration is 150 mg/ml±15 mg/ml.

In some cases, the acetate buffer concentration is from 10 mM±1 mM to 50 mM±5 mM. In some cases, the acetate buffer concentration is from 25 mM±2.5 mM to 35 mM±3.5 mM. In some cases, the acetate buffer concentration is 30 mM±3 mM.

In some cases, the polysorbate concentration is from 0.01%±0.005% to 0.5%±0.05% w/v. In some cases, the polysorbate concentration is from 0.1%±0.05% to 0.3%±0.03% w/v. In some cases, the polysorbate concentration is 0.2%±0.02% w/v. In some cases, the polysorbate concentration is 0.05%±0.01% w/v. In some embodiments, the polysorbate is polysorbate 20.

In some embodiments, the sugar is sucrose. In some cases, the sucrose concentration is from 5%±1% to 20%±4% w/v. In some cases, the sucrose concentration is from 7%±0.5% to 12%±0.5% w/v. In some cases, the sucrose concentration is 10%±1% w/v. In some cases, the sucrose concentration is 7%±0.7% w/v. In some cases, the sucrose concentration is 8%±0.8% w/v.

In some embodiments, the pharmaceutical formulation comprises: (a) 5 mg/ml±0.5 mg/ml antibody, (b) from 25 mM±2 mM to 35 mM±2 mM acetate buffer, (c) from 0.1%±0.05% to 0.3%±0.05% w/v polysorbate, and (d) from 5%±1% to 15%±3% w/v sucrose, at pH 5.0±0.5.

In some embodiments, the pharmaceutical formulation comprises: (a) 5 mg/ml±0.5 mg/ml antibody, (b) 30 mM±1 mM acetate buffer, (c) 0.2%±0.02% w/v polysorbate, and (d) 10%±1% w/v sucrose, at pH 5.0±0.3.

In some embodiments, the pharmaceutical formulation comprises: (a) 50 mg/ml±5 mg/ml antibody, (b) from 25 mM±2 mM to 35 mM±2 mM acetate buffer, (c) from 0.1%±0.05% to 0.3%±0.05% w/v polysorbate, and (d) from 5%±1% to 15%±3% w/v sucrose, at pH 5.0±0.5.

In some embodiments, the pharmaceutical formulation comprises: (a) 50 mg/ml±0.5 mg/ml antibody, (b) 30 mM±1 mM acetate buffer, (c) 0.2%±0.02% w/v polysorbate, and (d) 10%±1% w/v sucrose, at pH 5.0±0.3.

In any of these embodiments, the polysorbate may be polysorbate 20.

In any of the various embodiments discussed above, the formulation contains no more than 2.5% high molecular weight (HMW) species after 12 months or 24 months of storage at 5° C., as determined by size exclusion ultra performance liquid chromatography (SE-UPLC). In some cases, the formulation contains no more than 3.5% high molecular weight (HMW) species after 6 months of storage at 25° C. and 60% relative humidity, as determined by SE-UPLC. In some cases, the formulation contains no more than 1.5% high molecular weight (HMW) species after 12 months of storage at −30° C., or no more than 2.0% HMW species after 24 months of storage at −30° C., as determined by SE-UPLC. In some cases, the formulation contains no more than 1.5% high molecular weight (HMW) species after 12 months of storage at −80° C., or no more than 2.0% HMW species after 24 months of storage at −30° C., as determined by SE-UPLC.

In one aspect, the present invention provides a stable liquid pharmaceutical formulation reconstituted from a lyophilisate, comprising: (a) a bispecific antibody at a concentration of from 1 mg/ml to 30 mg/ml, wherein the bispecific antibody comprises a first antigen-binding domain that binds specifically to human MUC16 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15; (b) a buffer comprising histidine; (c) an organic co-solvent comprising polysorbate; and (d) a stabilizer comprising a sugar; wherein the formulation has a pH of 6.0±0.5.

In some cases, the antibody concentration is 2 mg/ml±0.5 mg/ml. In some cases, the antibody concentration is 20 mg/ml±2 mg/ml. In some cases, the histidine buffer concentration is from 5 mM±1 mM to 15 mM±1 mM. In some cases, the histidine buffer concentration is 10 mM±1 mM. In some cases, the polysorbate concentration is from 0.01% to 0.1% w/v. In some cases, the polysorbate concentration is 0.05%±0.01% w/v. In some embodiments, the polysorbate is polysorbate 20. In some embodiments, the sugar is sucrose. In some cases, the sucrose concentration is from 8%±0.5% to 12%±0.5% w/v. In some cases, the sucrose concentration is 10%±1% w/v.

In any of the embodiments of this aspect of the invention: (a) at least 95% of the antibody has native conformation after 12 months, after 18 months, after 24 months, or after 36 months of storage at 5° C.; (b) at least 95% of the antibody has native conformation after 6 months of storage at 25° C. and 60% relative humidity; (c) at least 95% of the antibody has native conformation after 3 months of storage at 37° C.; (d) the formulation contains no more than 1% high molecular weight (HMW) species after 12 months, after 18 months, after 24 months, or after 36 months of storage at 5° C.; (e) the formulation contains no more than 1% HMW species after 6 months of storage at 25° C. and 60% relative humidity; or (f) the formulation contains no more than 1% HMW species after 3 months of storage at 37° C.; as determined by SE-UPLC.

In one aspect, the present invention provides a stable liquid pharmaceutical formulation comprising: (a) a bispecific antibody at a concentration of from 100 mg/ml to 200 mg/ml, wherein the bispecific antibody comprises a first antigen-binding domain that binds specifically to human MUC16 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15; (b) a buffer comprising acetate; (c) a stabilizer comprising a sugar; and (d) a surfactant comprising polysorbate, wherein the formulation has a pH of 5.0±0.5.

In some cases, the antibody concentration is from 125 mg/ml to 175 mg/ml. In some cases, the antibody concentration is 150 mg/ml±10 mg/ml. In some embodiments, the sugar is sucrose. In some cases, the sucrose concentration is from 4% to 12% w/v. In some cases, the sucrose concentration is 8% w/v±1% w/v. In some cases, the acetate buffer concentration is from 25 mM to 35 mM. In some cases, the acetate buffer concentration is 30 mM±1 mM. In some cases, the polysorbate is polysorbate 20. In some cases, the polysorbate 20 concentration is from 0.01% w/v to 0.1% w/v. the polysorbate 20 concentration is 0.05% w/v±0.01% w/v.

In any of the embodiments of this aspect of the invention: (a) the formulation contains no more than 2.5% high molecular weight (HMW) species after 12 months or after 24 months of storage at −30° C. or −80 C; (b) the formulation contains no more than 4% HMW species after 6 months of storage at 5° C.; or (c) the formulation contains no more than 6% HMW species after 6 months of storage at 25° C. and 60% relative humidity; as determined by SE-UPLC.

In any of the various embodiments discussed above, or herein, the formulation contains no more than 40%, no more than 39%, no more than 38%, nor more than 37%, nor more than 36%, or no more than 35% of a glycated species variant, wherein the glycated species variant comprises glycation at residue 98 of SEQ ID NO: 1 or SEQ ID NO: 4, or residue 2 of SEQ ID NO: 9.

In any of the various embodiments discussed above, or herein, the first antigen-binding domain comprises a HCVR with at least 90% identity to the amino acid sequence of SEQ ID NO: 4 and a LCVR with at least 90% identity to the amino acid sequence of SEQ ID NO: 6, and the second antigen-binding domain comprises a HCVR with at least 90% identity to the amino acid sequence of SEQ ID NO: 5 and a LCVR with at least 90% identity to the amino acid sequence of SEQ ID NO: 6.

In any of the various embodiments discussed above, or herein, the first antigen-binding domain comprises a HCVR with at least 95% identity to the amino acid sequence of SEQ ID NO: 4 and a LCVR with at least 95% identity to the amino acid sequence of SEQ ID NO: 6, and the second antigen-binding domain comprises a HCVR with at least 95% identity to the amino acid sequence of SEQ ID NO: 5 and a LCVR with at least 95% identity to the amino acid sequence of SEQ ID NO: 6.

In any of the various embodiments discussed above, or herein, the first antigen-binding domain comprises a HCVR with at least 99% identity to the amino acid sequence of SEQ ID NO: 4 and a LCVR with at least 99% identity to the amino acid sequence of SEQ ID NO: 6, and the second antigen-binding domain comprises a HCVR with at least 99% identity to the amino acid sequence of SEQ ID NO: 5 and a LCVR with at least 99% identity to the amino acid sequence of SEQ ID NO: 6.

In any of the various embodiments discussed above, or herein, the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6.

In one aspect, the present invention provides a stable pharmaceutical formulation comprising: (a) 5 mg/ml±0.5 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 30 mM±1 mM sodium acetate buffer, pH 5.0±0.2, (c) 0.2%±0.02% w/v polysorbate 20, and (d) 10%±1% w/v sucrose.

In one aspect, the present invention provides a stable pharmaceutical formulation comprising: (a) 50 mg/ml±5 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 30 mM±1 mM sodium acetate buffer, pH 5.0±0.2, (c) 0.2%±0.02% w/v polysorbate 20, and (d) 10%±1% w/v sucrose.

In one aspect, the present invention provides a stable pharmaceutical formulation comprising: (a) 150 mg/ml±15 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 30 mM±1 mM sodium acetate buffer, pH 5.0±0.2, (c) 0.05%±0.01% w/v polysorbate 20, and (d) 8%±1% w/v sucrose.

In any of the various embodiments discussed above, or herein, the antibody comprises a human IgG heavy chain constant region attached, respectively, to the HCVR of each of the first antigen-binding domain and the second antigen-binding domain. In some cases, the heavy chain constant region is of isotype IgG1. In some cases, the heavy chain constant region is of isotype IgG4.

In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In any of the various embodiments discussed above, or herein, the antibody comprises a heavy chain constant region comprising an amino acid sequence selected from the group consisting of SEQ ID NO: 16, SEQ ID NO: 17, SEQ ID NO: 18, and SEQ ID NO: 19. In some embodiments, the antibody comprises a heavy chain constant region comprising the amino acid sequence of SEQ ID NO: 16 and a heavy chain constant region comprising the amino acid sequence of SEQ ID NO: 17. In some embodiments, the antibody comprises a heavy chain constant region comprising the amino acid sequence of SEQ ID NO: 18 and a heavy chain constant region comprising the amino acid sequence of SEQ ID NO: 19.

In any of the various embodiments discussed above, or herein, the antibody comprises a first heavy chain containing the HCVR of the first antigen-binding domain and a second heavy chain containing the HCVR of the second antigen-binding domain, wherein the first heavy chain comprises residues 1-442 of the amino acid sequence of SEQ ID NO: 1 and the second heavy chain comprises residues 1-449 of the amino acid sequence of SEQ ID NO: 2. In some embodiments, the antibody comprises a common light chain containing the LCVR of the first and second antigen-binding domains, wherein the common light chain comprises the amino acid sequence of SEQ ID NO: 3.

In any of the various embodiments discussed above or herein, the pharmaceutical formulation may be deemed stable by measuring the percentage change in “glycated species,” wherein the percentage change in glycated species is: (i) no more than 1.5% after 6 months of storage at 5° C.; (ii) no more than 3% after 12 months of storage at 5° C.; (iii) no more than 1.5% after 12 months, after 18 months, or after 24 months of storage at −30° C.; or no more than 1% after 12 months, after 18 months, or after 24 months of storage at −80° C., as determined by cation exchange ultra performance liquid chromatography (CEX-UPLC), and/or by liquid chromatography-mass spectrometry (LC-MS).

In one aspect, the present invention provides a pharmaceutical composition, wherein the composition comprises the pharmaceutical formulation as discussed above or herein, and the composition is contained in a container.

In some embodiments, the container is a vial. In some cases, the vial is a 2 ml, 5 ml or 10 ml Type 1 clear glass vial. In some embodiments, the container is a syringe. In some cases, the syringe is low-tungsten glass. In some embodiments, the container is a prefilled syringe. In some embodiments, the pharmaceutical composition is contained in an autoinjector.

In one aspect, the present invention provides a kit comprising (i) a container containing a composition comprising the pharmaceutical formulation as discussed above or herein, and instructions for use of the composition.

In some embodiments, the container is a glass vial. In some embodiments, the container is a prefilled syringe. In some embodiments, the container is an autoinjector.

In some embodiments, the instructions recite subcutaneous administration of the composition. In some embodiments, the instructions recite intravenous administration of the composition.

In one aspect, the present invention provides a unit dosage form comprising the pharmaceutical formulation discussed above or herein, wherein the antibody is present in an amount of from 0.1 mg to 500 mg. In some cases, the antibody is present in an amount of from 5±1 mg to 50±5 mg. In some cases, the antibody is present in an amount of from 10±1 mg to 200±20 mg. In some embodiments, the antibody is present in an amount of 4 mg, 5 mg, 10 mg, 12.5 mg, 40 mg, 50 mg, 150 mg, or 180 mg.

In some embodiments, the unit dosage form is a glass vial, a prefilled syringe, or an autoinjector.

In one aspect, the present invention provides a container containing a composition comprising the pharmaceutical formulation as discussed above or herein. In various embodiments, the container is a glass vial, a prefilled syringe, or an autoinjector.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation).

Other embodiments will become apparent from a review of the ensuing detailed description.

### BRIEF DESCRIPTION OF THE DRAWINGS

FIG. 1 illustrates the relationship between relative potentcy of mAb1 and the level of glycation of HCDR3-Lys98. Glycation levels were generated by purification of glycated and non-glycated mAb1 by preparative cation exchange chromatography and mixing of both species at different ratios. The potency data indicate that the potency is dependent on the level of glycation.

FIGS. 2A, 2B, 2C and 2D illustrate the effect of pH on the level of glycated and high molecular weight (HMW) species for mAb1 formulated in histidine buffer. The formulations included 2 mg/ml mAb1 in 10 mM histidine, 10% w/v sucrose, and 0.05% w/v polysorbate 20 with different pH values, and were incubated at 5° C. for up to 36 months or 25° C. for up to 2 months. Glycation levels were monitored by cation exchange chromatography (CEX-UPLC) at 5° C. (FIG. 2A) or 25° C. (FIG. 2B), and HMW levels were monitored by size exclusion chromatography (SE-UPLC) at 5° C. (FIG. 2C) or 25° C. (FIG. 2D).

FIGS. 3A and 3B illustrate the effect of pH on the level of glycated and HMW species for mAb1 formulated in acetate buffer. The formulations included 50 mg/ml mAb1 in 10 mM acetate, and 5% w/v sucrose with different pH values, and were incubated at 40° C. for 28 days. Glycation levels were monitored by CEX-UPLC (FIG. 3A), and HMW levels were monitored by SE-UPLC (FIG. 3B).

FIG. 4 illustrates the effect of mAb1 concentration, sucrose concentration, and arginine concentration on the viscosity of mAb1 formulations. The plots represent statisitcal modeling of the experimental data.

FIG. 5 illustrates the effect of mAb1 concentration, sucrose concentration, and arginine concentration on the osmolality of mAb1 formulations. The plots represent statisitcal modeling of the experimental data.

FIG. 6 illustrates the effect of mAb1 concentration, sucrose concentration, and arginine concentration on the stability of mAb1 formulations. The plots represent statisitcal modeling of the experimental data.

FIGS. 7A and 7B illustrate the stability of two mAb1 formulations for subcutaneous administration. Both formulations demonstrate comparable stability when incubated under the tested conditions: 3 months at 40° C./75% relative humidity (RH); 6 months at 25° C./60% RH; and 6 months at 2-8° C. As shown in the figures, F1 contains 150 mg/ml mAb1, 30 mM acetate at pH 5.0, 8% w/v sucrose, and 0.05% w/v polysorbate 80, and F2 contains 150 mg/ml mAb1, 30 mM acetate at pH 5.0, 7% w/v sucrose, 50 mM arginine, and 0.05% w/v polysorbate 80.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, exemplary methods and materials are now described. All patents, applications and non-patent publications mentioned in this specification are incorporated herein by reference in their entireties.

### Pharmaceutical Formulations

As used herein, the expression “pharmaceutical formulation” means a combination of at least one active ingredient (e.g., a bispecific anti-MUC16×anti-CD3 antibody, which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present invention provides pharmaceutical formulations comprising at least one therapeutic polypeptide. According to certain embodiments of the present invention, the therapeutic polypeptide is a bispecific antibody that binds specifically to human MUC16 and human CD3 or an antigen-binding fragment thereof. More specifically, the present invention includes, inter alia, pharmaceutical formulations that comprise: (i) a human bispecific antibody that specifically binds to human MUC16 and human CD3; (ii) a buffer comprising acetate; (iii) an organic co-solvent comprising polysorbate; and (iv) a stabilizer comprising a sugar. Additional components may be included in the formulations of the present invention if such components do not significantly interfere with the stability of the formulation. Specific exemplary components and formulations included within the present invention are described in detail below.

The pharmaceutical formulations of the present invention may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

Bispecific Antibodies that Specifically Bind Human MUC16 and Human CD3

The pharmaceutical formulations of the present invention may comprise a human bispecific antibody, or an antigen-binding fragment thereof, that binds specifically to human MUC16 and human CD3.

The term “antibody,” as used herein, which includes a “bispecific antibody,” is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In certain embodiments of the invention, the anti-MUC16×anti-CD3 bispecific antibodies of the invention are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences. In various embodiments, the anti-MUC16×anti-CD3 bispecific antibody is a human IgG antibody. In various embodiments, the anti-MUC16×anti-CD3 bispecific antibody is a human antibody of isotype IgG1, IgG2, IgG3 or IgG4, or mixed isotype. In some embodiments, the anti-MUC16×anti-CD3 bispecific antibody is a human IgG1 antibody (i.e., the antibody comprises a human IgG1 heavy chain constant region attached, respectively, to the HCVR of each of the first antigen-binding domain and the second antigen-binding domain). In some embodiments, the anti-MUC16×anti-CD3 bispecific antibody is a human IgG4 antibody (i.e., the antibody comprises a human IgG4 heavy chain constant region attached, respectively, to the HCVR of each of the first antigen-binding domain and the second antigen-binding domain. In any of the embodiments discussed above or herein, the anti-MUC16×anti-CD3 bispecific antibody may comprise a human kappa light chain. In any of the embodiments discussed above or herein, the anti-MUC16×anti-CD3 bispecific antibody may comprise a human lambda light chain.

In any embodiments, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-MUC16 binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-MUC16 binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

In any embodiments, the bispecific antibody may include a chimeric hinge. The term “chimeric hinge” is intended to include a chimeric protein comprising a first amino acid sequence derived from the hinge region of one Ig molecule and a second amino acid sequence derived from the hinge region of a different class or subclass of Ig molecule. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “antigen-binding portion” or “antigen-binding fragment” of an antibody (or simply “antibody portion” or “antibody fragment”), as used herein, refer to one or more fragments of an antibody that retain the ability to specifically bind to human MUC16 or human CD3.

An “isolated antibody,” as used herein, is intended to refer to an antibody that is substantially free of other antibodies having different antigenic specificities (e.g., an isolated bispecific antibody that specifically binds human MUC16 and human CD3 is substantially free of antibodies that specifically bind antigens other than human MUC16 and human CD3).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds human MUC16 and human CD3 may, however, have cross-reactivity to other antigens, such as MUC16 or CD3 molecules from other species (orthologs). In the context of the present invention, multispecific (e.g., bispecific) antibodies that bind to human MUC16 and human CD3 as well as one or more additional antigens are deemed to “specifically bind” human MUC16 and human CD3. Moreover, an isolated antibody may be substantially free of other cellular material and/or chemicals.

Exemplary anti-MUC16×anti-CD3 bispecific antibodies that may be included in the pharmaceutical formulations of the present invention are set forth in WO 2018/067331, the disclosure of which is incorporated by reference in its entirety.

According to certain embodiments of the present invention, the anti-MUC16×anti-CD3 bispecific antibody, or antigen-binding fragment thereof, comprises a first antigen-binding domain that specifically binds human MUC16 and a second antigen-binding domain that specifically binds human CD3, in which the first antigen-binding domain comprises heavy chain complementarity determining regions (CDRs) A1-HCDR1, A1-HCDR2, and A1-HCDR3, respectively, comprising the amino acid sequences of SEQ ID NOs: 7, 8, and 9, and the second antigen-binding domain comprises heavy chain CDRs A2-HCDR1, A2-HCDR2, and A2-HCDR3, respectively, comprising the amino acid sequences of SEQ ID NOs: 10, 11, and 12. According to certain embodiments of the present invention, the anti-MUC16×anti-CD3 bispecific antibody, or antigen-binding fragment thereof, comprises common (to both the first and second antigen-binding domains) light chain complementarity determining regions LCDR1-LCDR2-LCDR3, respectively, comprising the amino acid sequences of SEQ ID NOs: 13, 14, and 15.

In certain embodiments, the anti-MUC16×anti-CD3 bispecific antibody, or antigen-binding fragment thereof, comprises a first antigen-binding domain that specifically binds human MUC16 and a second antigen-binding domain that specifically binds human CD3, in which the first antigen-binding domain comprises a heavy chain variable region (HCVR) comprising the amino acid sequence of SEQ ID NO: 4, and the second antigen-binding domain comprises a HCVR comprising the amino acid sequence of SEQ ID NO: 5. In certain embodiments, the anti-MUC16×anti-CD3 bispecific antibody, or antigen-binding fragment thereof, comprises a common light chain variable region (LCVR) comprising the amino acid sequence of SEQ ID NO: 6. In certain embodiments, the anti-MUC16×anti-CD3 bispecific antibody, or antigen-binding fragment thereof, comprises a first antigen-binding domain comprising a HCVR/LCVR amino acid sequence pair comprising the amino acid sequences of SEQ ID NOs: 4/6, and a second antigen-binding domain comprising a HCVR/LCVR amino acid sequence pair comprising the amino acid sequences of SEQ ID NOs: 5/6. In some embodiments, the anti-MUC16×anti-CD3 bispecific antibody comprises the HCVR/LCVR sequence pairs noted above, and a human IgG1 heavy chain constant region. In some embodiments, the anti-MUC16×anti-CD3 bispecific antibody comprises the HCVR/LCVR sequence pairs noted above, and a human IgG4 heavy chain constant region. In some embodiments, the anti-MUC16×anti-CD3 bispecific antibody comprises the HCVR/LCVR sequence pairs noted above, and a human IgG heavy chain constant region. In some embodiments, the anti-MUC16×anti-CD3 bispecific antibody comprises the HCVR/LCVR sequence pairs noted above, and a human IgG1 or IgG4 heavy chain constant region. In some embodiments, the anti-MUC16×anti-CD3 bispecific antibody comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3. An anti-MUC16×anti-CD3 bispecific antibody with a first antigen-binding domain that specifically binds human MUC16 and comprises a HCVR comprising the amino acid sequence of SEQ ID NO: 4 and a LCVR comprising the amino acid sequence of SEQ ID NO: 6, and a second antigen-binding domain that specifically binds human CD3 and comprises a HCVR comprising the amino acid sequence of SEQ ID NO: 5 and a LCVR comprising the amino acid sequence of SEQ ID NO: 6 is referred to herein as mAb1. This antibody has a first heavy chain (including the HCVR that specifically binds human MUC16) comprising the amino acid sequence of SEQ ID NO: 1, a second heavy chain (including the HCVR that specifically binds human CD3) comprising the amino acid sequence of SEQ ID NO: 2, and a common light chain comprising the amino acid sequence of SEQ ID NO: 3. In some cases, the mature form of the antibody may not include the C-terminal lysine residues of SEQ ID NOs: 1 and 2. Thus, in some cases the anti-MUC16 binding arm of mAb1 comprises a heavy chain comprising residues 1-442 of SEQ ID NO: 1, and the anti-CD3 binding arm of mAb1 comprises a heavy chain comprising residues 1-449 of SEQ ID NO: 2.

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments, the pharmaceutical formulations may contain about 0.1 mg/mL to about 500 mg/mL of antibody; about 0.5 mg/mL to about 400 mg/mL of antibody; about 1 mg/mL to about 200 mg/mL of antibody; about 2 mg/mL to about 100 mg/mL; about 1 mg/mL to about 5 mg/mL of antibody; about 10 mg/mL to about 30 mg/mL of antibody; about 75 mg/mL to about 125 mg/mL; about 5 mg/mL to about 50 mg/mL; about 4 mg/ml to about 60 mg/ml; or about 2 mg/mL to about 55 mg/mL of antibody. For example, the formulations of the present invention may by liquid formulations that comprise about 0.5 mg/mL; about 1 mg/mL; about 2 mg/mL; about 3 mg/mL; about 4 mg/mL; about 5 mg/mL; about 6 mg/mL; about 7 mg/mL, about 8 mg/mL; about 9 mg/mL; about 10 mg/mL; about 11 mg/mL; about 12 mg/mL; about 13 mg/mL; about 14 mg/mL; about 15 mg/mL; about 16 mg/mL; about 17 mg/mL; about 18 mg/mL; about 19 mg/mL; about 20 mg/mL; about 21 mg/mL; about 22 mg/mL; about 23 mg/mL; about 24 mg/mL; about 25 mg/mL; about 26 mg/mL; about 27 mg/mL; about 28 mg/mL; about 29 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 96 mg/mL; about 97 mg/mL; about 98 mg/mL; about 99 mg/mL; about 100 mg/mL; about 101 mg/mL; about 102 mg/mL; about 103 mg/mL; about 104 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to human MUC16 and human CD3. In certain embodiments, the pharmaceutical formulations are liquid formulations that may contain 1±0.1 mg/mL to 200±20 mg/mL of antibody; 2±0.2 mg/mL to 10±1 mg/mL of antibody; 1±0.5 mg/mL to 30±5 mg/mL of antibody; 40±4 mg/mL to 60±6 mg/mL of antibody; 1±0.1 mg/mL to 3±0.3 mg/mL of antibody; 3±0.5 mg/mL to 7±0.5 mg/mL of antibody; 45±1 mg/mL to 55±1 mg/mL of antibody; 140±5 mg/ml to 160±5 mg/ml of antibody; or 175±5 mg/mL to 185±5 mg/mL of antibody. In some embodiments, the pharmaceutical formulations contain 5±0.5 mg/mL of antibody. In some embodiments, the pharmaceutical formulations contain 50±5 mg/mL of antibody. In some embodiments, the pharmaceutical formulations contain 150±15 mg/ml of antibody. In some embodiments, the pharmaceutical formulations contain 2±0.2 mg/ml of antibody. In some embodiments, the pharmaceutical formulations contain 20±2 mg/ml of antibody. In some embodiments, the pharmaceutical formulations contain 180±10 mg/ml of antibody.

### Bioequivalents

The present invention encompasses antibodies having amino acid sequences that vary from those of the exemplary molecules disclosed herein but that retain the ability to bind human MUC16 and human CD3. Such variant molecules may comprise one or more additions, deletions, or substitutions of amino acids when compared to parent sequence, but exhibit biological activity that is essentially equivalent to that of the antibodies discussed herein.

The present invention includes antigen-binding molecules that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods.

Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

### Formulation Excipients and pH

The pharmaceutical formulations of the present invention comprise one or more excipients. The term “excipient,” as used herein, means any non-therapeutic agent added to the formulation to provide a desired consistency, viscosity or stabilizing effect.

In certain embodiments, the pharmaceutical formulations of the present invention comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some embodiments, the sugar is sucrose. In some cases, the sugar (e.g., sucrose) acts as a thermal stabilizer for the anti-MUC16×anti-CD3 bispecific antibody.

The amount of sugar (e.g., sucrose) contained within the pharmaceutical formulations of the present invention will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 5% to about 15% sugar; about 7.5% to about 12.5% sugar; or about 9% to about 11% sugar. For example, the pharmaceutical formulations of the present invention may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 10.5%; about 11.0%; about 11.5%; about 12.0%; about 12.5%; about 13.0%; about 13.5%; about 14.0%; about 14.5%; about 15%; or about 20% sugar (e.g., sucrose). In some embodiments, the formulations contain about 10% sugar (e.g., sucrose). In some embodiments, the formulations contain about 5% sugar (e.g., sucrose). Each of the percentages noted above corresponds to a percent weight/volume (w/v). In some cases, the formulations contain from 5%±1% to 20%±4% w/v sucrose. In some cases, the formulations contain from 5% to 10% w/v sucrose. In some cases, the formulations contain from 8%±0.5% to 12%±0.5% w/v sucrose. In some cases, the formulations contain 10%±1% w/v sucrose.

The pharmaceutical formulations of the present invention may also comprise one or more organic co-solvents (or interfacial stabilizer) in a type and in an amount that stabilizes the anti-MUC16×anti-CD3 bispecific antibody under conditions of rough handling or agitation, such as, e.g., orbital shaking. In some embodiments, the organic co-solvent is a surfactant. As used herein, the term “surfactant” means a substance which reduces the surface tension of a fluid in which it is dissolved and/or reduces the interfacial tension between oil and water. Surfactants can be ionic or non-ionic. Exemplary non-ionic surfactants that can be included in the formulations of the present invention include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations of the present invention include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 188 (also known as Pluronic F68), poloxamer 407; polyethylene-polypropylene glycol; or polyethylene glycol (PEG). Polysorbate 20 is also known as TWEEN 20, sorbitan monolaurate and polyoxyethylenesorbitan monolaurate. In some embodiments, the surfactant is polysorbate 20.

The amount of surfactant contained within the pharmaceutical formulations of the present invention may vary depending on the specific properties desired of the formulations, as well as the particular circumstances and purposes for which the formulations are intended to be used. In certain embodiments, the formulations may contain about 0.01% to about 1% surfactant; about 0.01% to about 0.5% surfactant; about 0.1% to about 0.3%; about 0.15% to about 0.25% surfactant; or about 0.19% to about 0.21% surfactant. For example, the formulations of the present invention may comprise about 0.01%; about 0.02%; about 0.03%; about 0.04%; about 0.05%; about 0.06%; about 0.07%; about 0.08%; about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% surfactant (e.g., polysorbate 20). In some embodiments, the formulations contain about 0.2% surfactant (e.g., polysorbate 20). In some embodiments, the formulations contain about 0.05% surfactant (e.g., polysorbate 20). Each of the percentages noted above corresponds to a percent weight/volume (w/v). In some cases, the formulations contain from 0.01%±0.005% to 0.5%±0.25% w/v polysorbate 20. In some cases, the formulations contain 0.2%±0.05% w/v polysorbate 20. In some cases, the formulations contain 0.2%±0.01% w/v polysorbate 20.

The pharmaceutical formulations of the present invention may also comprise a buffer or buffer system, which serves to maintain a stable pH and to help stabilize the anti-MUC16×anti-CD3 bispecific antibody. In some embodiments, the buffer or buffer system comprises at least one buffer that has a buffering range that overlaps fully or in part the range of pH 4.5 to 5.5. In certain embodiments, the buffer comprises an acetate buffer (e.g., sodium acetate). In certain embodiments, the buffer (e.g., acetate) is present at a concentration of from about 1 mM to about 50 mM, about 20 mM to about 40 mM, about 25 mM to about 35 mM; about 28 mM to about 32 mM; or about 29 mM to about 31 mM. In some embodiments, the buffer (e.g., acetate) is present at a concentration of about 20 mM; about 21 mM; about 22 mM; about 23 mM; about 24 mM; about 25 mM; about 26 mM; about 27 mM; about 28 mM; about 29 mM; about 30 mM; about 31 mM; about 32 mM; about 33 mM; about 34 mM; about 35 mM; about 36 mM; about 37 mM; about 38 mM; about 39 mM; or about 40 mM. In some case, the buffer is a histidine buffer present at a concentration of about 1 mM; about 2 mM; about 3 mM; about 4 mM; about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; or about 20 mM. In some cases, the formulations contain a histidine buffer at a concentration of from 5 mM±1 mM to 15 mM±3 mM. In some cases, the formulations contain a histidine buffer at a concentration of 10 mM±1 mM. In some embodiments, the formulations contain an acetate buffer (e.g., at any of the concentrations discussed above or herein). In some embodiments, the formulations contain a phosphate buffer (e.g., at any of the concentrations discussed above or herein).

In some embodiments, the pharmaceutical formulations of the present invention may also comprise arginine. In some cases, arginine is present at a concentration of from 1 to 100 mM. In some embodiments, arginine is present at a concentration of from 25 to 75 mM. In some cases, the arginine is present at a concentration of 50 mM±5 mM. in one embodiment, the pharmaceutical formulation comprises 30 mM±3 mM acetate at pH 5.0±0.1, 7%±0.7% w/v sucrose, 0.05%±0.01% w/v polysorbate (e.g., polysorbate 20), and 50 mM±5 mM arginine. In some cases, the antibody is present at a concentration of from 1 mg/ml to 200 mg/ml, or 150 mg/ml±10 mg/ml.

During the antibody purification process it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect (Bolton et al., 2011, Biotechnol. Prog. 27(1):140-152). The buildup of positive charge on the product side of the membrane during protein concentration is counterbalanced electrically by the preferential movement of positive ions to the opposite side of the membrane. The potential consequence of this phenomenon is that the final concentrations of certain components (e.g., acetate) may be lower than the intended target concentrations of these components due to the electrostatic repulsion of positively charged diafiltration buffer excipients to the positively charged antibody protein during the UF/DF step. Thus, the present invention includes formulations in which the concentration of, e.g., acetate vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present invention includes formulations in which the concentration of, e.g., acetate may vary from the recited amounts or ranges herein due to the volume exclusion effect.

During the manufacture of the formulations of the present invention, variations in the composition of the formulation may occur. These variations may include the concentration of the active ingredient, the concentration of the excipients, and/or the pH of the formulation. The present invention includes formulations comprising anti-MUC16×anti-CD3 bispecific antibodies which are stable and retain potency with up to at least 10% variation in the excipient concentration. For example, included herein are anti-MUC16×anti-CD3 bispecific antibody formulations, wherein stability and potency of the formulations is unaffected by ±10%, or ±20% variation in the concentration of antibody, sucrose, acetate buffer and/or polysorbate.

### Stability of the Pharmaceutical Formulations

The pharmaceutical formulations of the present invention exhibit high levels of stability. The term “stable,” as used herein in reference to the pharmaceutical formulations, means that the antibodies within the pharmaceutical formulations retain an acceptable degree of structure and/or function and/or biological activity after storage for a defined amount of time. A formulation may be stable even though the antibody contained therein does not maintain 100% of its structure and/or function and/or biological activity after storage for a defined amount of time. Under certain circumstances, maintenance of about 90%, about 95%, about 96%, about 97%, about 98% or about 99% of an antibody's structure and/or function and/or biological activity after storage for a defined amount of time may be regarded as “stable.”

Stability can be measured by, inter alia, determining the percentage of native antibody remaining in the formulation after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C., or about 45° C. For example, a pharmaceutical formulation may be deemed stable if after 3 months of storage at 5° C., greater than about 90%, 95%, 96% or 97% of native antibody is detected by SE-HPLC. A pharmaceutical formulation may also be deemed stable if after 6 months of storage at 5° C., greater than about 90%, 95%, 96% or 97% of native antibody is detected by SE-HPLC. A pharmaceutical formulation may also be deemed stable if after 9 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 12 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 24 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 36 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 3 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 6 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 9 months of storage at 25° C. (and optionally 60% relative humidity), greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 3 months of storage at 37° C., greater than about 90%, 95%, 96% or 97% of native antibody is detected by SE-HPLC. A pharmaceutical formulation may also be deemed stable if after 1 month of storage at 45° C. (and optionally 75% relative humidity), greater than about 90%, 91%, 92%, 93%, 94%, 95%, 96%, 97% or 98% of native antibody is detected by SE-HPLC.

Other methods may be used to assess the stability of the formulations of the present invention such as, e.g., differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at t=0.

Measuring the binding affinity of the antibody to its target may also be used to assess stability. For example, a formulation of the present invention may be regarded as stable if, after storage at e.g., −80° C., −30° C., −20° C., 5° C., 25° C., 37° C., 45° C., etc. for a defined amount of time (e.g., 14 days to 9 months), the anti-MUC16×anti-CD3 bispecific antibody contained within the formulation binds to human MUC16 and human CD3 with an affinity that is at least 80%, 85%, 90%, 95%, or more of the binding affinity of the antibody prior to said storage. Binding affinity may be determined by any method, such as e.g., ELISA or plasmon resonance. Biological activity may be determined by a MUC16 or CD3 activity assay, such as by contacting a cell that expresses MUC16 or CD3 with the formulation comprising the anti-MUC16×anti-CD3 bispecific antibody. The binding of the antibody to such a cell may be measured directly, such as via FACS analysis.

Stability can be measured, inter alia, by determining the percentage of antibody that forms an aggregate (high molecular weight (HMW) species) within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC] or size exclusion ultra-performance liquid chromatography [SE-UPLC]). An “acceptable degree of stability”, as that phrase is used herein, means that at most 6% of the antibody is in an aggregated form detected in the formulation after storage for a defined amount of time at a given temperature (up to 25° C.). In certain embodiments an acceptable degree of stability means that at most about 6%, 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C. or about 45° C. For example, a pharmaceutical formulation may be deemed stable if after twelve months of storage at 5° C., less than about 3%, 2.75%, 2.5%, 2.25%, 2%, 1.75%, 1.5%, 1.25%, 1%, 0.75%, 0.5%, 0.25%, or 0.1% of the antibody is detected in an aggregated form. In some cases, a pharmaceutical formulation may be deemed stable if after six months of storage at 5° C., less than about 5%, 4.75%, 4.5%, 4.25%, 4%, 3.75%, 3.5%, 3.25%, 2% or 1% of the antibody is detected in an aggregated form. A pharmaceutical formulation may also be deemed stable if after six months of storage at 25° C. and 60% relative humidity, less than about 6%, 5.75%, 5.5%, 5.25%, 5%, 4.5%, 4%, 3.5%, 3%, 2.5%, 2%, 1.75%, 1.5%, 1.25%, 1%, 0.75%, 0.5%, 0.25%, or 0.1% of the antibody is detected in an aggregated form. A pharmaceutical formulation may also be deemed stable if after three months of storage at 37° C., less than about 6%, 5.75%, 5.5%, 5.25%, 5%, 4.5%, 4%, 3.5%, 3%, 2.5%, 2%, 1.75%, 1.5%, 1.25%, 1%, 0.75%, 0.5%, 0.25%, or 0.1% of the antibody is detected in an aggregated form. A pharmaceutical formulation may also be deemed stable if after twelve months of storage at −30° C., or −80° C. less than about 3%, 2.75%, 2.5%, 2.25%, 2%, 1.9%, 1.8%, 1.7%, 1.6%, 1.5%, 1%, 0.5%, or 0.1% of the antibody is detected in an aggregated form.

Stability can be measured, inter alia, by determining the percentage of antibody that remains in the form of a glycated species. The percentage of “glycated species” of antibody can be determined by ion exchange chromatography (e.g., cation exchange high performance liquid chromatography [CEX-HPLC] or cation exchange ultra-performance liquid chromatography [CEX-UPLC]) and/or by LC-MS. An “acceptable degree of stability”, as that phrase is used herein, means that the percentage change in the percentage of antibody in the form of a “glycated species” does not exceed a specified amount after storage for a defined amount of time at a defined temperature. In certain embodiments an acceptable degree of stability means that the percentage change in “glycated species” is no more than 25%, no more than 20%, no more than 15%, no more than 10%, no more than 9%, no more than 8%, no more than 7%, no more than 6%, no more than 5%, no more than 4%, no more than 3%, no more than 2.5%, no more than 2%, no more than 1.5%, or no more than 1% after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., or about 45° C. For example, a pharmaceutical formulation may be deemed stable if after twelve months of storage at −80° C. or −30° C., the percentage change in “glycated species” is no more than 5%, no more than 4%, no more than 3%, no more than 2%, or no more than 1.5%. In another example, a pharmaceutical formulation may be deemed stable if after six months of storage at 5° C., the percentage change in “glycated species” is no more than 5%, no more than 4%, no more than 3%, no more than 2%, or no more than 1.5%. In another example, a pharmaceutical formulation may be deemed stable if after twelve months of storage at 5° C., the percentage change in “glycated species” is no more than 5%, no more than 4%, or no more than 3%. In each case, the measurement may be performed using cation exchange ultra performance liquid chromatography (CEX-UPLC) and/or by LC-MS.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 12 months means that the measurement of stability was taken at or about 12 months after the start of the study. Additional methods for assessing the stability of an antibody in formulation are demonstrated in the Examples presented below.

As illustrated in the Examples below, the present invention is based, in part, on the discovery that the combination of claimed excipients with a bispecific anti-MUC16×anti-CD3 antibody produces a formulation that is stable.

### Exemplary Formulations

According to one aspect of the present invention, the pharmaceutical formulation comprises: (i) a human anti-MUC16×anti-CD3 bispecific antibody that specifically binds to human MUC16 and human CD3; (ii) a buffer comprising acetate (e.g., sodium acetate); (iii) an organic co-solvent comprising polysorbate; and (iv) a stabilizer comprising a sugar. According to another aspect, the pharmaceutical formulation comprises: (i) a human anti-MUC16×anti-CD3 bispecific antibody that specifically binds to human MUC16 and human CD3; (ii) a buffer comprising acetate; and (iii) a stabilizer comprising a sugar. According to another aspect, the pharmaceutical formulation comprises: (i) a human anti-MUC16×anti-CD3 bispecific antibody that specifically binds to human MUC16 and human CD3; (ii) a buffer comprising histidine; (iii) an organic co-solvent comprising polysorbate; and (iv) a stabilizer comprising a sugar.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml; (ii) acetate at a concentration of from about 25 mM to about 35 mM; (iii) polysorbate 20 at a concentration of from about 0.1% w/v to about 0.3% w/v; and (iv) sucrose at a concentration of from about 5% w/v to about 15% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 25 mM to about 35 mM; (iii) polysorbate 20 at a concentration of from about 0.1% w/v to about 0.3% w/v; and (iv) sucrose at a concentration of from about 5% w/v to about 15% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 25 mM to about 35 mM; (iii) polysorbate 20 at a concentration of from about 0.1% w/v to about 0.3% w/v; and (iv) sucrose at a concentration of from about 5% w/v to about 15% w/v, wherein the formulation has a pH of 5.0±0.3.

In some cases, the stable liquid pharmaceutical formulation comprises (i) a human bispecific antibody that specifically binds to human MUC16 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 1 mg/ml to about 200 mg/ml; (ii) acetate at a concentration of from about 25 mM to about 35 mM; (iii) polysorbate 20 at a concentration of from about 0.1% w/v to about 0.3% w/v; and (iv) sucrose at a concentration of from about 5% w/v to about 15% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 1 mg/ml to about 200 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml; (ii) acetate at a concentration of from about 30 mM; (iii) polysorbate 20 at a concentration of from about 0.2% w/v; and (iv) sucrose at a concentration of from about 10% w/v, wherein the formulation has a pH of 5.0±0.1.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM; (iii) polysorbate 20 at a concentration of from about 0.2% w/v; and (iv) sucrose at a concentration of from about 10% w/v, wherein the formulation has a pH of 5.0±0.1.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM; (iii) polysorbate 20 at a concentration of from about 0.2% w/v; and (iv) sucrose at a concentration of from about 10% w/v, wherein the formulation has a pH of 5.0±0.1.

In some cases, the stable liquid pharmaceutical formulation comprises (i) a human bispecific antibody that specifically binds to human MUC16 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 1 mg/ml to about 200 mg/ml; (ii) histidine at a concentration of 10 mM; (ii) acetate at a concentration of from about 30 mM; (iii) polysorbate 20 at a concentration of from about 0.2% w/v; and (iv) sucrose at a concentration of from about 10% w/v, wherein the formulation has a pH of 5.0±0.1.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 3 mg/ml±1 mg/ml to 7 mg/ml±1 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 5 mg/ml±0.5 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 40 mg/ml±2 mg/ml to 60 mg/ml±2 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 50 mg/ml±5 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 5 mg/ml±0.5 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 50 mg/ml±5 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 5 mg/ml±0.5 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 50 mg/ml±0.5 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from 5 mg/ml±0.5 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 50 mg/ml±5 mg/ml; (ii) acetate at a concentration of from about 30 mM±1 mM; (iii) polysorbate 20 at a concentration of from about 0.2% w/v±0.02% w/v; and (iv) sucrose at a concentration of from about 10% w/v±1% w/v, wherein the formulation has a pH of 5.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 1 mg/ml to about 3 mg/ml; (ii) histidine at a concentration of from about 10 mM±1 mM; (iii) polysorbate 20 at a concentration of from about 0.05% w/v±0.01% w/v; and (iv) sucrose at a concentration of from about 10% w/v±1% w/v, wherein the formulation has a pH of 6.0±0.3.

In some cases, the stable liquid pharmaceutical formulation comprises (i) a human bispecific antibody that specifically binds to human MUC16 and human CD3 and comprises a first antigen-binding domain that specifically binds human MUC16 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 150 mg/ml to about 200 mg/ml; (ii) acetate at a concentration of from about 30 mM±1 mM; and (iii) sucrose at a concentration of from about 5% w/v±0.5% w/v, wherein the formulation has a pH of 5.0±0.3.

In any of these exemplary formulations, “stable” may be defined as: (a) the formulation contains no more than 2.5% high molecular weight (HMVV) species after 12 month of storage at 5° C., as determined by SE-UPLC; (b) the formulation contains no more than 3.5% high molecular weight (HMVV) species after 6 month of storage at 25° C. and 60% relative humidity, as determined by SE-UPLC; (c) the formulation contains no more than 1.5% high molecular weight (HMVV) species after 12 month of storage at −30° C., as determined by SE-UPLC; (d) the formulation contains no more than 1.5% high molecular weight (HMVV) species after 12 month of storage at −80° C., as determined by SE-UPLC; (e) at least 95% of the antibody has native conformation after 12 months of storage at 5° C. as determined by SE-UPLC; (f) at least 95% of the antibody has native conformation after 6 months of storage at 25° C. and 60% relative humidity as determined by SE-UPLC; (g) at least 95% of the antibody has native conformation after 3 months of storage at 37° C. as determined by SE-UPLC; (h) the formulation contains no more than 1% high molecular weight (HMW) species after 12 months of storage at 5° C. as determined by SE-UPLC; (i) the formulation contains no more than 1% HMW species after 6 months of storage at 25° C. and 60% relative humidity as determined by SE-UPLC; (j) the formulation contains no more than 1% HMW species after 3 months of storage at 37° C.; as determined by SE-UPLC; (k) the formulation contains no more than 2% high molecular weight (HMVV) species after 12 months of storage at −30° C. or −80 C as determined by SE-UPLC; (I) the formulation contains no more than 4% HMW species after 6 months of storage at 5° C. as determined by SE-UPLC; or (m) the formulation contains no more than 6% HMW species after 6 months of storage at 25° C. and 60% relative humidity as determined by SE-UPLC.

In any of these exemplary formulations, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-MUC16 binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-MUC16 binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

Additional non-limiting examples of pharmaceutical formulations encompassed by the present invention are set forth elsewhere herein, including the working Examples presented below.

### Containers and Methods of Administration

The pharmaceutical formulations of the present invention may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present invention including, e.g., clear and opaque (e.g., amber) glass or plastic vials. Likewise, any type of syringe can be used to contain and/or administer the pharmaceutical formulations of the present invention. In some embodiments, the pharmaceutical formulation is contained in a prefilled syringe. In some embodiments, the pharmaceutical formulation is contained in a prefilled staked needle syringe.

The pharmaceutical formulations of the present invention may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present invention, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present invention, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present invention are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present invention are commercially available under the tradename “FluroTec®,” available from West Pharmaceutical Services, Inc. (Lionville, Pa.). FluroTec® is an example of a flurocarbon coating used to minimize or prevent drug product from adhering to the rubber surfaces. According to certain embodiments of the present invention, the pharmaceutical formulations may be contained within a low tungsten syringe that comprises a fluorocarbon-coated plunger. In some embodiments, the container is a syringe, such as an Ompi EZ-Fill™ syringe or a BD Neopak™ syringe. In some cases, the syringe is a 1 mL long glass syringe with a 1 mL iWest piston, a 27 G thin wall needle and an FM30 needle shield or a BD260 needle shield. In some cases, the syringe is a 2.25 mL glass syringe with a West NovaPure™ 1-3 mL piston, a 27 G thin wall needle and an FM30 needle shield or a BD260 needle shield. In various embodiments, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 mL syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present invention is also contemplated herein. In some cases, the LVD or bolus injector may be configured to inject a medicament into a patient. For example, an LVD or bolus injector may be configured to deliver a “large” volume of medicament (typically about 2 ml to about 10 ml).

In certain embodiments, the pharmaceutical formulation is administered via an IV drip, such that the formulation is diluted in an IV bag containing a physiologically acceptable solution. In one embodiment, the pharmaceutical composition is a compounded sterile preparation in an intravenous infusion bag, such that a single dose of drug product is diluted into 100 mL, 250 mL (or other like amount suitable for intravenous drip delivery) of a physiological buffer (e.g., 0.9% saline).

The pharmaceutical formulations of the present invention can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-MUC16×anti-CD3 bispecific antibody) in the formulations of the present invention may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient (e.g., an anti-MUC16×anti-CD3 bispecific antibody) intended for a single use. In various embodiments, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 40 mg to about 60 mg, from about 125 mg to about 175 mg, from about 160 mg to about 200 mg, from about 1 mg to about 250 mg, from about 1 mg to about 100 mg, from about 1 mg to about 50 mg, from about 1 mg to about 25 mg, from about 1 mg to about 20 mg, from about 5 mg to about 15 mg, or ranges or intervals thereof. Ranges intermediate to the above recited amounts, for example, from about 2 mg to about 100 mg or 2 mg to 20 mg, are also intended to be part of this invention. For example, ranges of values using a combination of any of the above recited values (or values contained within the above recited ranges) as upper and/or lower limits are intended to be included. In some embodiments, the unit dosage form contains 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 mg of the antibody. In some embodiments, the unit dosage form contains 5 mg of the antibody. In some embodiments, the unit dosage form contains 12.5 mg of the antibody. In a particular embodiment, the formulation often is supplied as a liquid in unit dosage form. In some embodiments, the unit dosage form contains from 3 to 7 mg, or from 8 to 12 mg, from 10 to 15 mg, from 35 to 45 mg, from 45 to 55 mg, from 140 to 160 mg, or from 170 to 190 mg. In some embodiments, a unit dosage form according to the present invention is suitable for subcutaneous administration to a patient (e.g., a unit dosage form containing the antibody at a concentration of about 100 mg/ml or about 200 mg/ml, or 150 mg/ml±5 mg/ml).

The present invention also includes methods of preparing a unit dosage form. In an exemplary embodiment, a method for preparing a pharmaceutical unit dosage form includes combining the formulation of any of foregoing embodiments in a suitable container (e.g., those containers discussed herein).

### Therapeutic Uses of the Pharmaceutical Formulations

The pharmaceutical formulations of the present invention are useful, inter alia, for the treatment, prevention and/or amelioration of any disease or disorder associated with a cell expressing human MUC16. Exemplary, non-limiting diseases and disorders that can be treated by the administration of the pharmaceutical formulations of the present invention include ovarian cancer, breast cancer, pancreatic cancer, and non-small-cell lung cancer.

The therapeutic methods of the present invention comprise administering to a subject any formulation comprising an anti-MUC16×anti-CD3 bispecific antibody as disclosed herein. The subject to which the pharmaceutical formulation is administered can be, e.g., any human or non-human animal that is in need of such treatment. For example, the subject can be an individual that is diagnosed with, or who is deemed to be at risk of being afflicted by any of the aforementioned diseases or disorders. The present invention further includes the use of any of the pharmaceutical formulations disclosed herein in the manufacture of a medicament for the treatment of any disease or disorder associated with a cell expressing human MUC16, including any of the above mentioned exemplary diseases, disorders and conditions.

In some embodiments, the present invention provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Summary of Sequences

| SEQ ID NO: | Description |
|---|---|
| 1 | Anti-MUC16 Heavy Chain |
| 2 | Anti-CD3 Heavy Chain |
| 3 | Common Anti-MUC16 and Anti-CD3 Light Chain |
| 4 | Anti-MUC16 HCVR |
| 5 | Anti-CD3 HCVR |
| 6 | Common Anti-MUC16 and Anti-CD3 LCVR |
| 7 | Anti-MUC16 HCDR1 |
| 8 | Anti-MUC16 HCDR2 |
| 9 | Anti-MUC16 HCDR3 |
| 10 | Anti-CD3 HCDR1 |
| 11 | Anti-CD3 HCDR2 |
| 12 | Anti-CD3 HCDR3 |
| 13 | Common Anti-MUC16 and Anti-CD3 LCDR1 |
| 14 | Common Anti-MUC16 and Anti-CD3 LCDR2 |
| 15 | Common Anti-MUC16 and Anti-CD3 LCDR3 |
| 16 | IgG4 Heavy Chain Constant Region |
| 17 | IgG4 Heavy Chain Constant Region with H435R/Y436F |
| 18 | IgG1 Heavy Chain Constant Region |
| 19 | IgG1 Heavy Chain Constant Region with H435R/Y436F |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Development of Stable Liquid and Lyophilized Anti-MUC16×Anti-CD3 Bispecific Antibody Formulations

The physical stability of a formulation refers to properties such as color, appearance, pH, turbidity, particulates, and protein concentration. The chemical stability refers to the formation of high-molecular-weight (HMW) species, low-molecular-weight (LMW) species, charge variants, and other chemical modifications of the protein. The physical and chemical stabilities of mAb1 drug product were assessed using the following assays: Color and appearance by visual inspection; pH; Turbidity measured by increase in optical density (OD) at 405 nm; Subvisible particulate analysis by Micro-Flow Imaging™ (MFI); Protein concentration by reversed-phase ultra performance liquid chromatography (RP-UPLC); Purity of each individual drug product was assessed using size-exclusion ultra performance liquid chromatography (SE-UPLC), and reduced and non-reduced microchip capillary electrophoresis-sodium dodecyl sulfate (MCESDS); Charge variant analysis was determined using cation exchange UPLC (CEX-UPLC) (glycated mAb1 was detected by CEX-UPLC), and imaged capillary isoelectric focusing (iCIEF); and Potency by bioassay (the relative potency of each sample was determined by bioassay and is defined as: (IC₅₀ Reference Sample/IC₅₀ Sample)×100%; the measured potency of storage stability samples must be within 50-150% of the measured potency of the reference standard).

A lyophilized formulation of mAb1 was developed for intravenous (IV) or subcutaneous (SC) administration. The lyophilized mAb1 drug product can be reconstituted with sterile Water for Injection (WFI) to a concentration of 2 mg/mL mAb1 or 20 mg/ml mAb1 for IV infusion or SC injection. Formulation development activities involved assessment of buffers, pH, organic co-solvents, surfactants, and sucrose (as the thermal stabilizer) to identify excipients that enhance protein stability. Results generated from these studies were used to develop a stable lyophilized formulation that was suitable for reconstitution to liquid form.

### Buffer and pH Selection

The effect of buffer and pH on the thermal stability of mAb1 was initially examined in liquid formulations by incubating 2 mg/mL mAb1 at 45° C. for 28 days in a series of buffer systems at varying pH. The following pH and buffer systems were studied: acetate (pH 4.5 to 5.5), histidine (pH 5.5 to 6.5), and phosphate (pH 6.5 to 7.5). These analyses revealed that formation of HMW species and charge variants were the main degradation pathways. Based on results from SE-UPLC, the lowest rate of HMW species formation and the lowest rate of monomer loss were observed when mAb1 was formulated between pH 5.5 and 6.5 in histidine buffer or between pH 4.5 and 5.5 in acetate buffer, as shown in Table 2. CEX-UPLC analysis also indicated that the charge variant profile was most stable when mAb1 was formulated between pH 5.5 and 6.5 in histidine buffer or between pH 4.5 and 5.5 in acetate buffer, as shown in Table 2. Relative to the starting material, a decrease in the relative amount of total acidic species and a concomitant increase in the main charge variant peak was observed by CEX-UPLC analysis following incubation of mAb1 at 45° C. for 28 days.

A single peak that comprised approximately 50% of the total acidic charge variants observed in the CEX-UPLC chromatogram was isolated. Further analysis identified the primary component in this peak as mAb1 protein containing a glycation of heavy chain (HC) Lys98 within CDR3 of the MUC16 binding arm of the bispecific antibody (the glycation is discussed in detail in the following paragraph). During initial activities associated with the development of the mAb1 formulation, the stability of the main charge variant, the total acidic species, the total basic species, and the glycated form of the molecule were all monitored. Histidine buffer, pH 6.0, was chosen for the clinically enabling formulation since the rate of HMW species formation, rate of monomer loss, and rate of charge variant formation were minimal in this buffer and pH.

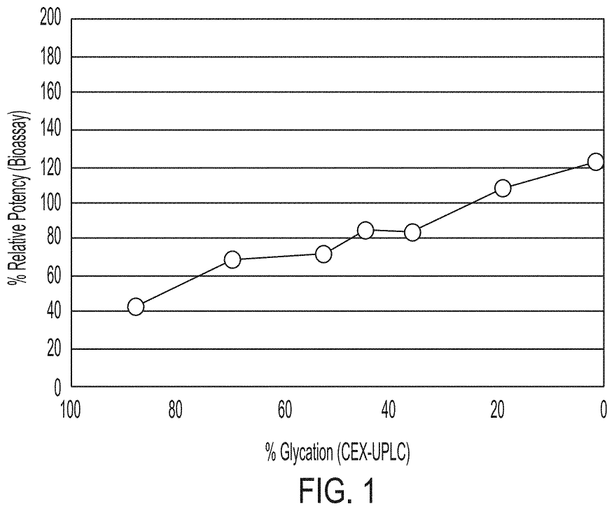

A specific mAb1-derived acidic peak, comprising approximately 50% of the total mAb1-derived acidic charge variants, was observed by CEX-UPLC analysis. This peak area, detected by CEX-UPLC, decreased following incubation at 45° C. for 28 days. This peak was identified as a mAb1 variant containing glycation at HC Lys98 within CDR3 of the MUC16 binding arm of the bispecific antibody. Along with the decrease in the level of the glycated form of mAb1, an increase in potency by bioassay was observed (see FIG. 1). Although the Lys98 de-glycation reaction readily occurs in solution, the level of glycation in the lyophilized state remained unchanged when the formulation was stored at 5° C. for at least 12 months. Additionally, the potency remained unchanged when the lyophilized formulation was stored at 5° C. for up to 12 months. Thus, a lyophilized formulation was chosen as the formulation for mAb1.

### Surfactant/Organic Co-Solvent Selection

The effect of the surfactants, polysorbate 20 and polysorbate 80, on the agitation stress stability and thermal stability of 5 mg/mL mAb1 was examined in liquid formulations (10% sucrose was present in the formulations containing surfactant). Polysorbate 20 and polysorbate 80 at concentrations of 0-0.1% (w/v) were tested for the ability to stabilize mAb1, (see Tables 3 and 4, below). Polysorbate 20 and polysorbate 80 were both able to stabilize mAb1 to agitation stress when present at levels of 0.01% or higher, as shown in Table 3. When surfactant was not included in the formulation, increases in HMW species of up to 1.9% were observed. No increases in HMW species were observed when 0.01% polysorbate 20 or polysorbate 80 was included in the formulation.

Concentrations of 0.05% polysorbate 20 and 0.05% polysorbate 80 were chosen to examine the impact of thermal stress on mAb1 stability. Following incubation for 28 days at 45° C., approximately 0.5% less HMW species and 1.6% more mAb1 monomer were observed for the formulation containing 0.05% polysorbate 20, compared to the formulation containing 0.05% polysorbate 80, as shown in Table 4. Furthermore, mAb1 exhibited an improved stability profile in the presence of 0.05% polysorbate 20, as determined by CEX-UPLC analysis following incubation at 45° C. for 28 days. Relative to mAb1 formulated in the presence of 0.05% polysorbate 80 (see Table 4), mAb1 in the presence of 0.05% polysorbate 20 showed (1) a decrease of approximately 5% in the formation of acidic species, (2) an approximately 6% decrease in the loss of main charge variant forms, and (3) approximately equivalent changes in levels of glycated species. 0.05% polysorbate 20 was chosen as the surfactant for the mAb1 drug product formulation because it sufficiently stabilized the protein to agitation stress.

### Thermal Stabilizer/Cryoprotectant

Stabilizers, such as sucrose, can sometimes be added to liquid and lyophilized antibody formulations to increase the thermal stability and the stability of the protein to freeze/thaw stress. Inclusion of sucrose in the mAb1 formulation was required to stabilize mAb1 to freeze/thaw stress. In the absence of sucrose, the level of HMW species increased by 0.4% after mAb1 was subjected to four freezing and thawing cycles (see Table 5). Addition of 10% sucrose to the formulation stabilized mAb1 drug substance to four freeze/thaw cycles (see Table 5).

Sucrose was also necessary as a cryoprotectant to stabilize mAb1 during the lyophilization process. Following reconstitution of lyophilized mAb1 formulations, the relative amount of HMW species increased 0.2% in the absence of sucrose but exhibited no appreciable changes when the formulation contained 10% sucrose, as shown in Table 6. When lyophilized mAb1 was incubated at 50° C. for 28 days in the absence of sucrose, the level of HMW species increased by 5.9% and the level of monomer decreased by 7.1%, as determined by SE-UPLC, (see Table 7). Moreover, following incubation of mAb1 at 50° C. in the absence of sucrose, the level of acidic species decreased by 13.8%, the level of basic species increased by 14.3%, and the level of the glycated species decreased by 14.7%, as determined by CEX-UPLC (see Table 7). However, in the presence of 10% sucrose, no appreciable changes in the levels of molecular weight variants or charge variants were observed, as shown in Table 7.

Ten percent sucrose had positive impacts to freeze/thaw stability, thermal stability, and stability of mAb1 during the lyophilization process. Ten percent sucrose also is used as a bulking agent during lyophilization. Therefore, 10% sucrose was chosen as the stabilizer for development of the lyophilized mAb1 formulation.

### Summary of Selected Formulation Components

mAb1 exhibited maximal stability when formulated in the presence of 10 mM histidine, 0.050% polysorbate 20, and 10% sucrose at pH 6.0. The main degradation pathways identified during the development of the mAb1 liquid formulation were the formation of HMW species and charge variants. An observed charge variant of particular interest is the glycation of HC-CDR3-Lys98. The level of glycated mAb1 has been demonstrated to be unchanged following incubation of lyophilized mAb1 under stress conditions and real-time storage conditions. The information gained during the liquid formulation development and lyophilization feasibility studies formed the basis for developing a formulation for lyophilized drug product suitable for clinical applications. To enable reconstitution of lyophilized drug product to a concentration of 2 mg/mL mAb1 for IV administration, formulated drug substance containing 2 mg/mL mAB1, 10 mM histidine, pH 6.0, 10% (w/v) sucrose, and 0.05% (w/v) polysorbate 20 was developed.

TABLE 2
Effect of Buffer and pH on the Stability of 2 mg/mL mAb1 Incubated at 45° C. for 28 Days

| | |
|---|---|
| Formulation | 2 mg/mL mAb1 and 10 mM Buffer |
| Fill Volume | 0.4 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{a} | | | CEX-UPLC^{a} | | | |
| | | in OD at | | Recovered | % | % | % | % | % | % | % |
| pH/Buffer | Appearance | 405 nm) | pH | by RP-UPLC | HMW | Native | LMW | Acidic | Main | Basic | Glycated |
| pH 4.5, | Pass | 0.00 | 4.6 | 104 | 0.8 | −1.6 | 0.8 | −14.5 | 8.5 | 6.0 | −19.6 |
| Acetate | | | | | | | | | | | |
| pH 5.0, | Pass | 0.00 | 5.1 | 101 | 1.0 | −1.4 | 0.3 | −11.0 | 9.6 | 1.5 | −21.6 |
| Acetate | | | | | | | | | | | |
| pH 5.5, | Pass | 0.00 | 5.7 | 100 | 1.4 | −1.6 | 0.3 | −7.8 | 9.4 | −1.6 | −22.0 |
| Acetate | | | | | | | | | | | |
| pH 5.5, | Pass | 0.00 | 5.6 | 99 | 1.0 | −1.4 | 0.4 | −13.3 | 4.1 | 9.2 | −22.1 |
| Histidine | | | | | | | | | | | |
| pH 6.0, | Pass | 0.00 | 6.1 | 102 | 0.7 | −0.9 | 0.2 | −9.2 | 5.8 | 3.4 | −23.2 |
| Histidine | | | | | | | | | | | |
| pH 6.5, | Pass | 0.00 | 6.6 | 102 | 0.9 | −1.1 | 0.3 | −4.8 | 3.7 | 1.1 | −22.5 |
| Histidine | | | | | | | | | | | |
| pH 6.5, | Pass | 0.00 | 6.4 | 100 | 3.8 | −4.3 | 0.5 | 1.8 | −10.1 | 8.3 | −18.2 |
| Phosphate | | | | | | | | | | | |
| pH 7.0, | Pass | 0.01 | 6.9 | 95 | 3.7 | −4.5 | 0.8 | 10.5 | −14.5 | 4.0 | −19.1 |
| Phosphate | | | | | | | | | | | |
| pH 7.5, | Fail^{b} | 0.08 | 7.4 | 91 | 5.3 | −7.2 | 1.8 | −19.5 | 12.9 | 6.6 | ND^{c} |
| Phosphate | | | | | | | | | | | |

^{a}Reported as a change in purity relative to the starting material. The starting material (no incubation) contains ≥97.8% native peak by SE-UPLC and ≥42.6% main peak by CEX-UPLC in all formulations.
^{b}Sample was cloudy with no visible particles.
^{c}Not determined due to extensive degradation
CEX, cation exchange;
DS, drug substance;
FDG, Formulation Development Group;
HMW, high molecular weight;
LMW, low molecular weight;
ND, not determined;
OD, optical density;
RP, reversed-phase;
SE, size-exclusion;
UPLC, ultra performance liquid chromatography

TABLE 3
Effect of Surfactant Concentration on the Stability of 5 mg/mL mAb1 Following Agitation (120 min of Vortexing)

| | |
|---|---|
| Formulation | 5 mg/mL mAb1, 10 mM histidine, pH 6.0 |
| Fill Volume | 0.4 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{a} | | | CEX-UPLC^{a} | | | |
| Organic Co-solvent/ | Color and | in OD at | | Recovered | % | % | % | % | % | % | % |
| Surfactant/Sucrose | Appearance | 405 nm) | pH | by RP-UPLC | HMW | Native | LMW | Acidic | Main | Basic | Glycated |
| No co-solvent/sucrose | Pass | 0.02 | 6.0 | 99 | 1.3 | −1.3 | 0.0 | −0.1 | 0.1 | 0 | −0.8 |
| 10% (w/v) sucrose | Pass | 0.02 | 6.0 | 100 | 1.9 | −1.9 | 0.0 | 1.1 | −0.8 | −0.2 | −0.4 |
| 0.01% (w/v) | Pass | 0.01 | 6.0 | 102 | 0.0 | 0.0 | 0.0 | 0.1 | 1.3 | −1.5 | 0.0 |
| polysorbate 20 and | | | | | | | | | | | |
| 10% (w/v) sucrose | | | | | | | | | | | |
| 0.05% (w/v) | Pass | 0.01 | 6.0 | 103 | 0.0 | 0.0 | 0.0 | −0.4 | 0.5 | −0.1 | 0.7 |
| polysorbate 20 and | | | | | | | | | | | |
| 10% (w/v) sucrose | | | | | | | | | | | |
| 0.1% (w/v) | Pass | 0.01 | 6.0 | 103 | 0.0 | 0.0 | 0.0 | −0.2 | −1.2 | 1.3 | −0.1 |
| polysorbate 20 and | | | | | | | | | | | |
| 10% (w/v) sucrose | | | | | | | | | | | |
| 0.01% (w/v) | Pass | 0.00 | 6.0 | 105 | 0.0 | 0.0 | 0.0 | −1.1 | 0.4 | 0.7 | 1.0 |
| polysorbate 80 and | | | | | | | | | | | |
| 10% (w/v) sucrose | | | | | | | | | | | |
| 0.05% (w/v) | Pass | 0.00 | 6.0 | 104 | 0.0 | 0.0 | 0.0 | 0.5 | −1.7 | 1.2 | 0.2 |
| polysorbate 80 and | | | | | | | | | | | |
| 10% (w/v) sucrose | | | | | | | | | | | |
| 0.1% (w/v) | Pass | 0.00 | 6.0 | 105 | 0.0 | 0.0 | 0.0 | 0.9 | −0.6 | −0.3 | 0.2 |
| polysorbate 80 and | | | | | | | | | | | |
| 10% (w/v) sucrose | | | | | | | | | | | |

^{a}Reported as a change in purity relative to the starting material; The starting material (no incubation) contains ≥97.7% native peak by SE-UPLC and ≥46.8% main peak by CEX-UPLC in all formulations.
CEX, cation exchange;
DS, drug substance;
HMW, high molecular weight;
LMW, low molecular weight;
OD, optical density;
RP, reversed-phase;
SE, size- exclusion;
UPLC, ultra performance liquid chromatography

TABLE 4
Evaluation of Polysorbate 20 and Polysorbate 80 Concentration: Effect of Surfactant
Concentration on the Stability of 5 mg/mL mAb1 when Incubated at 45° C. for 28 Days

| | |
|---|---|
| Formulation | 5 mg/mL mAb1, 10 mM histidine, pH 6.0 |
| Fill Volume | 0.4 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{a} | | | CEX-UPLC^{a} | | | |
| Organic Co-solvent/ | Color and | in OD at | | Recovered | % | % | % | % | % | % | % |
| Surfactant/Sucrose | Appearance | 405 nm) | pH | by RP-UPLC | HMW | Native | LMW | Acidic | Main | Basic | Glycated |
| 0.05% (w/v) | Pass | 0.02 | 6.0 | 101 | 9.3 | −10.1 | 0.7 | 1.1 | −0.2 | −0.9 | −17.6 |
| polysorbate 20 and | | | | | | | | | | | |
| 10% (w/v) sucrose | | | | | | | | | | | |
| 0.05% (w/v) | Pass | 0.01 | 6.0 | 100 | 9.8 | −11.7 | 1.9 | 6.0 | −6.5 | 0.5 | −16.1 |
| polysorbate 80 and | | | | | | | | | | | |
| 10% (w/v) sucrose | | | | | | | | | | | |

^{a}Reported as a change in purity relative to the starting material; The starting material (no incubation) contains ≥97.6% native peak by SE-UPLC and ≥46.0% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 5
Effect of 10% Sucrose on the Stability of 5 mg/mL mAb1 Following Four Freezing and Thawing Cycles

| | |
|---|---|
| Formulation | 5 mg/mL mAb1, 10 mM histidine, pH 5.5^{a} |
| Fill Volume | 0.4 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{b} | | | CEX-UPLC^{b} | | | |
| Organic Co-solvent/ | Color and | in OD at | | Recovered | % | % | % | % | % | % | % |
| Surfactant/Sucrose | Appearance | 405 nm) | pH^{a} | by RP-UPLC | HMW | Native | LMW | Acidic | Main | Basic | Glycated |
| No sucrose | Pass | 0.01 | 5.5 | 99 | 0.4 | −0.4 | 0.1 | 3.3 | −2.9 | −0.3 | −1.4 |
| 10% (w/v) sucrose | Pass | 0.00 | 5.5 | 101 | 0.0 | 0.0 | 0.0 | −0.4 | 0.3 | 0.1 | 0.4 |

^{a}For the initial formulation development, a pH of 5.5 was used. The pH was not optimized until after the initial surfactant was selected.
^{b}Reported as a change in purity relative to the starting material; The starting material (no incubation) contains ≥97.4% native peak by SE-UPLC and ≥42.8% main peak by CEX-UPLC in all formulations.
CEX, cation exchange;
DS, drug substance;
HMW, high molecular weight;
LMW, low molecular weight;
OD, optical density;
RP, reversed-phase;
SE, size-exclusion;
UPLC, ultra performance liquid chromatography

TABLE 6
Stabilization of mAb1 During Lyophilization by Inclusion of 10% Sucrose

| | |
|---|---|
| Pre-lyophilized | 5 mg/mL mAb1, 10 mM histidine, pH 6.0, 0.05% (w/v) polysorbate 20 |
| Formulation | |
| Reconstituted | 5 mg/mL mAb1, 10 mM histidine, pH 6.0, 0.05% (w/v) polysorbate 20 |
| Formulation | |
| Fill Volume | 0.5 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{c} | | | CEX-UPLC^{c} | | | |
| | Color and | in OD at | | Recovered | % | % | % | % | % | % | % |
| Thermal Stabilizer | Appearance | 405 nm) | pH | by RP-UPLC | HMW | Native | LMW | Acidic | Main | Basic | Glycated |
| No sucrose | Pass | 0.01 | 6.1 | 105 | 0.2 | −0.1 | −0.1 | −0.4 | 0.2 | 0.1 | 0.2 |
| 10% (w/v) sucrose | Pass | 0.02 | 6.1 | 104 | 0.0 | 0.1 | −0.1 | −0.6 | 0.5 | 0.1 | 0.2 |

(mAb1 was reconstituted and analyzed immediately after lyophilization was complete)
^{c}Reported as a change in purity relative to the starting material; The starting material (no lyophilization) contains ≥98.7% native peak by SE-UPLC and ≥45.1% main peak by CEX-UPLC in both formulations.
CEX, cation exchange;
DS, drug substance;
HMW, high molecular weight;
LMW, low molecular weight;
OD, optical density;
RP, reversed-phase;
SE, size- exclusion;
UPLC, ultra performance liquid chromatography

TABLE 7
Effect of the Presence of 10% Sucrose on the Stability of Lyophilized mAb1 DP Incubated at 50° C. for 28 Days

| | |
|---|---|
| Pre-lyophilized | 5 mg/mL mAb1, 10 mM histidine, pH 6.0, 0.05% (w/v) polysorbate 20 |
| Formulation | |
| Reconstituted | 5 mg/mL mAb1, 10 mM histidine, pH 6.0, 0.05% (w/v) polysorbate 20 |
| Formulation | |
| Fill Volume | 0.5 mL |
| Container/Closure | 2 mL Type 1 borosilicate glass vial with a FluorTec ®-coated 4432/50 butyl rubber stopper |

| | | Turbidity | | % Total | Change in Purity by | | | Change in Charge Variants by | | | |
|---|---|---|---|---|---|---|---|---|---|---|---|
| | | (Increase | | mAb1 | SE-UPLC^{a} | | | CEX-UPLC^{a} | | | |
| | Color and | in OD at | | Recovered | % | % | % | % | % | % | % |
| Thermal Stabilizer | Appearance | 405 nm) | pH | by RP-UPLC | HMW | Native | LMW | Acidic | Main | Basic | Glycated |
| No sucrose | Pass | 0.00 | 6.1 | 94 | 5.9 | −7.1 | 1.1 | −13.8 | −0.5 | 14.3 | −14.7 |
| 10% (w/v) sucrose | Pass | 0.01 | 6.1 | 98 | 0.0 | −0.3 | 0.3 | −1.1 | 0.8 | 0.4 | −0.9 |

^{a}Reported as a change in purity relative to the starting material; The starting material (no lyophilization) contains ≥98.7% native peak by SE-UPLC and ≥45.1% main peak by CEX-UPLC in both formulations.
CEX, cation exchange;
DS, drug substance;
HMW, high molecular weight;
LMW, low molecular weight;
OD, optical density;
RP, reversed-phase;
SE, size- exclusion;
UPLC, ultra performance liquid chromatography

### Example 2: Storage and Stress Stability of Formulations

Studies to evaluate the storage, accelerated stability, and stress stability (agitation) of liquid, lyophilized, and reconstituted formulations of mAb1 drug product were undertaken. Results from the analysis of mAb1 lyophilized drug product show that the mAb1 drug product was physically and chemically stable when stored at 5° C. for at least 36 months (see Tables 8, 10 and 12). No appreciable change in the physical or chemical stability was detected in any of the monitored attributes. Results from the analysis of the mAb1 lyophilized drug product following incubation under accelerated conditions are provided in Tables 9, 11 and 13. Following incubation for 3 months at 37° C., no appreciable change in the physical or chemical stability of mAb1 was detected in any of the monitored attributes. Similarly, no appreciable change in the physical or chemical stability was detected in any of the monitored attributes after incubation for 6 months at 25° C./60% relative humidity (RH). Following incubation for 3 months at 50° C., minor increases in HMW species (0.3%) and basic species (7.6% by CEX-UPLC and 4.0% by icIEF) were observed. mAb1 maintained potency, as determined by bioassay analysis after incubation under the accelerated conditions.

TABLE 8
Research Stability of mAb1 Lyophilized Drug Product, Stored at 5° C.

| Pre-lyophilized | 2 mg/mL mAb1, 10 mM histidine, pH 6.0, 10% (w/v) sucrose, | | | | | | | | |
|---|---|---|---|---|---|---|---|---|---|
| Formulation | 0.05% (w/v) polysorbate 20 | | | | | | | | |
| Fill Volume | 2.5 mL | | | | | | | | |
| Container/Closure | 5 mL Type 1 borosilicate glass vial with West V10-F597W | | | | | | | | |
| | 4432/50 B2-TR stopper | | | | | | | | |
| | Length of Storage at 5° C. (months) | | | | | | | | |
| Assay^{a} | 0 | 1 | 3^{b} | 6 | 9 | 12 | 18 | 24 | 36 |

Analysis of Lyophilized Drug Product

| | | | | | | | | | |
|---|---|---|---|---|---|---|---|---|---|
| Cake Appearance | Pass | Pass | Pass | Pass | Pass | Pass | Pass | Pass | Pass |
| % Moisture | 0.68 | NR | 0.81 | 0.81 | NR | 0.75 | NR | 0.76 | 1.15 |
| Reconstitution Time (seconds) | 62 | 55 | 55 | 54 | 55 | 54 | 56 | 55 | 33 |

Analysis of Reconstituted Drug Product^{c}

| Color and Appearance | | Pass | Pass | Pass | Pass | Pass | Pass | Pass | Pass | Pass |
|---|---|---|---|---|---|---|---|---|---|---|
| Turbidity (Increase in OD at 405 nm) | | 0.00 | 0.00 | 0.00 | 0.00 | 0.00 | 0.00 | 0.00 | 0.00 | 0.00 |
| pH | | 6.0 | 6.0 | 6.0 | 6.1 | 6.0 | 6.1 | 6.1 | 6.0 | 6.1 |
| Particulate Analysis | 2-10 μm | 200 | NR | 84 | 424 | NR | 232 | NR | 759 | 429 |
| by MFI (particles/ | ≥10 μm | 10 | NR | 7 | 10 | NR | 17 | NR | 30 | 21 |
| container) | ≥25 μm | 5 | NR | 2 | 2 | NR | 1 | NR | 5 | 5 |
| % Protein Recovered by RP-UPLC | | 100 | 99 | 104 | 101 | 100 | 100 | 103 | 101 | 96 |
| Purity by | Non-reduced; | 100 | NR | 100 | 100 | NR | 100 | NR | 100 | 100 |
| MCE-SDS | % main peak | | | | | | | | | |
| | Reduced; | 100 | NR | 100 | 100 | NR | 100 | NR | 100 | 100 |
| | % heavy + | | | | | | | | | |
| | light chain | | | | | | | | | |
| Purity by | % HMW | 0.6 | 0.5 | 0.6 | 0.6 | 0.6 | 0.6 | 0.6 | 0.6 | 0.5 |
| SE-UPLC | % Native | 99.0 | 99.1 | 99.1 | 99.0 | 99.0 | 99.0 | 99.0 | 98.8 | 99.1 |
| | % LMW | 0.4 | 0.4 | 0.4 | 0.5 | 0.4 | 0.5 | 0.4 | 0.6 | 0.4 |
| Charge Variant | % Acidic | 50.2 | 51.9 | 50.5 | 51.8 | 51.3 | 52.1 | 51.6 | 51.7 | 51.3 |
| Analysis by | % Main | 47.3 | 45.5 | 46.9 | 44.5 | 45.5 | 44.8 | 44.9 | 45.0 | 46.1 |
| CEX-UPLC | % Basic | 2.5 | 2.7 | 2.6 | 3.7 | 3.2 | 3.1 | 3.5 | 3.4 | 2.6 |
| | % Glycated | 28.4 | 27.9 | 28.5 | 27.6 | 27.4 | 27.6 | 27.5 | 27.1 | 26.4 |
| Charge Variant | % Acidic | 47.0 | NR | 48.9 | 48.1 | NR | 48.9 | NR | 48.6 | 48.2 |
| Analysis by | % Main | 44.6 | NR | 43.2 | 43.7 | NR | 43.0 | NR | 43.8 | 43.6 |
| iCIEF | % Basic | 8.5 | NR | 8.0 | 8.2 | NR | 8.1 | NR | 7.6 | 8.3 |

| | | | | | | | | | |
|---|---|---|---|---|---|---|---|---|---|
| % Relative Potency (Bioassay) | 125 | NR | 73 | NR | NR | 89 | NR | 67 | 106 |

^{a}CEX, cation exchange; DS, drug substance; FDG, Formulation Development Group; HMW, high molecular weight; iCIEF, imaged capillary isoelectric focusing; LMW, low molecular weight; MCE, microchip capillary electrophoresis; MFI, Micro-Flow Imaging ™; NR, not required; OD, optical density; RP, reversed-phase; SDS, sodium dodecyl sulfate; SE, size-exclusion; UPLC, ultra performance liquid chromatography
^{b}Actual length of storage for these samples is 4 months
^{c}Samples were reconstituted with sterile WFI to 2 mg/mL mAb1

TABLE 9
Research Stability of mAb1 Lyophilized Drug
Product, Stored Under Accelerated Conditions

| | |
|---|---|
| Pre-lyophilized | 2 mg/mL mAb1, 10 mM histidine, pH 6.0, 10% (w/v) sucrose, |
| Formulation | 0.05% (w/v) polysorbate 20 |
| Fill Volume | 2.5 mL |
| Container/Closure | 5 mL Type 1 borosilicate glass vial with West V10-F597W |
| | 4432/50 B2-TR stopper |

| | | 25° C./60% RH Storage | | 37° C. Storage | |
|---|---|---|---|---|---|
| | No Storage | (months) | | (months) | |
| Assay | t = 0 | 3 | 6 | 1 | 3 |

Analysis of Lyophilized Drug Product

| | | | | | |
|---|---|---|---|---|---|
| Cake Appearance | Pass | Pass | Pass | Pass | Pass |
| % Moisture | 0.68 | 1.01^{a} | 0.97 | NR | 0.89 |
| Reconstitution Time (seconds) | 62 | 54 | 52 | 54 | 55 |

Analysis of Reconstituted Drug Product^{b}

| Color and Appearance | | Pass | Pass | Pass | Pass | Pass |
|---|---|---|---|---|---|---|
| Turbidity (Increase in OD at 405 nm) | | 0.00 | 0.00 | 0.00 | 0.00 | 0.00 |
| pH | | 6.0 | 6.0 | 6.0 | 6.0 | 6.0 |
| Particulate Analysis | 2-10 μm | 200 | 68^{a} | 332 | NR | 800 |
| by MFI (particles/ | ≥10 μm | 10 | 8^{a} | 5 | NR | 52 |
| container) | ≥25 μm | 5 | 3^{a} | 2 | NR | 25 |
| % Protein Recovered by RP-UPLC | | 100 | 104 | 102 | 99 | 104 |
| Purity by | Non-reduced; | 100 | 100 | 100 | NR | 100 |
| MCE-SDS | % main peak | | | | | |
| | Reduced; | 100 | 100 | 100 | NR | 100 |
| | % heavy + | | | | | |
| | light chain | | | | | |
| Purity by | % HMW | 0.6 | 0.6 | 0.6 | 0.6 | 0.6 |
| SE-UPLC | % Native | 99.0 | 99.0 | 99.0 | 99.1 | 98.9 |
| | % LMW | 0.4 | 0.4 | 0.5 | 0.4 | 0.5 |
| Charge Variant | % Acidic | 50.2 | 50.3 | 51.8 | 51.4 | 50.0 |
| Analysis by | % Main | 47.3 | 47.0 | 44.3 | 46.0 | 47.1 |
| CEX-UPLC | % Basic | 2.5 | 2.7 | 3.9 | 2.7 | 2.9 |
| | % Glycated | 28.4 | 28.4 | 27.2 | 28.0 | 28.2 |
| Charge Variant | % Acidic | 47.0 | 48.1 | 48.4 | NR | 46.8 |
| Analysis by | % Main | 44.6 | 43.2 | 42.7 | NR | 44.3 |
| iCIEF | % Basic | 8.5 | 8.7 | 9.0 | NR | 9.0 |

| | | | | | |
|---|---|---|---|---|---|
| % Relative Potency (Bioassay) | 125 | 102 | NR | NR | 93 |

^{a}Actual length of storage is 4 months
^{b}Samples were reconstituted with sterile WFI to 2 mg/mL mAb1
CEX, cation exchange;
DS, drug substance;
FDG, Formulation Development Group;
HMW, high molecular weight;
iCIEF, imaged capillary isoelectric focusing;
LMW, low molecular weight;
MCE, microchip capillary electrophoresis;
MFI, microflow imaging;
NR, not required;
OD, optical density;
RP, reversed phase;
SDS, sodium dodecyl sulfate;
SE, size exclusion;
UPLC, ultra-performance liquid chromatography

TABLE 10
Research Stability of mAb1 Lyophilized Drug Product, Stored at 5° C.

| Pre-lyophilized | 2 mg/mL mAb1, 10 mM histidine, pH 6.0, 10% (w/v) sucrose, | | | | | | | | |
|---|---|---|---|---|---|---|---|---|---|
| Formulation | 0.05% (w/v) polysorbate 20 | | | | | | | | |
| Fill Volume | 2.5 mL | | | | | | | | |
| Container/Closure | 5 mL Type 1 borosilicate glass vial with West V10-F597W | | | | | | | | |
| | 4432/50 B2-TR stopper | | | | | | | | |
| | Length of Storage at 5° C. (months) | | | | | | | | |
| Assay^{a} | 0 | 1 | 3 | 6 | 9 | 12 | 18 | 24 | 36 |

Analysis of Lyophilized Drug Product

| | | | | | | | | | |
|---|---|---|---|---|---|---|---|---|---|
| Cake Appearance | Pass | Pass | Pass | Pass | Pass | Pass | Pass | Pass | Pass |
| % Moisture | 0.44 | NR | 0.28 | 0.40 | NR | 0.52 | NR | 0.26 | 0.74 |
| Reconstitution Time (seconds) | 49 | 49 | 48 | 46 | 48 | 45 | 53 | 49 | 44 |

Analysis of Reconstituted Drug Product^{b}

| Color and Appearance | | Pass | Pass | Pass | Pass | Pass | Pass | Pass | Pass | Pass |
|---|---|---|---|---|---|---|---|---|---|---|
| Turbidity (Increase in OD at 405 nm) | | 0.00 | 0.00 | 0.00 | 0.00 | 0.00 | 0.01 | 0.00 | 0.00 | 0.01 |
| pH | | 6.0 | 6.0 | 6.0 | 6.0 | 6.0 | 6.0 | 6.0 | 6.0 | 6.0 |
| Particulate Analysis | 2-10 μm | 176 | NR | 129 | 207 | NR | 163 | NR | 193 | 710 |
| by MFI (particles/ | ≥10 μm | 7 | NR | 3 | 5 | NR | 3 | NR | 8 | 11 |
| container) | ≥25 μm | 3 | NR | 0 | 0 | NR | 2 | NR | 0 | 2 |
| % Protein Recovered by RP-UPLC | | 100 | 105 | 107 | 107 | 107 | 106 | 109 | 104 | 109 |
| Purity by | Non-reduced; | 100 | NR | 100 | 100 | NR | 100 | NR | 100 | 100 |
| MCE-SDS | % main peak | | | | | | | | | |
| | Reduced; | 100 | NR | 100 | 100 | NR | 100 | NR | 100 | 100 |
| | % heavy + | | | | | | | | | |
| | light chain | | | | | | | | | |
| Purity by | % HMW | 0.7 | 0.7 | 0.7 | 0.7 | 0.6 | 0.7 | 0.7 | 0.7 | 0.7 |
| SE-UPLC | % Native | 99.0 | 99.0 | 98.9 | 98.9 | 98.9 | 98.9 | 98.8 | 98.7 | 98.8 |
| | % LMW | 0.4 | 0.4 | 0.5 | 0.4 | 0.4 | 0.4 | 0.5 | 0.6 | 0.6 |
| Charge Variant | % Acidic | 48.8 | 48.4 | 49.1 | 49.3 | 48.3 | 49.4 | 49.3 | 49.7 | 49.6 |
| Analysis by | % Main | 47.7 | 47.9 | 46.7 | 46.6 | 47.7 | 47.0 | 47.8 | 46.2 | 47.0 |
| CEX-UPLC | % Basic | 3.5 | 3.7 | 4.3 | 4.1 | 4.0 | 3.6 | 2.9 | 4.0 | 3.5 |
| | % Glycated | 27.1 | 27.1 | 27.1 | 26.9 | 26.9 | 26.5 | 26.3 | 24.9 | 25.7 |
| Charge Variant | % Acidic | 47.5 | NR | 47.8 | 47.6 | NR | 45.6 | NR | 46.9 | 46.0 |
| Analysis by | % Main | 43.5 | NR | 43.1 | 43.0 | NR | 45.0 | NR | 43.8 | 44.9 |
| iCIEF | % Basic | 9.0 | NR | 9.1 | 9.3 | NR | 9.4 | NR | 9.4 | 9.1 |

| | | | | | | | | | |
|---|---|---|---|---|---|---|---|---|---|
| % Relative Potency (Bioassay) | 70 | NR | 139 | 110 | NR | 109 | NR | 88 | 84 |

^{a}CEX, cation exchange; DS, drug substance; FDG, Formulation Development Group; HMW, high molecular weight; iCIEF, imaged capillary isoelectric focusing; LMW, low molecular weight; MCE, microchip capillary electrophoresis; MFI, Micro-Flow Imaging ™; NR, not required; OD, optical density; RP, reversed-phase; SDS, sodium dodecyl sulfate; SE, size-exclusion; UPLC, ultra performance liquid chromatography
^{b}Samples were reconstituted with sterile WFI to 2 mg/mL mAb1

TABLE 11
Research Stability of mAb1 Lyophilized Drug
Product, Stored Under Accelerated Conditions

| | |
|---|---|
| Pre-lyophilized | 2 mg/mL mAb1, 10 mM histidine, pH 6.0, 10% (w/v) sucrose, |
| Formulation | 0.05% (w/v) polysorbate 20 |
| Fill Volume | 2.5 mL |
| Container/Closure | 5 mL Type 1 borosilicate glass vial with West V10-F597W |
| | 4432/50 B2-TR stopper |

| | | 25° C./60% RH Storage | | 37° C. Storage | |
|---|---|---|---|---|---|
| | No Storage | (months) | | (months) | |
| Assay | t = 0 | 3 | 6 | 1 | 3 |

Analysis of Lyophilized Drug Product

| | | | | | |
|---|---|---|---|---|---|
| Cake Appearance | Pass | Pass | Pass | Pass | Pass |
| % Moisture | 0.44 | 0.60 | 0.80 | NR | 0.71 |
| Reconstitution Time (seconds) | 49 | 48 | 50 | 43 | 51 |

Analysis of Reconstituted Drug Product^{a}

| Color and Appearance | | Pass | Pass | Pass | Pass | Pass |
|---|---|---|---|---|---|---|
| Turbidity (Increase in OD at 405 nm) | | 0.00 | 0.00 | 0.00 | 0.00 | 0.00 |
| pH | | 6.0 | 6.0 | 6.0 | 6.0 | 6.0 |
| Particulate Analysis | 2-10 μm | 176 | 277 | 133 | NR | 281 |
| by MFI (particles/ | ≥10 μm | 7 | 0 | 8 | NR | 0 |
| container) | ≥25 μm | 3 | 0 | 3 | NR | 0 |
| % Protein Recovered by RP-UPLC | | 100 | 106 | 108 | 105 | 105 |
| Purity by | Non-reduced; | 100 | 100 | 100 | 100 | 100 |
| MCE-SDS | % main peak | | | | | |
| | Reduced; | 100 | 100 | 100 | 100 | 100 |
| | % heavy + | | | | | |
| | light chain | | | | | |
| Purity by | % HMW | 0.7 | 0.7 | 0.7 | 0.7 | 0.7 |
| SE-UPLC | % Native | 99.0 | 98.9 | 98.9 | 98.9 | 98.8 |
| | % LMW | 0.4 | 0.5 | 0.5 | 0.5 | 0.5 |
| Charge Variant | % Acidic | 48.8 | 49.0 | 49.1 | 48.1 | 48.5 |
| Analysis by | % Main | 47.7 | 46.7 | 46.3 | 48.0 | 46.6 |
| CEX-UPLC | % Basic | 3.5 | 4.4 | 4.6 | 3.9 | 4.9 |
| | % Glycated | 27.1 | 27.0 | 26.7 | 27.0 | 26.5 |
| Charge Variant | % Acidic | 47.5 | 46.5 | 47.4 | 47.7 | 46.1 |
| Analysis by | % Main | 43.5 | 44.6 | 43.1 | 42.8 | 44.5 |
| iCIEF | % Basic | 9.0 | 9.0 | 9.4 | 9.5 | 9.3 |

| | | | | | |
|---|---|---|---|---|---|
| % Relative Potency (Bioassay) | 70 | 121 | 95 | NR | 111 |

^{a}Samples were reconstituted with sterile WFI to 2 mg/mL mAb1
CEX, cation exchange;
DS, drug substance;
FDG, Formulation Development Group;
HMW, high molecular weight;
iCIEF, imaged capillary isoelectric focusing;
LMW, low molecular weight;
MCE, microchip capillary electrophoresis;
MFI, Micro-Flow Imaging ™;
NR, not required;
OD, optical density;
RP, reversed-phase;
SDS, sodium dodecyl sulfate;
SE, size-exclusion;
UPLC, ultra performance liquid chromatography

TABLE 12
Research Stability of mAb1 50 mg Lyophilized Drug Product, Stored at 5° C.

| Pre-lyophilized | 20 mg/mL mAb1, 10 mM histidine, pH 6.0, 10% (w/v) sucrose, | | | | | | | | |
|---|---|---|---|---|---|---|---|---|---|
| Formulation | 0.05% (w/v) polysorbate 20 | | | | | | | | |
| Fill Volume | 2.5 mL | | | | | | | | |
| Container/Closure | 5 mL Type 1 borosilicate glass vial with West V10-F597W | | | | | | | | |
| | 4432/50 B2-TR stopper | | | | | | | | |
| | Length of Storage at 5° C. (months) | | | | | | | | |
| Assay^{a} | 0 | 1 | 3 | 6 | 9 | 12 | 18 | 24 | 36^{b} |

Analysis of Lyophilized Drug Product

| | | | | | | | | | |
|---|---|---|---|---|---|---|---|---|---|
| Cake Appearance | Pass | Pass | Pass | Pass | Pass | Pass | Pass | Pass | Pass |
| % Moisture | 0.74 | NR | NR | 0.99 | NR | 1.16 | NR | 1.56 | 1.18 |
| Reconstitution Time (seconds) | 49 | 44 | 60 | 66 | 55 | 53 | 48 | 47 | 29 |

Analysis of Reconstituted Drug Product^{c}

| Color and Appearance | | Pass | Pass | Pass | Pass | Pass | Pass | Pass | Pass | Pass |
|---|---|---|---|---|---|---|---|---|---|---|
| Turbidity (Increase in OD at 405 nm) | | 0.00 | 0.00 | 0.00 | 0.00 | 0.00 | 0.00 | 0.00 | 0.00 | 0.00 |
| pH | | 5.9 | 5.9 | 5.9 | 5.9 | 5.9 | 5.9 | 5.9 | 6.0 | 6.0 |
| Particulate Analysis | 2-10 μm | 259 | NR | NR | 1274 | NR | 2707 | NR | 2343 | NA |
| by MFI (particles/ | ≥10 μm | 30 | NR | NR | 28 | NR | 48 | NR | 21 | NA |
| container) | ≥25 μm | 5 | NR | NR | 3 | NR | 4 | NR | 1 | NA |
| % Protein Recovered by RP-UPLC | | 100 | 100 | 103 | 101 | 100 | 99 | 99 | 91 | 108 |
| Purity by | Non-reduced; | 100 | NR | NR | 100 | NR | 100 | NR | 100 | 100 |
| MCE-SDS | % main peak | | | | | | | | | |
| | Reduced; | 100 | NR | NR | 100 | NR | 100 | NR | 100 | 100 |
| | % heavy + | | | | | | | | | |
| | light chain | | | | | | | | | |
| Purity by | % HMW | 0.8 | 0.8 | 0.9 | 0.8 | 0.9 | 0.9 | 0.9 | 0.8 | 0.9 |
| SE-UPLC | % Native | 98.8 | 98.9 | 98.6 | 98.8 | 98.7 | 98.7 | 98.7 | 98.8 | 98.6 |
| | % LMW | 0.4 | 0.4 | 0.5 | 0.4 | 0.5 | 0.4 | 0.5 | 0.4 | 0.5 |
| Charge Variant | % Acidic | 47.7 | 48.0 | 48.8 | 48.3 | 49.5 | 48.3 | 48.1 | 49.7 | 47.7 |
| Analysis by | % Main | 47.1 | 47.3 | 45.9 | 46.8 | 42.5 | 45.3 | 46.2 | 43.8 | 46.7 |
| CEX-UPLC | % Basic | 5.1 | 4.7 | 5.3 | 4.8 | 8.0 | 6.4 | 5.7 | 6.5 | 5.5 |
| | % Glycated | 24.4 | 24.0 | 24.5 | 24.2 | 21.3 | 22.1 | 23.2 | 22.2 | 23.0 |
| Charge Variant | % Acidic | 45.1 | NR | NR | 44.8 | NR | 47.8 | NR | 47.7 | 47.2 |
| Analysis by | % Main | 46.0 | NR | NR | 46.5 | NR | 45.5 | NR | 45.1 | 45.3 |
| iCIEF | % Basic | 8.8 | NR | NR | 8.7 | NR | 6.7 | NR | 7.2 | 7.4 |

| | | | | | | | | | |
|---|---|---|---|---|---|---|---|---|---|
| % Relative Potency (Bioassay) | 98 | NR | NR | 104 | NR | 104 | NR | 91 | 118 |

^{a}CEX, cation exchange; DS, drug substance; FDG, Formulation Development Group; HMW, high molecular weight; iCIEF, imaged capillary isoelectric focusing; LMW, low molecular weight; MCE, microchip capillary electrophoresis; MFI, Micro-Flow Imaging ™; NA, not available; NR, not required; OD, optical density; RP, reversed-phase; SDS, sodium dodecyl sulfate; SE, size-exclusion; UPLC, ultra performance liquid chromatography
^{b}The 36 months MFI data not available due to instrument failure and insufficient availability of backup samples.
^{c}Samples were reconstituted with sterile WFI to 20 mg/mLmAb1

TABLE 13
Research Stability of mAb1 50 mg Lyophilized Drug
Product, Stored Under Accelerated Conditions

| | |
|---|---|
| Pre-lyophilized | 20 mg/mL mAb1, 10 mM histidine, pH 6.0, 10% (w/v) sucrose, |
| Formulation | 0.05% (w/v) polysorbate 20 |
| Fill Volume | 2.5 mL |
| Container/Closure | 5 mL Type 1 borosilicate glass vial with West V10-F597W |
| | 4432/50 B2-TR stopper |

| | No | 25° C./60% RH Storage | 50° C. Storage | |
|---|---|---|---|---|
| | Storage | (months) | (months) | |
| Assay^{a} | t = 0 | 6 | 1 | 3 |

Analysis of Lyophilized Drug Product

| | | | | |
|---|---|---|---|---|
| Cake Appearance | Pass | Pass | Pass | Pass |
| % Moisture | 0.74 | 1.00 | NR | 1.00 |
| Reconstitution Time (seconds) | 49 | 48 | 55 | 62 |

Analysis of Reconstituted Drug Product^{b}

| Color and Appearance | | Pass | Pass | Pass | Pass |
|---|---|---|---|---|---|
| Turbidity (Increase in OD at 405 nm) | | 0.00 | 0.00 | 0.00 | 0.00 |
| pH | | 5.9 | 5.9 | 5.9 | 5.9 |
| Particulate Analysis | 2-10 μm | 259 | 1227 | NR | 11225 |
| by MFI (particles/ | ≥10 μm | 30 | 36 | NR | 429 |
| container) | ≥25 μm | 5 | 9 | NR | 64 |
| % Protein Recovered by RP-UPLC | | 100 | 99 | 100 | 99 |
| Purity by | Non-reduced; | 100 | 100 | NR | 100 |
| MCE-SDS | % main peak | | | | |
| | Reduced; | 100 | 100 | NR | 100 |
| | % heavy + | | | | |
| | light chain | | | | |
| Purity by | % HMW | 0.8 | 1.1 | 0.9 | 1.1 |
| SE-UPLC | % Native | 98.8 | 98.4 | 98.7 | 98.3 |
| | % LMW | 0.4 | 0.5 | 0.4 | 0.5 |
| Charge Variant | % Acidic | 47.7 | 47.4 | 44.5 | 43.9 |
| Analysis by | % Main | 47.1 | 46.9 | 47.2 | 43.4 |
| CEX-UPLC | % Basic | 5.1 | 5.7 | 8.4 | 12.7 |
| | % Glycated | 24.4 | 23.7 | 22.3 | 20.5 |
| Charge Variant | % Acidic | 45.1 | 48.4 | NR | 48.4 |
| Analysis by | % Main | 46.0 | 45.3 | NR | 38.8 |
| iCIEF | % Basic | 8.8 | 6.3 | NR | 12.8 |

| | | | | |
|---|---|---|---|---|
| % Relative Potency (Bioassay) | 98 | 118 | NR | NR |

^{a}CEX, cation exchange; DS, drug substance; FDG, Formulation Development Group; HMW, high molecular weight; iCIEF, imaged capillary isoelectric focusing; LMW, low molecular weight; MCE, microchip capillary electrophoresis; MFI, microflow imaging; NR, not required; OD, optical density; RP, reversed phase; SDS, sodium dodecyl sulfate; SE, size exclusion; UPLC, ultra-performance liquid chromatography
^{b}Samples were reconstituted with sterile WFI to 20 mg/mL mAb1

Additional stability studies for reconstituted mAb1 drug product were also undertaken to evaluate the stability of the reconstituted drug product when incubated at 25° C. for up to 24 hours, as well as under stress (agitation) conditions. The lyophilized drug product (2.5 mL formulated drug substance in 5 mL Type 1 glass vials) was reconstituted to 2 mg/mL mAb1 or 20 mg/ml mAb1 with 2.3 mL WFI (to 2.5 mL final volume). Results of these stability studies are shown in Tables 14 and 15, below. Reconstituted mAb1 drug product solutions at 2 mg/mL mAb1 and 20 mg/ml mAb1 were found to be physically and chemically stable when incubated at 25° C. for 24 hours. No appreciable change in the physical or chemical stability was detected in any of the monitored attributes. These data indicate that the reconstituted drug product is stable at room temperature. Reconstituted mAb1 drug product solutions at 2 mg/mL mAb1 and 20 mg/ml mAb1 were also found to be physically and chemically stable when agitated (vortexed at ambient temperature) for 60 minutes. No appreciable change in the physical or chemical stability was detected in any of the monitored attributes.

TABLE 14
Research Stability of mAb1 Reconstituted Drug Product for IV Administration

| Formulation | 2 mg/mL mAb1, 10 mM histidine, pH 6.0, 10% (w/v) sucrose, | | | | | |
|---|---|---|---|---|---|---|
| | 0.05% (w/v) polysorbate 20 | | | | | |
| Fill Volume | 2.5 mL | | | | | |
| Container/Closure | 5 mL Type 1 borosilicate glass vial with West V10-F597W | | | | | |
| | 4432/50 B2-TR stopper | | | | | |
| | | | Agitation | | 25° C. Storage | |
| | | No Stress | (minutes) | | (hours) | |
| Assay | | t = 0 | 30 | 60 | 8 | 24 |
| Color and Appearance | | Pass | Pass | Pass | Pass | Pass |
| Turbidity (Increase in OD at 405 nm) | | 0.00 | 0.00 | 0.00 | 0.00 | 0.00 |
| pH | | 6.0 | 6.0 | 6.0 | 6.0 | 6.0 |
| Particulate | 2-10 μm | 1066 | NR | 3183 | NR | 407 |
| Analysis by MFI | ≥10 μm | 68 | NR | 60 | NR | 13 |
| (particles/mL) | ≥25 μm | 6 | NR | 9 | NR | 5 |
| % Total Protein Recovered by RP-UPLC | | 100 | 100 | 100 | 100 | 101 |
| Purity by | Non-reduced; | 100 | NR | 100 | NR | 100 |
| MCE-SDS^{a} | % main peak | | | | | |
| | Reduced; | 100 | NR | 100 | NR | 100 |
| | % heavy + | | | | | |
| | light chain | | | | | |
| Purity by | % HMW | 0.6 | 0.6 | 0.6 | 0.6 | 0.6 |
| SE-UPLC | % Native | 99.0 | 98.9 | 98.9 | 98.9 | 99.0 |
| | % LMW | 0.4 | 0.5 | 0.5 | 0.5 | 0.4 |
| Charge Variant | % Acidic | 50.0 | 50.0 | 50.1 | 50.1 | 47.9 |
| Analysis by | % Main | 45.1 | 45.1 | 45.1 | 45.2 | 46.2 |
| CEX-UPLC | % Basic | 4.9 | 4.9 | 4.8 | 4.7 | 5.9 |
| | % Glycated | 23.8 | 23.6 | 23.6 | 23.5 | 24.4 |
| Charge Variant | % Acidic | 48.4 | NR | 48.4 | NR | 48.3 |
| Analysis by | % Main | 43.6 | NR | 43.6 | NR | 43.6 |
| iCIEF | % Basic | 8.0 | NR | 8.0 | NR | 8.1 |

| | | | | | |
|---|---|---|---|---|---|
| % Relative Potency by Bioassay | 88 | NR | 92 | NR | 94 |

CEX, cation exchange;
DS, drug substance;
FDG, Formulation Development Group;
HMW, high molecular weight;
iCIEF, imaged capillary isoelectric focusing;
LMW, low molecular weight;
MCE, microchip capillary electrophoresis;
MFI, Micro-Flow Imaging ™;
NR, not required;
OD, optical density;
RP, reversed-phase;
SDS, sodium dodecyl sulfate;
SE, size-exclusion;
UPLC, ultra performance liquid chromatography

TABLE 15
Research Stability of mAb1 Reconstituted 20
mg/mL Drug Product for IV Administration

| Formulation | 20 mg/mL mAb1, 10 mM histidine, pH 6.0, 10% (w/v) sucrose, | | | | | |
|---|---|---|---|---|---|---|
| | 0.05% (w/v) polysorbate 20 | | | | | |
| Fill Volume | 2.5 mL | | | | | |
| Container/Closure | 5 mL Type 1 borosilicate glass vial with West V10-F597W | | | | | |
| | 4432/50 B2-TR stopper | | | | | |
| | | | Agitation | | 25° C. Storage | |
| | | No Stress | (minutes) | | (hours) | |
| Assay | | t = 0 | 30 | 60 | 8 | 24 |
| Color and Appearance | | Pass | Pass | Pass | Pass | Pass |
| Turbidity (Increase in OD at 405 nm) | | 0.00 | 0.00 | 0.00 | 0.00 | 0.00 |
| pH | | 6.0 | 6.0 | 6.0 | 6.0 | 6.0 |
| Particulate | 2-10 μm | 8437 | NR | 4282 | NR | 2799 |
| Analysis by MFI | ≥10 μm | 1387 | NR | 134 | NR | 345 |
| (particles/mL) | ≥25 μm | 249 | NR | 15 | NR | 74 |
| % Total Protein Recovered by RP-UPLC | | 100 | 101 | 100 | 101 | 102 |
| Purity by | Non-reduced; | 100 | NR | 100 | NR | 100 |
| MCE-SDS^{a} | % main peak | | | | | |
| | Reduced; | 100 | NR | 100 | NR | 100 |
| | % heavy + | | | | | |
| | light chain | | | | | |
| Purity by | % HMW | 0.8 | 0.8 | 0.8 | 0.8 | 0.8 |
| SE-UPLC | % Native | 98.7 | 98.7 | 98.7 | 98.7 | 98.7 |
| | % LMW | 0.5 | 0.5 | 0.5 | 0.5 | 0.5 |
| Charge Variant | % Acidic | 47.7 | 47.7 | 47.4 | 47.6 | 47.4 |
| Analysis by | % Main | 46.9 | 47.0 | 47.2 | 46.5 | 46.6 |
| CEX-UPLC | % Basic | 5.4 | 5.4 | 5.5 | 5.9 | 6.0 |
| | % Glycated | 24.1 | 24.7 | 24.4 | 24.1 | 24.5 |
| Charge Variant | % Acidic | 47.3 | NR | 47.2 | NR | 47.5 |
| Analysis by | % Main | 45.2 | NR | 45.0 | NR | 44.4 |
| iCIEF | % Basic | 7.6 | NR | 7.8 | NR | 8.1 |

| | | | | | |
|---|---|---|---|---|---|
| % Relative Potency by Bioassay | 75 | NR | 64 | NR | 88 |

CEX, cation exchange;
DS, drug substance;
FDG, Formulation Development Group;
HMW, high molecular weight;
iCIEF, imaged capillary isoelectric focusing;
LMW, low molecular weight;
MCE, microchip capillary electrophoresis;
MFI, Micro-Flow Imaging ™;
NR, not required;
OD, optical density;
RP, reversed-phase;
SDS, sodium dodecyl sulfate;
SE, size-exclusion;
UPLC, ultra performance liquid chromatography

The results from the mAb1 drug product storage and stress stability studies indicate that mAb1 is stable. The mAb1 formulation can withstand short exposures to room temperature without compromising physical or chemical stability. The mAb1 formulation is also stable when reconstituted to a concentration of 2 mg/mL or 20 mg/ml. Exposure of the reconstituted mAb1 drug product to 25° C. for up to 24 hours will not compromise the integrity of the protein, nor will agitation of the reconstituted drug product.

The reconstitution volumes for the mAb1 drug product are shown in Tables 16 and 17, below.

TABLE 16
Reconstitution Volumes, Overfill Volumes, and
Withdrawable Volumes for 5 mg/ml mAb1 Drug
Product Reconstituted for IV Administration

| | Reconstituted for | |
|---|---|---|
| Formulation Component | IV Administration | |
| mAb1 Concentration in FDS (Pre-lyophilization) | 2 | mg/mL |
| Amount of Protein per Vial | 5 | mg |
| mAb1 Concentration in Reconstituted DP | 2 | mg/mL |
| Fill Volume | 2.5 | mL |
| WFI Volume for Reconstitution | 2.3 | mL |
| Final Reconstituted Volume | 2.5 | mL |
| Overfill Volume | 0.5 | mL |
| Volume Available for Withdrawal | 2.0 | mL |

DP, drug product;
FDS, formulated drug substance;
IV, intravenous;
WFI, Water for Injection

TABLE 17
Reconstitution Volumes, Overfill Volumes, and Withdrawable Volumes
for 50 mg/mL mAb1 DP Reconstituted for IV Administration

| | Reconstituted for | |
|---|---|---|
| Formulation Component | IV Administration | |
| mAb1 Concentration in FDS (Pre-lyophilization) | 20 | mg/mL |
| Amount of Protein per Vial | 50 | mg |
| mAb1 Concentration in Reconstituted DP | 20 | mg/mL |
| Target Fill Volume | 2.5 | mL |
| WFI Volume for Reconstitution | 2.3 | mL |
| Final Reconstituted Volume | 2.5 | mL |
| mAb1 Concentration in FDS (Pre-lyophilization) | 20 | mg/mL |
| Amount of Protein per Vial | 50 | mg |
| Overfill Volume | 0.5 | mL |
| Volume Available for Withdrawal | 2.0 | mL |

DP, drug product;
FDS, formulated drug substance;
IV, intravenous;
WFI, Water for Injection

Research stability studies were performed to determine the long-term storage, accelerated stability (temperatures above storage conditions), and stress stability (40° C./75% RH, agitation, freezing and thawing) of mAb1 drug substance (180 mg/ml mAb1, 30 mM acetate, 5% w/v sucrose, pH 5.0) and formulated drug substance (50 mg/ml mAb1, 30 mM acetate, 10% w/v sucrose, 0.2% w/v polysorbate 20, pH 5.0), as discussed more fully below.

No appreciable changes in the physical or chemical stability of mAb1 drug substance were detected when stored at −80° C. and −30° C. for up to 24 months (see Tables 18 and 19). These results indicate that the mAb1 drug substance is stable for at least 24 months when stored frozen at storage conditions. Results from the research accelerated stability studies are presented in Tables 20 to 22. No appreciable changes were observed in any the monitored attributes after incubating at −20° C. for up to 6 months. An increase in protein concentration was observed by SoloVPE after incubation at 5° C. and 25° C./60% RH for up to 6 months, likely due to sample evaporation. An increase in HMW species was observed by SE-UPLC and non-reduced MCE after incubation at 5° C. and 25° C./60% RH for up to 6 months. A decrease in Region 1 (acidic species) with a concomitant increase in Region 2 (main peak) was observed by CEX-UPLC after incubation at 5° C. and 25° C./60% RH for 6 months because of the de-glycation at HC-CDR3-Lys98 of MUC16 arm. An increase in Region 1 (acidic species) with a concomitant decrease in Region 2 (main peak) was observed by iCIEF after incubation at 25° C./60% RH for 6 months likely due to deamidation. These results indicate that the mAb1 drug substance can withstand incubation at −20° C. for at least 6 months without compromising either the physical or chemical stability of the protein. Results from the research stress stability studies are presented in Tables 23 and 24 and 17. The mAb1 drug substance was physically and chemically stable when agitated (vortexed) for 10 minutes or exposed to 4 freeze/thaw cycles. An increase in protein concentration was observed by SoloVPE after incubation at 40° C./75% RH for up to 3 months, likely due to sample evaporation. An increase in color intensity was observed after incubation at 40° C./75% RH for 3 months. Increases in HMW and LMW species were observed by SE-UPLC and MCE after incubation at 40° C./75% RH for up to 3 months. A decrease in Region 1 with a concomitant increase in Region 2 was observed by CEX-UPLC after incubation at 40° C./75% RH because of the de-glycation at HC-CDR3-Lys98 of MUC16 arm. An increase in Region 3 with a concomitant decrease in Region 2 was observed by CEX-UPLC after incubation at 40° C./75% RH for 3 months. The increase in Region 3 is due to an increased basic peak eluted during the high salt elution step required for CEX-UPLC. The results from the accelerated and stressed conditions indicated that HMW, LMW, and charge variants are the major degradation pathways for the mAb1 drug substance.

TABLE 18
Stability of mAb1 Drug Substance at −80° C.

| | |
|---|---|
| Formulation | 182.0 mg/mL mAb1, 30 mM acetate, 5% (w/v) sucrose, pH 5.0 |
| Container/Closure | 5 mL gamma-irradiated polycarbonate vial with HDPE closure |

| | | Length of Storage at −80° C. (months) | | | | | | | | |
|---|---|---|---|---|---|---|---|---|---|---|
| Assay | | 0 | 1 | 3 | 6 | 9 | 12 | 18 | 24 | 36 |
| Physical Form/Condition | | LEFVP | LEFVP | LEFVP | LEFVP | LEFVP | LEFVP | LEFVP | LEFVP | |
| Clarity | | Not > I | Not > I | Not > I | Not > I | Not > I | Not > I | Not > I | Not > I | |
| Color | | Not > BY3 | Not > BY3 | Not > BY3 | Not > BY3 | Not > BY3 | Not > BY2 | Not > BY2 | Not > BY3 | |
| pH | | 5.0 | 5.0 | 5.0 | 5.0 | 5.0 | 4.9 | 4.9 | 5.0 | |
| Total Protein (mg/mL) | | 181.1 | 181.9 | 185.6 | 189.5 | 189.2 | 181.4 | 183.3 | 186.1 | |
| Potency (%) | | 126 | NR | NR | 106 | NR | 87 | NR | 80 | |
| Reduced | Purity | 97.0 | 97.5 | 97.2 | 97.2 | 97.3 | 97.1 | 97.8 | 96.9 | |
| MCE (%) | LMW | 0.4 | 0.4 | 0.4 | 0.5 | 0.2 | 0.5 | 0.2 | 0.8 | |
| Non-reduced | Purity | 97.7 | 97.8 | 97.7 | 97.6 | 96.1 | 97.0 | 97.1 | 97.4 | |
| MCE (%) | LMW | 1.8 | 1.8 | 1.7 | 1.7 | 2.8 | 2.4 | 1.7 | 1.6 | |
| SE-UPLC | Purity | 98.3 | 98.2 | 98.3 | 98.2 | 98.2 | 98.3 | 98.3 | 98.3 | |
| (%) | LMW | 0.0 | 0.0 | 0.0 | 0.0 | 0.0 | 0.0 | 0.0 | 0.0 | |
| | HMW | 1.7 | 1.9 | 1.8 | 1.8 | 1.8 | 1.7 | 1.7 | 1.7 | |
| CEX-UPLC | Region 1 | 51.5 | 51.7 | 51.4 | 51.1 | 51.7 | 52.0 | 52.2 | 51.8 | |
| (%) | Glycated | 35.8 | 34.9 | 34.5 | 35.1 | 35.0 | 34.9 | 35.0 | 34.6 | |
| | Region 2 | 44.6 | 44.2 | 44.7 | 44.9 | 43.6 | 43.4 | 43.5 | 43.6 | |
| | Region 3 | 4.0 | 4.0 | 3.9 | 4.0 | 4.7 | 4.7 | 4.3 | 4.6 | |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY2, not more intensely colored than Reference Solution BY2;
Not > BY3, not more intensely colored than Reference Solution BY3;
Not > I, not more turbid than Reference Suspension I;
NR, not required;
SE, size-exclusion;
UPLC, ultra performance liquid chromatography

TABLE 19
Stability of mAb1 Drug Substance at −30° C.

| | |
|---|---|
| Formulation | 182.0 mg/mL mAb1, 30 mM acetate, 5% (w/v) sucrose, pH 5.0 |
| Container/Closure | 5 mL gamma-irradiated polycarbonate vial with HDPE closure |

| | | Length of Storage at −30° C. (months) | | | | | | | | |
|---|---|---|---|---|---|---|---|---|---|---|
| Assay | | 0 | 1 | 3 | 6 | 9 | 12 | 18 | 24 | 36 |
| Physical Form/Condition | | LEFVP | LEFVP | LEFVP | LEFVP | LEFVP | LEFVP | LEFVP | LEFVP | |
| Clarity | | Not > I | Not > I | Not > I | Not > I | Not > I | Not > I | Not > I | Not > I | |
| Color | | Not > BY3 | Not > BY3 | Not > BY3 | Not > BY3 | Not > BY3 | Not > BY2 | Not > BY2 | Not > BY3 | |
| pH | | 5.0 | 5.0 | 5.0 | 5.0 | 5.0 | 5.0 | 4.9 | 5.0 | |
| Total Protein (mg/mL) | | 181.1 | 187.2 | 182.7 | 187.1 | 181.1 | 185.8 | 182.6 | 188.3 | |
| Potency (%) | | 126 | NR | NR | 106 | NR | 92 | NR | 105 | |
| Reduced | Purity | 97.0 | 97.7 | 97.5 | 96.9 | 97.1 | 97.1 | 97.4 | 96.6 | |
| MCE (%) | LMW | 0.4 | 0.3 | 0.3 | 0.5 | 0.3 | 0.5 | 0.2 | 0.8 | |
| Non-reduced | Purity | 97.7 | 97.8 | 97.7 | 98.1 | 97.0 | 97.5 | 95.7 | 97.6 | |
| MCE (%) | LMW | 1.8 | 1.7 | 1.7 | 1.5 | 2.3 | 2.0 | 1.8 | 1.5 | |
| SE-UPLC | Purity | 98.3 | 98.2 | 98.2 | 98.2 | 98.2 | 98.2 | 98.2 | 98.1 | |
| (%) | LMW | 0.0 | 0.0 | 0.0 | 0.0 | 0.0 | 0.0 | 0.0 | 0.0 | |
| | HMW | 1.7 | 1.8 | 1.8 | 1.8 | 1.8 | 1.8 | 1.8 | 1.9 | |
| CEX-UPLC | Region 1 | 51.5 | 51.8 | 51.4 | 51.2 | 51.5 | 51.8 | 52.2 | 51.7 | |
| (%) | Glycated | 35.8 | 34.9 | 34.7 | 35.1 | 34.9 | 34.6 | 35.1 | 34.5 | |
| | Region 2 | 44.6 | 44.3 | 44.7 | 45.0 | 43.7 | 43.3 | 43.6 | 43.9 | |
| | Region 3 | 4.0 | 3.9 | 3.9 | 3.9 | 4.7 | 4.9 | 4.3 | 4.5 | |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY2, not more intensely colored than Reference Solution BY2;
Not > BY3, not more intensely colored than Reference Solution BY3;
Not > I, not more turbid than Reference Suspension I;
NR, not required;
SE, size-exclusion;
UPLC, ultra performance liquid chromatography

TABLE 20
Stability of mAb1 Drug Substance Stored at −20° C.

| | |
|---|---|
| Formulation | 182.0 mg/mL mAb1, 30 mM acetate, 5% (w/v) sucrose, pH 5.0 |
| Container/Closure | 5 mL gamma-irradiated polycarbonate vial with HDPE closure |

| | | Length of Storage at −20° C. (months) | | | |
|---|---|---|---|---|---|
| Assay | | 0 | 1 | 3 | 6 |
| Physical Form/Condition | | LEFVP | LEFVP | LEFVP | LEFVP |
| Clarity | | Not > I | Not > I | Not > I | Not > I |
| Color | | Not > BY3 | Not > BY3 | Not > BY3 | Not > BY3 |
| pH | | 5.0 | 5.0 | 5.0 | 5.0 |
| Total Protein (mg/mL) | | 181.1 | 185.2 | 187.1 | 189.4 |
| Reduced | Purity | 97.0 | 97.4 | 97.2 | 97.1 |
| MCE (%) | LMW | 0.4 | 0.6 | 0.5 | 0.5 |
| Non-reduced | Purity | 97.7 | 97.4 | 97.5 | 97.4 |
| MCE (%) | LMW | 1.8 | 2.4 | 1.8 | 1.7 |
| SE-UPLC | Purity | 98.3 | 98.2 | 98.2 | 98.2 |
| (%) | LMW | 0.0 | 0.0 | 0.0 | 0.0 |
| | HMW | 1.7 | 1.8 | 1.8 | 1.8 |
| CEX-UPLC | Region 1 | 51.5 | 51.7 | 51.3 | 51.2 |
| (%) | Glycated | 35.8 | 34.9 | 34.1 | 35.2 |
| | Region 2 | 44.6 | 44.3 | 44.7 | 44.8 |
| | Region 3 | 4.0 | 4.0 | 4.0 | 4.0 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY3, not more intensely colored than Reference Solution BY3;
Not > I, not more turbid than Reference Suspension I;
SE, size-exclusion;
UPLC, ultra performance liquid chromatography

TABLE 21
Stability of mAb1 Drug Substance Stored at 5° C.

| Formulation | | 182.0 mg/mL mAb1, 30 mM acetate, 5% (w/v) sucrose, pH 5.0 | | | | |
|---|---|---|---|---|---|---|
| Container/Closure | | 5 mL gamma-irradiated polycarbonate vial with HDPE closure | | | | |
| | | Length of Storage at 5° C. (months) | | | | |
| Assay | | 0 | 0.5 | 1 | 3 | 6 |
| Physical Form/Condition | | LEFVP | LEFVP | LEFVP | LEFVP | LEFVP |
| Clarity | | Not > I | Not > I | Not > I | Not > I | Not > I |
| Color | | Not > BY3 | Not > BY3 | Not > BY3 | Not > BY3 | Not > BY3 |
| pH | | 5.0 | 5.0 | 5.0 | 5.0 | 5.0 |
| Total Protein (mg/mL) | | 181.1 | 183.3 | 188.4 | 187.9 | 197.5 |
| Reduced | Purity | 97.0 | 97.4 | 97.3 | 97.2 | 97.0 |
| MCE (%) | LMW | 0.4 | 0.4 | 0.3 | 0.4 | 0.6 |
| Non-reduced | Purity | 97.7 | 97.1 | 96.9 | 96.5 | 95.7 |
| MCE (%) | LMW | 1.8 | 2.3 | 2.3 | 1.8 | 2.1 |
| SE-UPLC | Purity | 98.3 | 97.9 | 97.6 | 97.0 | 96.5 |
| (%) | LMW | 0.0 | 0.0 | 0.0 | 0.0 | 0.0 |
| | HMW | 1.7 | 2.1 | 2.4 | 3.0 | 3.5 |
| CEX-UPLC | Region 1 | 51.5 | 50.0 | 50.8 | 49.3 | 47.9 |
| (%) | Glycated | 35.8 | 34.9 | 34.6 | 34.0 | 33.9 |
| | Region 2 | 44.6 | 46.0 | 45.1 | 46.4 | 48.0 |
| | Region 3 | 4.0 | 4.0 | 4.1 | 4.3 | 4.1 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY3, not more intensely colored than Reference Solution BY3;
Not > I, not more turbid than Reference Suspension I;
SE, size-exclusion;
UPLC, ultra performance liquid chromatography

TABLE 22
Stability of mAb1 Drug Substance Stored at 25° C./60% RH

| Formulation | | 182.0 mg/mL mAb1, 30 mM acetate, 5% (w/v) sucrose, pH 5.0 | | | | |
|---|---|---|---|---|---|---|
| Container/Closure | | 5 mL gamma-irradiated polycarbonate vial with HDPE closure | | | | |
| | | Length of Storage at 25° C./60% RH (months) | | | | |
| Assay | | 0 | 0.5 | 1 | 3 | 6 |
| Physical Form/Condition | | LEFVP | LEFVP | LEFVP | LEFVP | LEFVP |
| Clarity | | Not > I | Not > I | Not > I | Not > I | Not > I |
| Color | | Not > BY3 | Not > BY3 | Not > BY3 | Not > BY3 | Not > BY2 |
| pH | | 5.0 | 5.0 | 5.0 | 5.0 | 5.0 |
| Total Protein (mg/mL) | | 181.1 | 184.0 | 180.1 | 203.1 | 207.9 |
| Reduced | Purity | 97.0 | 96.7 | 97.2 | 96.8 | 96.3 |
| MCE (%) | LMW | 0.4 | 0.5 | 0.4 | 0.6 | 0.7 |
| Non-reduced | Purity | 97.7 | 96.5 | 96.1 | 94.9 | 93.7 |
| MCE (%) | LMW | 1.8 | 2.2 | 2.3 | 2.2 | 2.4 |
| SE-UPLC | Purity | 98.3 | 96.8 | 96.2 | 95.2 | 94.2 |
| (%) | LMW | 0.0 | 0.0 | 0.0 | 0.0 | 0.1 |
| | HMW | 1.7 | 3.2 | 3.8 | 4.8 | 5.7 |
| CEX-UPLC | Region 1 | 51.5 | 45.1 | 42.4 | 31.7 | 23.6 |
| (%) | Glycated | 35.8 | 31.9 | 29.0 | 19.4 | 13.5 |
| | Region 2 | 44.6 | 50.7 | 53.5 | 63.7 | 71.2 |
| | Region 3 | 4.0 | 4.2 | 4.2 | 4.6 | 5.2 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY2, not more intensely colored than Reference Solution BY2;
Not > BY3, not more intensely colored than Reference Solution BY3;
Not > I, not more turbid than Reference Suspension I;
RH, relative humidity;
SE, size-exclusion;
UPLC, ultra performance liquid chromatography

TABLE 23
Stability of mAb1 Drug Substance Stored at 40° C./75% RH

| Formulation | | 182.0 mg/mL mAb1, 30 mM acetate, 5% (w/v) sucrose, pH 5.0 | | | | |
|---|---|---|---|---|---|---|
| Container/Closure | | 5 mL gamma-irradiated polycarbonate vial with HDPE closure | | | | |
| | | Length of Storage at 40° C./75% RH (months) | | | | |
| Assay | | 0 | 0.25 | 0.5 | 1 | 3 |
| Physical Form/Condition | | LEFVP | LEFVP | LEFVP | LEFVP | LEFVP |
| Clarity | | Not > I | Not > I | Not > I | Not > I | Not > I |
| Color | | Not > BY3 | Not > BY2 | Not > BY2 | Not > BY2 | Not > BY1 |
| pH | | 5.0 | 5.0 | 5.0 | 5.0 | 5.0 |
| Total Protein (mg/mL) | | 181.1 | 187.7 | 187.7 | 191.9 | 207.1 |
| Reduced | Purity | 97.0 | 97.1 | 96.8 | 96.0 | 93.9 |
| MCE (%) | LMW | 0.4 | 0.4 | 0.5 | 0.7 | 1.4 |
| Non-reduced | Purity | 97.7 | 95.8 | 95.3 | 93.2 | 86.2 |
| MCE (%) | LMW | 1.8 | 1.9 | 2.2 | 3.7 | 5.6 |
| SE-UPLC | Purity | 98.3 | 95.0 | 92.6 | 88.4 | 78.1 |
| (%) | LMW | 0.0 | 0.0 | 0.0 | 0.2 | 0.6 |
| | HMW | 1.7 | 5.0 | 7.4 | 11.4 | 21.3 |
| CEX-UPLC | Region 1 | 51.5 | 32.1 | 22.7 | 17.6 | 18.4 |
| (%) | Glycated | 35.8 | 21.4 | 13.5 | 7.4 | 9.3 |
| | Region 2 | 44.6 | 62.5 | 70.3 | 71.7 | 55.7 |
| | Region 3 | 4.0 | 5.5 | 7.0 | 10.6 | 25.9 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY1, not more intensely colored than Reference Solution BY1;
Not > BY2, not more intensely colored than Reference Solution BY2;
Not > BY3, not more intensely colored than Reference Solution BY3;
Not > I, not more turbid than Reference Suspension I;
RH, relative humidity;
SE, size-exclusion;
UPLC, ultra performance liquid chromatography

TABLE 24
Stability of mAb1 Drug Substance - Effect of Agitation and Freeze/Thaw

| Formulation | | 182.0 mg/mL mAb1, 30 mM acetate, 5% (w/v) sucrose, pH 5.0 | | | | |
|---|---|---|---|---|---|---|
| Container/Closure | | 5 mL gamma-irradiated polycarbonate vial with HDPE closure | | | | |
| | | | Agitation | | Freezing/Thawing | |
| | | | (minutes) | | (cycles) | |
| Assay | | t = 0 | 5 | 10 | 2 | 4 |
| Physical Form/Condition | | LEFVP | LEFVP | LEFVP | LEFVP | LEFVP |
| Clarity | | Not > I | Not > I | Not > I | Not > I | Not > I |
| Color | | Not > BY3 | Not > BY3 | Not > BY3 | Not > BY2 | Not > BY2 |
| pH | | 5.0 | 5.0 | 5.0 | 5.0 | 5.0 |
| Total Protein (mg/mL) | | 181.1 | 183.9 | 183.5 | 183.7 | 187.2 |
| Reduced | Purity | 97.0 | NR | 96.7 | NR | 97.1 |
| MCE (%) | LMW | 0.4 | NR | 0.5 | NR | 0.5 |
| Non-reduced | Purity | 97.7 | NR | 97.9 | NR | 97.5 |
| MCE (%) | LMW | 1.8 | NR | 1.6 | NR | 1.9 |
| SE-UPLC | Purity | 98.3 | 98.3 | 98.3 | 98.3 | 98.2 |
| (%) | LMW | 0.0 | 0.0 | 0.0 | 0.0 | 0.0 |
| | HMW | 1.7 | 1.7 | 1.7 | 1.7 | 1.8 |
| CEX-UPLC | Region 1 | 51.5 | 51.5 | 51.4 | 49.9 | 50.6 |
| (%) | Glycated | 35.8 | 35.1 | 35.0 | 34.7 | 34.9 |
| | Region 2 | 44.6 | 44.7 | 44.6 | 46.0 | 45.3 |
| | Region 3 | 4.0 | 3.8 | 3.9 | 4.0 | 4.0 |

CEX, cation exchange;
HDPE, high density polyethylene;
HMW, high molecular weight;
LEFVP, liquid essentially free from visible particulates;
LMW, low molecular weight; MCE, microchip capillary electrophoresis;
Not > BY2, not more intensely colored than Reference Solution BY2;
Not > BY3, not more intensely colored than Reference Solution BY3;
Not > I, not more turbid than Reference Suspension I;
NR, not required;
SE, size-exclusion;
UPLC, ultra performance liquid chromatography

No appreciable changes in the physical or chemical stability of the mAb1 formulated drug substance were detected when stored at −80° C. and −30° C. for up to 24 months (see Tables 25 and 26). These results indicate that the mAb1 formulated drug substance is stable for at least 24 months when stored frozen at storage conditions. Results from the research accelerated stability studies are presented in Tables 27 to 29. No appreciable changes in the monitored attributes were observed after incubating the mAb1 formulated drug substance at −20° C. or 5° C. for up to 6 months. An increase in protein concentration was observed by SoloVPE after incubation at 25° C./60% RH for up to 6 months, likely due to sample evaporation. An increase in HMW species was observed by SE-UPLC and non-reduced MCE after incubation at 5° C. and 25° C./60% RH for up to 6 months. A decrease in Region 1 (acidic species) with a concomitant increase in Region 2 (main peak) were observed by CEX-UPLC after incubation at 25° C./60% RH for 6 months because of the de-glycation at HC-CDR3-Lys98 of MUC16 arm. An increase in Region 1 (acidic species) with a concomitant decrease in Region 2 (main peak) were observed by iCIEF after incubation at 25° C./60% RH for 6 months likely due to deamidation. These results indicate that the mAb1 formulated drug substance can withstand incubation at −20° C. for at least 6 months and at 5° C. for 3 months without compromising either the physical or chemical stability of the protein. The mAb1 formulated drug substance can also withstand short exposures to temperatures of 25° C./60% RH. Results from the research stress stability studies are presented in Tables 30 and 31. The mAb1 formulated drug substance was physically and chemically stable when agitated (vortexed) for up to 120 minutes or subjected to up to four cycles of freezing and thawing (a small increase in particles were observed by MFI after subjecting to 4 freeze/thaw cycles). An increase in protein concentration was observed by SoloVPE after incubation at 40° C./75% RH for up to 3 months, likely due to sample evaporation. Increases in HMW and LMW species were observed by SE-UPLC and MCE after incubation at 40° C./75% RH for up to 3 months. A decrease in Region 1 with a concomitant increase in Region 2 were observed by CEX-UPLC after incubation at 40° C./75% RH because of the de-glycation at HC-CDR3-Lys98 of MUC16 arm. After 2 months of incubation at 40° C./75% RH, there is a decrease in Region 1 and concomitant increase in Region 2. However, after the three-month time point the trend reverses and there is an apparent increase in Region 1 and Region 3 with concomitant decrease in Region 2. The increase in Region 1 is likely due to competing deamidation on asparagine or glutamine, whereas the increase in Region 3 is due to an increased basic peak eluted at the high salt elution step. The results from the accelerated and stressed conditions indicated that HMW, LMW, and charge variants are the major degradation pathways for the mAb1 formulated drug substance. A small increase in 2-10 μm particles was observed by MFI after 4 cycles of freezing and thawing.

TABLE 25
Stability of mAb1 Formulated Drug Substance at −80° C.

| | |
|---|---|
| Formulation | 50 mg/mL mAb1, 30 mM acetate, 10% (w/v) sucrose, 0.2% (w/v) |
| | polysorbate 20, pH 5.0 |
| Container/Closure | 5 mL gamma-irradiated polycarbonate vial with HDPE closure |

| | | Length of Storage at −80° C. (months) | | | | | | | |
|---|---|---|---|---|---|---|---|---|---|
| Assay | | 0 | 1 | 3 | 6 | 9 | 12 | 18 | 24 |
| Physical Form/Condition | | LEFVP | LEFVP | LEFVP | LEFVP | LEFVP | LEFVP | LEFVP | LEFVP |
| Clarity | | Not > I | Not > I | Not > I | Not > I | Not > I | Not > I | Not > I | Not > I |
| Color | | Not > BY4 | Not > BY4 | Not > BY4 | Not > BY4 | Not > BY4 | Not > BY4 | Not > BY4 | Not > BY5 |
| pH | | 5.0 | 4.9 | 5.0 | 5.0 | 5.0 | 4.9 | 4.9 | 5.0 |
| Total Protein (mg/mL) | | 51.6 | 52.4 | 50.1 | 51.9 | 51.0 | 49.7 | 51.2 | 52.2 |
| Potency (%) | | 92 | NR | NR | 105 | NR | 91 | NR | 90 |
| Reduced | Purity | 96.9 | 96.8 | 97.1 | 97.5 | 97.4 | 96.7 | 96.8 | 96.8 |
| MCE (%) | LMW | 0.4 | 0.5 | 0.4 | 0.5 | 0.5 | 0.5 | 0.9 | 0.9 |
| Non-reduced | Purity | 97.6 | 98.0 | 97.4 | 97.8 | 97.5 | 97.6 | 97.8 | 97.9 |
| MCE (%) | LMW | 2.0 | 1.8 | 1.9 | 1.8 | 2.2 | 2.0 | 1.8 | 1.6 |
| SE-UPLC | Purity | 98.6 | 98.6 | 98.6 | 98.6 | 98.6 | 98.6 | 98.6 | 98.4 |
| (%) | LMW | 0.0 | 0.0 | 0.0 | 0.0 | 0.0 | 0.0 | 0.0 | 0.0 |
| | HMW | 1.4 | 1.4 | 1.4 | 1.4 | 1.4 | 1.5 | 1.4 | 1.6 |
| CEX-UPLC | Region 1 | 51.8 | 51.9 | 51.5 | 51.4 | 51.9 | 52.1 | 52.4 | 50.7 |
| (%) | Glycated | 35.3 | 35.2 | 34.7 | 35.3 | 35.1 | 35.0 | 35.1 | 34.3 |
| | Region 2 | 44.5 | 44.1 | 44.7 | 44.9 | 43.7 | 43.2 | 43.5 | 44.8 |
| | Region 3 | 3.7 | 4.0 | 3.8 | 3.8 | 4.4 | 4.7 | 4.0 | 4.5 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > BY5, not more intensely colored than Reference Solution BY5;
Not > I, not more turbid than Reference Suspension I;
NR, not required;
SE, size-exclusion;
UPLC, ultra performance liquid chromatography

TABLE 26
Stability of mAb1 Formulated Drug Substance at −30° C.

| | |
|---|---|
| Formulation | 50 mg/mL mAb1, 30 mM acetate, 10% (w/v) sucrose, 0.2% (w/v) |
| | polysorbate 20, pH 5.0 |
| Container/Closure | 5 mL gamma-irradiated polycarbonate vial with HDPE closure |

| | | Length of Storage at −30° C. (months) | | | | | | | |
|---|---|---|---|---|---|---|---|---|---|
| Assay | | 0 | 1 | 3 | 6 | 9 | 12 | 18 | 24 |
| Physical Form/Condition | | LEFVP | LEFVP | LEFVP | LEFVP | LEFVP | LEFVP | LEFVP | LEFVP |
| Clarity | | Not > I | Not > I | Not > I | Not > I | Not > I | Not > I | Not > I | Not > I |
| Color | | Not > BY4 | Not > BY4 | Not > BY4 | Not > BY4 | Not > BY4 | Not > BY4 | Not > BY4 | Not > BY5 |
| pH | | 5.0 | 4.9 | 5.0 | 5.0 | 5.0 | 5.0 | 5.0 | 5.0 |
| Total Protein (mg/mL) | | 51.6 | 50.1 | 50.9 | 52.5 | 51.8 | 51.5 | 50.7 | 49.7 |
| Potency (%) | | 92 | NR | NR | 105 | NR | 85 | NR | 94 |
| Reduced | Purity | 96.9 | 96.6 | 96.5 | 97.2 | 97.4 | 97.3 | 96.8 | 96.8 |
| MCE (%) | LMW | 0.4 | 0.5 | 0.5 | 0.5 | 0.2 | 0.5 | 0.8 | 0.9 |
| Non-reduced | Purity | 97.6 | 98.0 | 97.6 | 97.4 | 97.2 | 97.6 | 97.9 | 97.8 |
| MCE (%) | LMW | 2.0 | 1.7 | 1.9 | 2.3 | 2.2 | 1.9 | 1.6 | 1.6 |
| SE-UPLC | Purity | 98.6 | 98.6 | 98.6 | 98.6 | 98.5 | 98.5 | 98.5 | 98.4 |
| (%) | LMW | 0.0 | 0.0 | 0.0 | 0.0 | 0.0 | 0.1 | 0.0 | 0.0 |
| | HMW | 1.4 | 1.4 | 1.4 | 1.4 | 1.5 | 1.5 | 1.5 | 1.6 |
| CEX-UPLC | Region 1 | 51.8 | 51.9 | 51.6 | 51.4 | 51.8 | 52.0 | 52.4 | 50.8 |
| (%) | Glycated | 35.3 | 35.1 | 34.7 | 35.1 | 34.9 | 34.9 | 35.2 | 34.3 |
| | Region 2 | 44.5 | 44.1 | 44.7 | 44.8 | 43.7 | 43.2 | 43.6 | 44.8 |
| | Region 3 | 3.7 | 4.0 | 3.8 | 3.8 | 4.5 | 4.7 | 4.1 | 4.4 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > BY5, not more intensely colored than Reference Solution BY5;
Not > I, not more turbid than Reference Suspension I;
NR, not required;
SE, size-exclusion;
UPLC, ultra performance liquid chromatography

TABLE 27
Stability of mAb1 Formulated Drug Substance Stored at −20° C.

| | |
|---|---|
| Formulation | 50 mg/mL mAb1, 30 mM acetate, 10% (w/v) sucrose, 0.2% (w/v) |
| | polysorbate 20, pH 5.0 |
| Container/Closure | 5 mL gamma-irradiated polycarbonate vial with HDPE closure |

| | | Length of Storage at −20° C. (months) | | | |
|---|---|---|---|---|---|
| Assay | | 0 | 1 | 3 | 6 |
| Physical Form/Condition | | LEFVP | LEFVP | LEFVP | LEFVP |
| Clarity | | Not > I | Not > I | Not > I | Not > I |
| Color | | Not > BY4 | Not > BY4 | Not > BY4 | Not > BY4 |
| pH | | 5.0 | 4.9 | 5.0 | 5.0 |
| Total Protein (mg/mL) | | 51.6 | 51.8 | 52.0 | 52.0 |
| Reduced | Purity | 96.9 | 97.3 | 97.4 | 97.0 |
| MCE (%) | LMW | 0.4 | 0.4 | 0.4 | 0.5 |
| Non-reduced | Purity | 97.6 | 97.9 | 97.5 | 97.6 |
| MCE (%) | LMW | 2.0 | 1.6 | 1.9 | 1.9 |
| SE-UPLC | Purity | 98.6 | 98.6 | 98.6 | 98.6 |
| (%) | LMW | 0.0 | 0.0 | 0.0 | 0.0 |
| | HMW | 1.4 | 1.5 | 1.4 | 1.5 |
| CEX-UPLC | Region 1 | 51.8 | 51.9 | 51.5 | 51.3 |
| (%) | Glycated | 35.3 | 35.2 | 34.6 | 35.1 |
| | Region 2 | 44.5 | 44.2 | 44.7 | 45.0 |
| | Region 3 | 3.7 | 3.9 | 3.8 | 3.8 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > I, not more turbid than Reference Suspension I;
SE, size-exclusion;
UPLC, ultra performance liquid chromatography

TABLE 28
Stability of mAb1 Formulated Drug Substance Stored at 5° C.

| Formulation | | 50 mg/mL mAb1, 30 mM acetate, 10% (w/v) sucrose, 0.2% (w/v) | | | | |
|---|---|---|---|---|---|---|
| | | polysorbate 20, pH 5.0 | | | | |
| Container/Closure | | 5 mL gamma-irradiated polycarbonate vial with HDPE closure | | | | |
| | | Length of Storage at 5° C. (months) | | | | |
| Assay | | 0 | 0.5 | 1 | 3 | 6 |
| Physical Form/Condition | | LEFVP | LEFVP | LEFVP | LEFVP | LEFVP |
| Clarity | | Not > I | Not > I | Not > I | Not > I | Not > I |
| Color | | Not > BY4 | Not > BY4 | Not > BY4 | Not > BY4 | Not > BY4 |
| pH | | 5.0 | 5.0 | 5.0 | 5.0 | 5.0 |
| Total Protein (mg/mL) | | 51.6 | 51.7 | 53.2 | 51.9 | 54.3 |
| Reduced | Purity | 96.9 | 97.6 | 96.4 | 97.4 | 97.2 |
| MCE (%) | LMW | 0.4 | 0.4 | 0.5 | 0.4 | 0.7 |
| Non-reduced | Purity | 97.6 | 97.6 | 97.9 | 97.4 | 97.0 |
| MCE (%) | LMW | 2.0 | 2.1 | 1.7 | 1.8 | 2.0 |
| SE-UPLC | Purity | 98.6 | 98.6 | 98.4 | 98.2 | 97.9 |
| (%) | LMW | 0.0 | 0.0 | 0.0 | 0.0 | 0.0 |
| | HMW | 1.4 | 1.4 | 1.6 | 1.8 | 2.1 |
| CEX-UPLC | Region 1 | 51.8 | 50.3 | 51.3 | 50.0 | 48.7 |
| (%) | Glycated | 35.3 | 35.1 | 34.5 | 34.7 | 33.8 |
| | Region 2 | 44.5 | 45.9 | 44.9 | 46.1 | 47.7 |
| | Region 3 | 3.7 | 3.8 | 3.8 | 3.9 | 3.7 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > I, not more turbid than Reference Suspension I;
SE, size-exclusion;
UPLC, ultra performance liquid chromatography

TABLE 29
Stability of mAb1 Formulated Drug Substance Stored at 25° C./60% RH

| Formulation | | 50 mg/mL mAb1, 30 mM acetate, 10% (w/v) sucrose, 0.2% (w/v) | | | | |
|---|---|---|---|---|---|---|
| | | polysorbate 20, pH 5.0 | | | | |
| Container/Closure | | 5 mL gamma-irradiated polycarbonate vial with HDPE closure | | | | |
| | | Length of Storage at 25° C./60% RH (months) | | | | |
| Assay | | 0 | 0.5 | 1 | 3 | 6 |
| Physical Form/Condition | | LEFVP | LEFVP | LEFVP | LEFVP | LEFVP |
| Clarity | | Not > I | Not > I | Not > I | Not > I | Not > I |
| Color | | Not > BY4 | Not > BY4 | Not > BY4 | Not > BY4 | Not > BY4 |
| pH | | 5.0 | 5.0 | 5.0 | 5.0 | 5.0 |
| Total Protein (mg/mL) | | 51.6 | 50.8 | 52.2 | 54.8 | 60.0 |
| Reduced | Purity | 96.9 | 96.3 | 96.1 | 97.0 | 96.6 |
| MCE (%) | LMW | 0.4 | 0.5 | 0.5 | 0.7 | 0.8 |
| Non-reduced | Purity | 97.6 | 97.0 | 96.9 | 96.1 | 95.7 |
| MCE (%) | LMW | 2.0 | 2.4 | 2.3 | 2.3 | 2.4 |
| SE-UPLC | Purity | 98.6 | 98.3 | 97.9 | 97.3 | 96.7 |
| (%) | LMW | 0.0 | 0.0 | 0.0 | 0.0 | 0.1 |
| | HMW | 1.4 | 1.8 | 2.2 | 2.7 | 3.2 |
| CEX-UPLC | Region 1 | 51.8 | 45.5 | 42.6 | 31.4 | 24.0 |
| (%) | Glycated | 35.3 | 32.1 | 28.1 | 19.5 | 13.8 |
| | Region 2 | 44.5 | 50.7 | 53.7 | 65.2 | 72.3 |
| | Region 3 | 3.7 | 3.8 | 3.7 | 3.4 | 3.7 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > I, not more turbid than Reference Suspension I;
RH, relative humidity;
SE, size-exclusion;
UPLC, ultra performance liquid chromatography

TABLE 30
Stability of mAb1 Formulated Drug Substance Stored at 40° C./75% RH

| Formulation | | 50 mg/mL mAb1, 30 mM acetate, 10% (w/v) sucrose, 0.2% (w/v) | | | | |
|---|---|---|---|---|---|---|
| | | polysorbate 20, pH 5.0 | | | | |
| Container/Closure | | 5 mL gamma-irradiated polycarbonate vial with HDPE closure | | | | |
| | | Length of Storage at 40° C./75% RH (months) | | | | |
| Assay | | 0 | 0.25 | 0.5 | 1 | 3 |
| Physical Form/Condition | | LEFVP | LEFVP | LEFVP | LEFVP | LEFVP |
| Clarity | | Not > I | Not > I | Not > I | Not > I | Not > II |
| Color | | Not > BY4 | Not > BY4 | Not > BY4 | Not > BY4 | Not > BY4 |
| pH | | 5.0 | 5.0 | 5.0 | 5.0 | 5.0 |
| Total Protein (mg/mL) | | 51.6 | 52.9 | 52.3 | 53.2 | 56.2 |
| Reduced | Purity | 96.9 | 97.1 | 96.8 | 96.1 | 94.7 |
| MCE (%) | LMW | 0.4 | 0.5 | 0.5 | 1.0 | 1.3 |
| Non-reduced | Purity | 97.6 | 96.4 | 96.2 | 95.2 | 90.4 |
| MCE (%) | LMW | 2.0 | 2.7 | 2.8 | 3.4 | 5.9 |
| SE-UPLC | Purity | 98.6 | 98.1 | 97.1 | 95.5 | 88.1 |
| (%) | LMW | 0.0 | 0.0 | 0.0 | 0.2 | 0.5 |
| | HMW | 1.4 | 1.9 | 2.9 | 4.4 | 11.4 |
| CEX-UPLC | Region 1 | 51.8 | 32.4 | 23.0 | 20.6 | 26.4 |
| (%) | Glycated | 35.3 | 20.6 | 13.5 | 9.7 | 13.1 |
| | Region 2 | 44.5 | 63.9 | 72.8 | 73.7 | 62.8 |
| | Region 3 | 3.7 | 3.7 | 4.2 | 5.7 | 10.8 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > I, not more turbid than Reference Suspension I;
Not > II, not more turbid than Reference Suspension II;
RH, relative humidity;
SE, size-exclusion;
UPLC, ultra performance liquid chromatography

TABLE 31
Stability of mAb1 Formulated Drug Substance Effect of Agitation and Freeze/Thaw

| Formulation | | 50 mg/mL mAb1, 30 mM acetate, 10% (w/v) sucrose, 0.2% (w/v) | | | | |
|---|---|---|---|---|---|---|
| | | polysorbate 20, pH 5.0 | | | | |
| Container/Closure | | 5 mL gamma-irradiated polycarbonate vial with HDPE closure | | | | |
| | | | Agitation | | Freezing/Thawing | |
| | | | (minutes) | | (cycles) | |
| Assay | | t = 0 | 60 | 120 | 2 | 4 |
| Physical Form/Condition | | LEFVP | LEFVP | LEFVP | LEFVP | LEFVP |
| Clarity | | Not > I | Not > I | Not > I | Not > I | Not > I |
| Color | | Not > BY4 | Not > BY4 | Not > BY4 | Not > BY4 | Not > BY4 |
| pH | | 5.0 | 5.0 | 5.0 | 5.0 | 5.0 |
| Total Protein (mg/mL) | | 51.6 | 51.7 | 52.9 | 50.3 | 51.7 |
| Reduced | Purity | 96.9 | NR | 97.7 | NR | 96.9 |
| MCE (%) | LMW | 0.4 | NR | 0.4 | NR | 0.5 |
| Non-reduced | Purity | 97.6 | NR | 97.4 | NR | 97.9 |
| MCE (%) | LMW | 2.0 | NR | 2.1 | NR | 1.8 |
| SE-UPLC | Purity | 98.6 | 98.6 | 98.6 | 98.6 | 98.6 |
| (%) | LMW | 0.0 | 0.0 | 0.0 | 0.0 | 0.0 |
| | HMW | 1.4 | 1.4 | 1.4 | 1.4 | 1.4 |
| CEX-UPLC | Region 1 | 51.8 | 51.7 | 51.7 | 50.2 | 50.7 |
| (%) | Glycated | 35.3 | 35.0 | 35.1 | 34.9 | 35.1 |
| | Region 2 | 44.5 | 44.7 | 44.5 | 46.1 | 45.4 |
| | Region 3 | 3.7 | 3.7 | 3.8 | 3.7 | 3.9 |

CEX, cation exchange;
HDPE, high density polyethylene;
HMW, high molecular weight;
LEFVP, liquid essentially free from visible particulates;
LMW, low molecular weight;
MCE, microchip capillary electrophoresis;
Not > BY4, not more intensely colored than Reference Solution BY4;
Not > I, not more turbid than Reference Suspension I;
NR, not required;
SE, size-exclusion;
UPLC, ultra performance liquid chromatography

Stability studies to evaluate the storage and accelerated stability of a liquid mAb1 drug product have been undertaken. The liquid drug product used for the storage and accelerated stability studies was manufactured by filling 2.5 mL of formulated drug substance in 6R ISO Type 1 glass vials. The liquid drug product was incubated under storage, accelerated, and stress conditions. No appreciable changes in the physical or chemical stability of the mAb1 liquid drug product were detected when stored at 5° C. for up to 12 or 24 months (see Tables 32 and 33). These results indicate that the mAb1 liquid drug product is stable for at least 12 or 24 months at the storage condition. Results from the research accelerated stability studies are presented in Tables 34 and 35. A decrease in Region 1, with a concomitant increase in Region 2, was observed by CEX-UPLC when stored at 25° C./60% RH for up to 6 months because of the de-glycation at HC-CDR3-Lys98 of MUC16 arm. An increase in Region 1, with a concomitant decrease in Region 2, was observed by iCIEF after incubation at 25° C./60% RH for up to 6 months. No appreciable changes were observed in other monitored attributes. Results from the research stressed stability studies are presented in Tables 34 to 37. The mAb1 liquid drug product was physically and chemically stable when agitated (vortexed) for 120 minutes. An increase in HMW and LMW species was observed by SE-UPLC after incubation at 40° C./75% RH for 3 months. Increases in LMW species were observed by reduced and non-reduced MCE after incubation at 40° C./75% RH for 3 months. Different trends for charge variant were observed by CEX-UPLC and iCIEF due to different sensitivities of each assay. An increase in Region 1, with a concomitant decrease in Region 2, was observed by iCIEF after incubation at 40° C./75% RH for up to 3 months, likely due to the deamidation on asparagine or glutamine. After incubation for up to 2 months at 40° C./75% RH, there is a decrease in Region 1, with a concomitant increase in Region 2 observed by CEX-UPLC. This is followed by an apparent increase in Region 1, with a concomitant decrease in Region 2, observed by CEX-UPLC at the 3 month time point, likely due to the competing deamidation reaction. A decrease in Region 3 was observed by iCIEF at the 3 month time point when incubated at 40° C./75% RH. An increase in 2-10 um particles was observed by MFI after 4 cycles of freezing and thawing. The results from the accelerated and stressed conditions indicated that HMW, LMW, and charge variants are the major degradation pathways for the mAb1 liquid drug product.

TABLE 32
Stability of 5.0 mg/ml mAb1 Liquid Drug Product Stored at 5° C.

| | |
|---|---|
| Formulation | mAb1 drug product; sterile, vialed mAb1 recombinant protein, 5.0 mg/mL, in an aqueous |
| | buffered solution, pH 5.0, containing 30 mM acetate, 10% (w/v) sucrose, and 0.2% (w/v) |
| | polysorbate 20 |
| Container Closure | 6R (5 mL) USP/Ph. Eur. Type 1 borosilicate glass vial; 20 mm FluroTec ®-coated |
| | chlorobutyl stopper; 20 mm Flip-Off ® seal |
| Storage Condition | 5° C. |

| | | Length of Storage (months) | | | | | | | | |
|---|---|---|---|---|---|---|---|---|---|---|
| Assay | | 0 | 3 | 6 | 9 | 12 | 18 | 24 | 30 | 36 |
| Physical Form/Condition | | LEFVP | LEFVP | LEFVP | LEFVP | LEFVP | LEFVP | LEFVP | | |
| Clarity | | Not > I | Not > I | Not > I | Not > I | Not > I | Not > I | Not > I | | |
| Color | | Not > BY7 | Not > BY7 | Not > BY7 | Not > BY7 | Not > BY7 | Not > BY7 | Not > BY7 | | |
| pH | | 5.0 | 5.0 | 5.0 | 5.0 | 5.0 | 5.0 | 5.0 | | |
| Total Protein (mg/mL) | | 5.2 | 5.2 | 5.2 | 5.2 | 5.2 | 5.2 | 5.2 | | |
| Potency (%) | | 135 | NR | 106 | NR | 107 | NR | 101 | | |
| Polysorbate 20 (%) | | 0.21 | NR | 0.20 | NR | 0.21 | NR | 0.21 | | |
| Reduced | Purity | 97.5 | 97.3 | 96.9 | 97.2 | 96.9 | 97.4 | 96.8 | | |
| MCE (%) | LMW | 0.2 | 0.3 | 0.5 | 0.1 | 0.6 | 0.1 | 0.3 | | |
| Non-reduced | Purity | 97.4 | 97.5 | 97.3 | 97.0 | 96.8 | 97.3 | 97.0 | | |
| MCE (%) | LMW | 2.2 | 2.0 | 2.1 | 2.5 | 2.7 | 2.1 | 2.2 | | |
| SE-UPLC | MP Purity | 98.5 | 98.6 | 98.5 | 98.5 | 98.5 | 98.4 | 98.4 | | |
| (%) | LMW | 0.0 | 0.0 | 0.0 | 0.0 | 0.0 | 0.0 | 0.0 | | |
| | HMW | 1.5 | 1.5 | 1.5 | 1.5 | 1.6 | 1.6 | 1.6 | | |
| Charge Variant | Region 1 | 41.0 | 39.6 | 38.8 | 37.7 | 37.0 | 35.9 | 34.3 | | |
| Analysis by | Glycated | 28.6 | 27.0 | 27.2 | 26.6 | 25.9 | 25.1 | 23.7 | | |
| CEX-UPLC | Region 2 | 53.9 | 55.5 | 56.6 | 57.5 | 57.6 | 59.7 | 61.8 | | |
| (%) | Region 3 | 5.2 | 4.9 | 4.7 | 4.8 | 5.5 | 4.4 | 3.8 | | |
| Particulate | ≥10 μm | 37 | 135 | 229 | NR | 1074 | NR | 1460 | | |
| Matter by Light | ≥25 μm | 0 | 0 | 0 | NR | 8 | NR | 0 | | |
| Obscuration | | | | | | | | | | |
| (#/container) | | | | | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY7, not more intensely colored than Reference Solution BY7;
Not > I, not more turbid than Reference Suspension I;
NR, not required;
Ph. Eur., European Pharmacopeia;
SE, size-exclusion;
UPLC, ultra performance liquid chromatography;
USP, United States Pharmacopeia

TABLE 33
Stability of 50.0 mg/ml mAb1 Liquid Drug Product Stored at 5° C.

| | |
|---|---|
| Formulation | mAb1 drug product; sterile, vialed mAb1 recombinant protein, 50.0 mg/mL, in an aqueous |
| | buffered solution, pH 5.0, containing 30 mM acetate, 10% (w/v) sucrose, and 0.2% (w/v) |
| | polysorbate 20 |
| Container Closure | 6R (5 mL) USP/Ph. Eur. Type 1 borosilicate glass vial; 20 mm FluroTec ®-coated |
| | chlorobutyl stopper; 20 mm Flip-Off ® seal |
| Storage Condition | 5° C. |

| | | Length of Storage (months) | | | | | | | | |
|---|---|---|---|---|---|---|---|---|---|---|
| Assay | | 0 | 3 | 6 | 9 | 12 | 18 | 24 | 30 | 36 |
| Physical Form/Condition | | LEFVP | LEFVP | LEFVP | LEFVP | LEFVP | | | | |
| Clarity | | Not > III | Not > III | Not > III | Not > III | Not > III | | | | |
| Color | | Not > BY5 | Not > BY5 | Not > BY5 | Not > BY5 | Not > BY5 | | | | |
| pH | | 5.0 | 5.0 | 5.0 | 5.0 | 5.0 | | | | |
| Total Protein (mg/mL) | | 51.0 | 49.3 | 49.5 | 49.8 | 49.4 | | | | |
| Potency (%) | | 81 | NR | 104 | NR | 130 | | | | |
| Polysorbate 20 (%) | | 0.19 | NR | 0.20 | NR | 0.20 | | | | |
| Reduced | Purity | 97.1 | 97.2 | 97.3 | 96.5 | 96.7 | | | | |
| MCE (%) | LMW | 0.3 | 0.4 | 0.1 | 0.7 | 0.8 | | | | |
| Non-reduced | Purity | 96.9 | 97.2 | 97.0 | 97.0 | 96.8 | | | | |
| MCE (%) | LMW | 2.3 | 2.0 | 2.1 | 2.1 | 2.3 | | | | |
| SE-UPLC | MP Purity | 97.9 | 97.9 | 97.8 | 97.7 | 97.6 | | | | |
| (%) | LMW | 0.0 | 0.0 | 0.0 | 0.0 | 0.0 | | | | |
| | HMW | 2.1 | 2.1 | 2.2 | 2.3 | 2.4 | | | | |
| Charge Variant | Region 1 | 39.9 | 38.9 | 38.1 | 37.4 | 36.9 | | | | |
| Analysis by | Glycated | 28.0 | 27.0 | 26.8 | 26.4 | 26.1 | | | | |
| CEX-UPLC | Region 2 | 54.3 | 55.8 | 56.6 | 57.8 | 58.1 | | | | |
| (%) | Region 3 | 5.8 | 5.3 | 5.3 | 4.9 | 5.1 | | | | |
| Particulate | ≥10 μm | 25 | 18 | 62 | NR | 49 | | | | |
| Matter by Light | ≥25 μm | 2 | 0 | 0 | NR | 0 | | | | |
| Obscuration | | | | | | | | | | |
| (#/container) | | | | | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY5, not more intensely colored than Reference Solution BY5;
Not > III, not more turbid than Reference Suspension III;
NR, not required;
Ph. Eur., European Pharmacopeia;
SE, size-exclusion;
UPLC, ultra performance liquid chromatography;
USP; United States Pharmacopeia

TABLE 34
Stability of 5.0 mg/ml mAb1 Liquid Drug Product Stored at 25° C./60% RH and 40° C./75% RH

| Formulation | | mAb1 drug product; sterile, vialed mAb1 recombinant protein, 5.0 mg/mL, in an aqueous | | | | | | |
|---|---|---|---|---|---|---|---|---|
| | | buffered solution, pH 5.0, containing 30 mM acetate, 10% (w/v) sucrose, and 0.2% (w/v) | | | | | | |
| | | polysorbate 20 | | | | | | |
| Container Closure | | 6R (5 mL) USP/Ph. Eur. Type 1 borosilicate glass vial; 20 mm FluroTec ®-coated | | | | | | |
| | | chlorobutyl stopper; 20 mm Flip-Off ® seal | | | | | | |
| | | | 25° C./60% RH Storage | | | 40° C./75% RH Storage | | |
| | | | (months) | | | (months) | | |
| Assay | | t = 0 | 1 | 3 | 6 | 0.5 | 1 | 3 |
| Physical Form/Condition | | LEFVP | LEFVP | LEFVP | LEFVP | LEFVP | LEFVP | LEFVP |
| Clarity | | Not > I | Not > II | Not > I | Not > II | Not > II | Not > II | Not > II |
| Color | | Not > BY7 | Not > BY7 | Not > BY7 | Not > BY7 | Not > BY7 | Not > BY7 | Not > BY7 |
| pH | | 5.0 | 5.0 | 5.0 | 5.0 | 5.0 | 5.0 | 5.0 |
| Total Protein (mg/mL) | | 5.2 | 5.2 | 5.2 | 5.2 | 5.2 | 5.2 | 5.4 |
| Polysorbate 20 (%) | | 0.21 | NR | NR | 0.21 | NR | NR | 0.20 |
| Reduced | Purity | 97.5 | 97.1 | 96.9 | 96.6 | 96.7 | 96.6 | 94.5 |
| MCE (%) | LMW | 0.2 | 0.4 | 0.5 | 0.6 | 0.5 | 0.4 | 1.5 |
| Non-reduced | Purity | 97.4 | 97.0 | 97.1 | 96.9 | 97.0 | 96.1 | 92.9 |
| MCE (%) | LMW | 2.2 | 2.6 | 2.4 | 2.6 | 2.6 | 3.5 | 5.8 |
| SE-UPLC | MP Purity | 98.5 | 98.7 | 98.4 | 98.3 | 98.6 | 97.5 | 95.4 |
| (%) | LMW | 0.0 | 0.0 | 0.2 | 0.0 | 0.0 | 0.8 | 0.7 |
| | HMW | 1.5 | 1.3 | 1.5 | 1.7 | 1.4 | 1.7 | 3.9 |
| Charge Variant | Region 1 | 41.0 | 31.5 | 24.6 | 19.7 | 17.8 | 14.2 | 26.2 |
| Analysis by | Glycated | 28.6 | 22.6 | 14.7 | 10.1 | 9.4 | 6.2 | 13.2 |
| CEX-UPLC | Region 2 | 53.9 | 64.4 | 71.9 | 76.9 | 78.4 | 81.5 | 67.7 |
| (%) | Region 3 | 5.2 | 4.1 | 3.5 | 3.4 | 3.7 | 4.3 | 6.1 |
| Particulate | ≥10 μm | 37 | NR | 451 | 580 | NR | NR | 484 |
| Matter by Light | ≥25 μm | 0 | NR | 8 | 10 | NR | NR | 5 |
| Obscuration | | | | | | | | |
| (#/container) | | | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY7, not more intensely colored than Reference Solution BY7;
Not > I, not more turbid than Reference Suspension I;
Not > II, not more turbid than Reference Suspension II;
NR, not required;
Ph. Eur., European Pharmacopeia;
RH, relative humidity;
SE, size-exclusion;
UPLC, ultra performance liquid chromatography;
USP, United States Pharmacopeia

TABLE 35
Stability of 50.0 mg/ml mAb1 Liquid Drug Product Stored at 25° C./60% RH and 40° C./75% RH

| Formulation | | mAb1 drug product; sterile, vialed mAb1 recombinant protein, 50.0 mg/mL, in an aqueous | | | | | | |
|---|---|---|---|---|---|---|---|---|
| | | buffered solution, pH 5.0, containing 30 mM acetate, 10% (w/v) sucrose, and 0.2% (w/v) | | | | | | |
| | | polysorbate 20 | | | | | | |
| Container Closure | | 6R (5 mL) USP/Ph. Eur. Type 1 borosilicate glass vial; 20 mm FluroTec ®-coated | | | | | | |
| | | chlorobutyl stopper; 20 mm Flip-Off ® seal | | | | | | |
| | | | 25° C./60% RH Storage | | | 40° C./75% RH Storage | | |
| | | | (months) | | | (months) | | |
| Assay | | t = 0 | 1 | 3 | 6 | 0.5 | 1 | 3 |
| Physical Form/Condition | | LEFVP | LEFVP | LEFVP | LEFVP | LEFVP | LEFVP | LEFVP |
| Clarity | | Not > III | Not > III | Not > III | Not > III | Not > III | Not > III | Not > III |
| Color | | Not > BY5 | Not > BY5 | Not > BY5 | Not > BY5 | Not > BY5 | Not > BY5 | Not > BY4 |
| pH | | 5.0 | 5.0 | 5.0 | 5.0 | 5.0 | 5.0 | 5.0 |
| Total Protein (mg/mL) | | 51.0 | 48.6 | 50.3 | 51.0 | 49.5 | 49.3 | 51.1 |
| Polysorbate 20 (%) | | 0.19 | NR | NR | 0.20 | NR | NR | 0.19 |
| Reduced | Purity | 97.1 | 97.4 | 96.7 | 96.5 | 96.7 | 95.9 | 93.5 |
| MCE (%) | LMW | 0.3 | 0.2 | 0.6 | 0.3 | 0.4 | 1.0 | 2.6 |
| Non-reduced | Purity | 96.9 | 96.2 | 96.4 | 93.2 | 95.9 | 94.7 | 91.6 |
| MCE (%) | LMW | 2.3 | 2.9 | 2.5 | 3.0 | 2.9 | 3.8 | 6.0 |
| SE-UPLC | MP Purity | 97.9 | 97.8 | 97.5 | 97.4 | 97.0 | 96.1 | 90.4 |
| (%) | LMW | 0.0 | 0.0 | 0.1 | 0.0 | 0.4 | 0.5 | 0.4 |
| | HMW | 2.1 | 2.2 | 2.5 | 2.6 | 2.6 | 3.5 | 9.2 |
| Charge Variant | Region 1 | 39.9 | 33.3 | 25.4 | 20.9 | 18.5 | 17.4 | 28.6 |
| Analysis by | Glycated | 28.0 | 23.2 | 15.6 | 11.4 | 11.1 | 8.8 | 13.1 |
| CEX-UPLC | Region 2 | 54.3 | 62.0 | 69.6 | 74.7 | 77.2 | 77.1 | 59.4 |
| (%) | Region 3 | 5.8 | 4.7 | 5.1 | 4.4 | 4.3 | 5.5 | 12.0 |
| Particulate | ≥10 μm | 25 | NR | 55 | 75 | NR | NR | 20 |
| Matter by Light | ≥25 μm | 2 | NR | 1 | 1 | NR | NR | 1 |
| Obscuration | | | | | | | | |
| (#/container) | | | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY4, not more intensely colored than Reference Solution BY4;
Not > BY5, not more intensely colored than Reference Solution BY5;
Not > III, not more turbid than Reference Suspension III;
NR, not required;
Ph. Eur., European Pharmacopeia;
RH, relative humidity;
SE, size-exclusion;
UPLC, ultra performance liquid chromatography;
USP, United States Pharmacopeia

TABLE 36
Stability of 5.0 mg/nl mAb1 Liquid Drug Product Effect of Agitation and Freeze/Thaw

| | |
|---|---|
| Formulation | mAb1 drug product; sterile, vialed mAb1 recombinant protein, 5.0 mg/mL, in an aqueous |
| | buffered solution, pH 5.0, containing 30 mM acetate, 10% (w/v) sucrose, and 0.2% (w/v) |
| | polysorbate 20 |
| Container/Closure | 6R (5 mL) USP/Ph. Eur. Type 1 borosilicate glass vial; 20 mm FluroTec ®-coated |
| | chlorobutyl stopper; 20 mm Flip-Off ® seal |

| | | | Agitation | | Freezing/Thawing | |
|---|---|---|---|---|---|---|
| | | | (minutes) | | (cycles) | |
| Assay | | T = 0 | 60 | 120 | 2 | 4 |
| Physical Form/Condition | | LEFVP | LEFVP | LEFVP | LEFVP | LEFVP |
| Clarity | | Not > I | Not > I | Not > I | Not > I | Not > I |
| Color | | Not > BY7 | Not > BY7 | Not > BY7 | Not > BY7 | Not > BY7 |
| pH | | 5.0 | 5.0 | 5.0 | 5.0 | 5.0 |
| Total Protein (mg/mL) | | 5.2 | 5.2 | 5.2 | 5.2 | 5.2 |
| Polysorbate 20 (%) | | 0.21 | NR | 0.20 | NR | 0.21 |
| Reduced | Purity | 97.5 | NR | 97.2 | NR | 97.3 |
| MCE (%) | LMW | 0.2 | NR | 0.2 | NR | 0.2 |
| Non-reduced | Purity | 97.4 | NR | 97.2 | NR | 97.8 |
| MCE (%) | LMW | 2.2 | NR | 2.4 | NR | 1.9 |
| SE-UPLC | MP Purity | 98.5 | 98.3 | 98.3 | 98.4 | 98.5 |
| (%) | LMW | 0.0 | 0.0 | 0.0 | 0.0 | 0.0 |
| | HMW | 1.5 | 1.7 | 1.7 | 1.7 | 1.6 |
| Charge Variant | Region 1 | 41.0 | 40.9 | 40.8 | 39.2 | 39.5 |
| Analysis by | Glycated | 28.6 | 28.5 | 28.5 | 28.1 | 28.1 |
| CEX-UPLC | Region 2 | 53.9 | 53.9 | 54.2 | 55.7 | 55.3 |
| (%) | Region 3 | 5.2 | 5.2 | 5.1 | 5.1 | 5.2 |
| Particulate | ≥10 μm | 37 | NR | 125 | NR | 294 |
| Matter by Light | ≥25 μm | 0 | NR | 0 | NR | 15 |
| Obscuration | | | | | | |
| (#/container) | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY7, not more intensely colored than Reference Solution BY7;
Not > I, not more turbid than Reference Suspension I;
NR, not required;
Ph. Eur., European Pharmacopeia;
SE, size-exclusion;
UPLC, ultra performance liquid chromatography;
USP, United States Pharmacopeia

TABLE 37
Stability of 50.0 mg/nl mAb1 Liquid Drug Product Effect of Agitation and Freeze/Thaw

| | |
|---|---|
| Formulation | mAb1 drug product; sterile, vialed mAb1 recombinant protein, 50.0 mg/mL, in an aqueous |
| | buffered solution, pH 5.0, containing 30 mM acetate, 10% (w/v) sucrose, and 0.2% (w/v) |
| | polysorbate 20 |
| Container/Closure | 6R (5 mL) USP/Ph. Eur. Type 1 borosilicate glass vial; 20 mm FluroTec ®-coated |
| | chlorobutyl stopper; 20 mm Flip-Off ® seal |

| | | | Agitation | | Freezing/Thawing | |
|---|---|---|---|---|---|---|
| | | | (minutes) | | (cycles) | |
| Assay | | T = 0 | 60 | 120 | 2 | 4 |
| Physical Form/Condition | | LEFVP | LEFVP | LEFVP | LEFVP | LEFVP |
| Clarity | | Not > III | Not > III | Not > III | Not > II | Not > III |
| Color | | Not > BY5 | Not > BY5 | Not > BY5 | Not > BY5 | Not > BY5 |
| pH | | 5.0 | 5.0 | 5.0 | 5.0 | 5.0 |
| Total Protein (mg/mL) | | 51.0 | 49.4 | 49.1 | 49.4 | 48.1 |
| Polysorbate 20 (%) | | 0.19 | NR | 0.19 | NR | 0.19 |
| Reduced | Purity | 97.1 | NR | 97.1 | NR | 97.3 |
| MCE (%) | LMW | 0.3 | NR | 0.2 | NR | 0.1 |
| Non-Reduced | Purity | 96.9 | NR | 96.6 | NR | 96.8 |
| MCE (%) | LMW | 2.3 | NR | 2.8 | NR | 2.4 |
| SE-UPLC | MP Purity | 97.9 | 97.8 | 97.9 | 97.8 | 97.8 |
| (%) | LMW | 0.0 | 0.0 | 0.0 | 0.0 | 0.0 |
| | HMW | 2.1 | 2.2 | 2.2 | 2.2 | 2.2 |
| Charge Variant | Region 1 | 39.9 | 40.0 | 40.1 | 39.0 | 39.0 |
| Analysis by | Glycated | 28.0 | 27.9 | 28.1 | 28.0 | 28.1 |
| CEX-UPLC | Region 2 | 54.3 | 54.2 | 54.1 | 55.6 | 55.6 |
| (%) | Region 3 | 5.8 | 5.8 | 5.8 | 5.5 | 5.4 |
| Particulate | ≥10 μm | 25 | NR | 12 | NR | 34 |
| Matter by Light | ≥25 μm | 2 | NR | 0 | NR | 1 |
| Obscuration | | | | | | |
| (#/container) | | | | | | |

CEX, cation exchange;
HMW, high molecular weight;
LEFVP, liquid essentially free from visible particulates;
LMW, low molecular weight;
MCE, microchip capillary electrophoresis;
MP, main peak;
Not > BY5, not more intensely colored than Reference Solution 5;
Not > II, not more turbid than Reference Suspension II;
Not > III, not more turbid than Reference Suspension III;
NR, not required;
Ph. Eur., European Pharmacopeia;
SE, size-exclusion;
UPLC, ultra performance liquid chromatography;
USP, United States Pharmacopeia

### Example 3: Lyophilization Cycle Development

The lyophilization process that was developed for clinical production consists of: freezing, primary drying, and secondary drying. The lyophilization process was developed using an FTS LyoStar™ III lyophilizer based on a partial cake collapse temperature of −31.2° C., determined for the frozen formulated drug substance using a freeze-dry microscope. During primary drying, the product temperature did not exceed the partial cake collapse temperature, thereby maintaining cake integrity during the lyophilization cycle. The secondary drying process was developed to ensure the drug product has low residual moisture content.

The lyophilization cycle takes approximately 63 hours to produce freeze-dried mAb1 drug product in 5 mL Type 1 glass vials containing 2.5 mL of 2 mg/mL mAb1 formulated drug substance. The lyophilization cycle contains the steps shown in Table 38, below.

TABLE 38
Lyophilization Cycle

| | Shelf Temperature | Shelf | Holding | |
|---|---|---|---|---|
| Lyophilization | Ramp Rate | Temperature | Time | |
| Step | (Ramp Duration) | (° C.) | (hours) | Chamber Pressure |
| Loading | NA | 5-25 | NA | Ambient Pressure |
| Holding | 0.5° C./minute | 5 | 1 | Ambient Pressure |
| Freezing | 0.5° C./minute (100 minutes) | −45 | 2 | Ambient Pressure |
| Primary Drying | 0.5° C./minute (50 minutes) | −20 | 40 | 100 mTorr |
| Secondary Drying | 0.2° C./minute (300 minutes) | 40 | 10 | 100 mTorr |
| Temperature Ramp | 0.5° C./minute (30 minutes) | 25 | 1 | 100 mTorr |
| for Stoppering | | | | |
| Back Fill with | NA | 25 | NA | 80% of Atmospheric |
| Gas Nitrogen | | | | Pressure (608,000 |
| | | | | mTorr) |
| Stoppering | NA | 25 | NA | 80% of Atmospheric |
| | | | | Pressure (608,000 |
| | | | | mTorr) |

NA, not applicable

### Example 4: Development of Stable Liquid Anti-MUC16×Anti-CD3 Bispecific Antibody Formulations for Intravenous Administration, and Stability of Formulations

Development of intravenous (IV) formulations, including formulations comprising 5 mg/ml of mAb1 and 50 mg/ml of mAb1 was undertaken to identify a pH to minimize the change of glycation at HCDR3-Lys98 in the MUC16-binding arm, an appropriate buffer and concentration to maintain the pH and overcome the observed Donnan effect during manufacturing, a suitable thermal stabilizer at a concentration to maintain desired viscosity and tonicity, and a suitable surfactant at a concentration sufficient for dilution during IV administration, all while maintaining a stable liquid formulation.

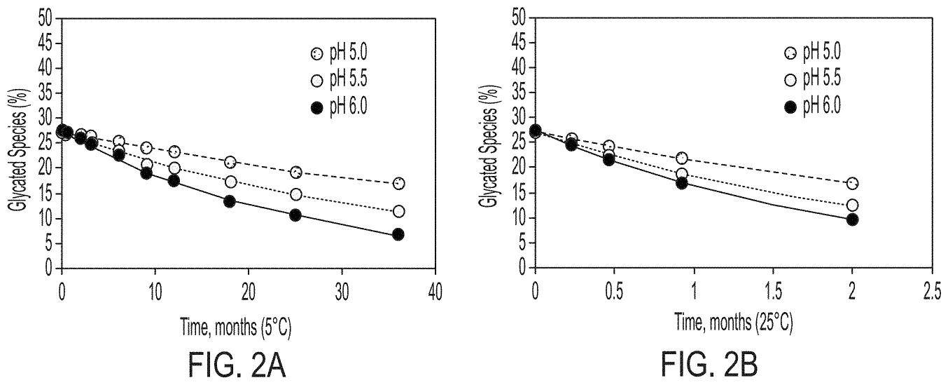

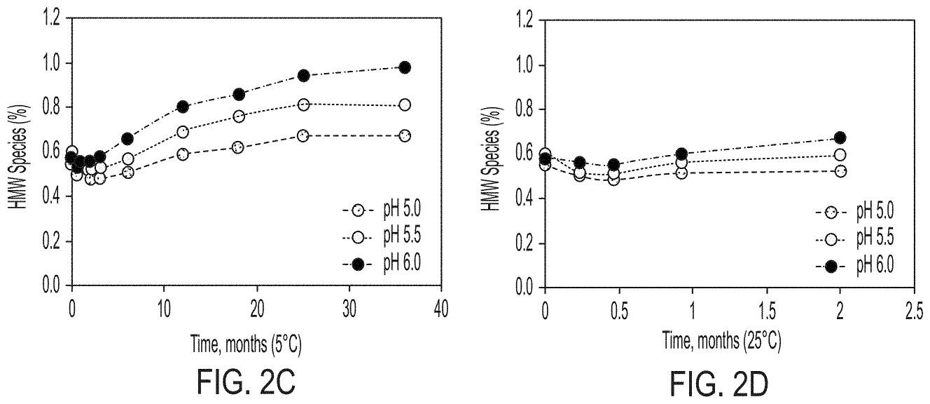

As discussed above, glycation at HC-CDR3-Lys98 of the MUC16 binding arm has a direct impact on potency. Deglycation leads to an increase in potency as measured by bioassay. The main formulation factor that impacts the rate of deglycation in mAb1 is pH. The effect of pH on the levels of glycation and high-molecular-weight (HMW) species was investigated in liquid formulations by incubating 2 mg/mL mAb1 at 5° C. for 36 months or 25° C. for 2 months in 10 mM histidine, 10% (w/v) sucrose, and 0.05% (w/v) polysorbate 20 at three different pH's: 5.0, 5.5, and 6.0 (FIGS. 2A-2D. The major degradation pathways observed under these conditions are the formation of HMW species and the deglycation at HC-CDR3-Lys98 as detected by CEX-UPLC. As the pH of mAb1 formulation was reduced, the levels of HMW species formed and the rates of deglycation decreased.

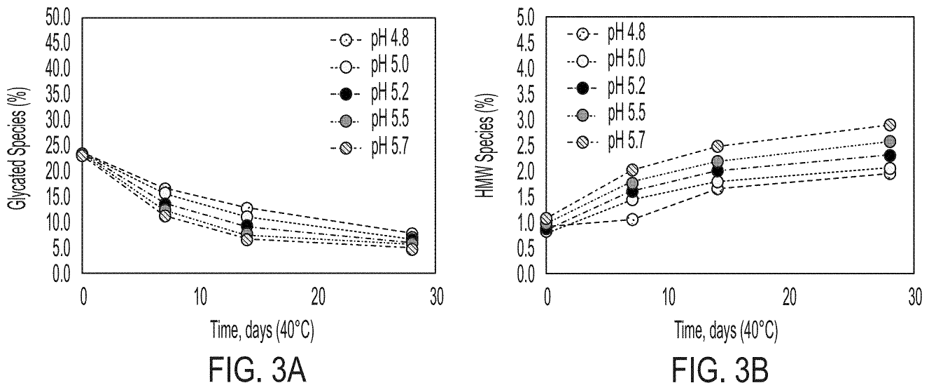

Since histidine is not a good buffer at pH 5.0, sodium acetate was chosen as the buffer for the mAb1 liquid formulations. The effect of pH was investigated by incubating 50 mg/mL mAb1 in 10 mM acetate with 5% (w/v) sucrose and five different pH's: 4.8, 5.0, 5.2, 5.5, and 5.7 under thermal stress. After incubating at 40° C. for 28 days, the major degradation pathways were the formation of HMW species and the deglycation at HC-CDR3-Lys98. Consistent with the observation in histidine buffer, the levels of HMW species formed and the rates of deglycation decreased at lower pH (FIGS. 3A-3B). pH 5.0 was selected as the target to produce a robust formulation for which normal manufacturing variations will remain in the range required to stabilize mAb1.

The effect of acetate concentration on the stability of 150 mg/mL mAb1 formulations at pH 5.0 was examined in liquid formulations. Formulations containing acetate buffer ranging from 21 to 40 mM were incubated at 45° C. for 14 days. The higher temperature allows the rapid detection of protein degradation. The analyses revealed that formation of HMW species and charge variants were the main degradation pathways. An increase in acetate concentration led to increased HMW species and 30 mM was selected based on the data. A concentration of 30 mM acetate is able to address the Donnan effect observed during the manufacturing process. As a result of the Donnan effect, 30 mM acetate is required to maintain the formulation pH at 5.0. This acetate concentration provided improved stabilization with respect to the formation of HMW species under this stress condition as compared to formulations with higher concentrations of acetate (Table 39). Therefore, 30 mM was selected as the acetate concentration for the mAb1 liquid formulations for IV administration.

TABLE 39
Stability of 150 mg/mL mAb1 in Acetate Buffer, pH 5.0, Incubated at 45° C. for 14 Days

| Formulation | 150 mg/mL mAb1 in acetate buffer, pH 5.0 | | | | |
|---|---|---|---|---|---|
| Fill Volume | 0.5 mL | | | | |
| Container/Closure | 2R USP/Ph. Eur. Type 1 borosilicate glass vial; 13 mm FluroTec ®-coated | | | | |
| | butyl elastomeric stopper; 13 mm Flip-Off ® seal | | | | |
| Storage Condition | 45° C. for 14 days | | | | |
| Buffer | | t = 0^{a} | 21 mM acetate | 30 mM acetate | 40 mM acetate |
| Color and Appearance^{b} | | Pass | Pass | Pass | Pass |
| Turbidity (Increase in OD at 405 nm)^{c} | | 0.00 | 0.02 | 0.03 | 0.06 |
| pH | | 5.0 | 5.0 | 5.1 | 5.1 |
| Total Protein by RP-UPLC (mg/mL) | | 155 | 152 | 166 | 161 |
| SE-UPLC | HMW | 1.3 | 18.1 | 20.0 | 21.9 |
| (%) | Purity | 98.4 | 80.1 | 78.7 | 76.8 |
| | LMW | 0.3 | 1.8 | 1.4 | 1.3 |
| CEX-UPLC | Region 1 | 47.1 | 22.3 | 23.7 | 24.0 |
| (%) | Glycated | 28.9 | 5.0 | 5.5 | 5.5 |
| | Region 2 | 48.4 | 73.3 | 72.0 | 71.6 |
| | Region 3 | 4.5 | 4.4 | 4.4 | 4.4 |

^{a}t = 0 results reported represent an average of the starting values for all samples in this study
^{b}Sample passes color and visual appearance assessment if it is clear to slightly opalescent, essentially free from visible particulates, and colorless to pale yellow.
^{c}Compared to t = 0 for each formulation
CEX, cation exchange;
DS, drug substance;
HMW, high molecular weight;
LMW, low molecular weight;
OD, optical density;
Ph. Eur; European Pharmacopeia;
SE, size-exclusion;
UPLC, ultra performance liquid chromatography;
USP, United States Pharmacopeia

The effect of sucrose on the stability of mAb1 was examined in liquid formulations (150 mg/mL mAb1, 30 mM acetate, pH 5.0 with 0 to 13% [w/v] sucrose) under freeze/thaw and thermal stresses. The higher temperature allows the rapid detection of protein degradation under these stresses. Under freeze/thaw stress, formation of HMW species was the main degradation pathway (Table 40). Under 45° C. thermal stress, formation of HMW and LMW species and change of charge variants were the main degradation pathways. Formulations with a higher concentration of sucrose (10%) provided improved stabilization by reducing the level of HMW and LMW species and the rate of deglycation at HC-CDR3-Lys98 (Table 41). To maintain desired viscosity and tonicity for mAb1, 10% (w/v) sucrose was selected as the thermal stabilizer for the liquid formulations for IV administration.

TABLE 40
Stability of 150 mg/mL mAb1 in 30 mM Acetate Buffer with Different Concentrations
of Sucrose, pH 5.0, Under Freeze/Thaw Stress Condition

| | |
|---|---|
| Formulation | 150 mg/mL mAb1 in 30 mM acetate buffer, pH 5.0 |
| Fill Volume | 0.5 mL |
| Container/Closure | 2R USP/Ph. Eur. Type 1 borosilicate glass vial; 13 mm FluroTec ®-coated |
| | butyl rubber stopper; 13 mm Flip-Off ® seal |
| Stress Condition | 8 freeze/thaw cycles |

| | | | 0% (w/v) | 2% (w/v) | 5% (w/v) | 10% (w/v) | 13% (w/v) |
|---|---|---|---|---|---|---|---|
| Thermal Stabilizer | | t = 0^{a} | sucrose | sucrose | sucrose | sucrose | sucrose |
| Color and Appearance^{b} | | Pass | Pass | Pass | Pass | Pass | Pass |
| Turbidity (Increase in OD at 405 nm)^{c} | | 0.00 | 0.02 | 0.00 | 0.00 | 0.00 | 0.01 |
| pH | | 5.0 | 5.1 | 5.1 | 5.1 | 5.1 | 5.0 |
| Total Protein (mg/mL) | | 155 | 148 | 150 | 162 | 149 | 157 |
| SE-UPLC | HMW | 1.3 | 2.4 | 1.5 | 1.4 | 1.4 | 1.4 |
| (%) | Purity | 98.4 | 97.1 | 98.2 | 98.3 | 98.3 | 98.4 |
| | LMW | 0.3 | 0.5 | 0.3 | 0.3 | 0.3 | 0.3 |
| CEX-UPLC | Region 1 | 47.1 | 46.3 | 46.8 | 46.8 | 47.0 | 46.9 |
| (%) | Glycated | 28.9 | 28.7 | 28.3 | 28.4 | 27.9 | 28.5 |
| | Region 2 | 48.4 | 48.5 | 48.4 | 48.4 | 48.3 | 48.4 |
| | Region 3 | 4.5 | 5.2 | 4.8 | 4.8 | 4.8 | 4.7 |

^{a}t = 0 results reported represent an average of the starting values for all samples in this study
^{b}Sample passes color and visual appearance assessment if it is clear to slightly opalescent, essentially free from visible particulates, and colorless to pale yellow.
^{c}Compared to t = 0 for each formulation
CEX, cation exchange;
DS, drug substance;
HMW, high molecular weight;
LMW, low molecular weight;
OD, optical density;
Ph. Eur; European Pharmacopeia;
SE, size-exclusion;
UPLC, ultra performance liquid chromatography;
USP, United States Pharmacopeia

TABLE 41
Stability of 150 mg/mL mAb1 in 30 mM Acetate Buffer with Different
Concentrations of Sucrose, pH 5.0, Incubated at 45° C. for 14 Days

| | |
|---|---|
| Formulation | 150 mg/mL mAb1 in 30 mM acetate buffer, pH 5.0 |
| Fill Volume | 0.5 mL |
| Container/Closure | 2R USP/Ph. Eur. Type 1 borosilicate glass vial; 13 mm FluroTec ®-coated |
| | butyl rubber stopper; 13 mm Flip-Off ® seal |
| Storage Condition | 45° C. for 14 days |

| | | | 0% (w/v) | 2% (w/v) | 5% (w/v) | 10% (w/v) | 13% (w/v) |
|---|---|---|---|---|---|---|---|
| Thermal stabilizer | | t = 0^{a} | sucrose | sucrose | sucrose | sucrose | sucrose |
| Color and Appearance^{b} | | Pass | Pass | Pass | Pass | Pass | Pass |
| Turbidity (Increase in OD at 405 nm)^{c} | | 0.00 | 0.03 | 0.03 | 0.03 | 0.01 | 0.02 |
| pH | | 5.0 | 5.1 | 5.0 | 5.0 | 5.0 | 5.0 |
| Total Protein (mg/mL) | | 155 | 166 | 156 | 157 | 161 | 165 |
| SE-UPLC | HMW | 1.3 | 20.0 | 26.0 | 23.4 | 17.3 | 14.5 |
| (%) | Purity | 98.4 | 78.7 | 72.8 | 75.6 | 81.8 | 84.7 |
| | LMW | 0.3 | 1.4 | 1.2 | 1.1 | 0.9 | 0.8 |
| CEX-UPLC | Region 1 | 47.1 | 23.7 | 24.7 | 25.1 | 27.1 | 28.3 |
| (%) | Glycated | 28.9 | 5.5 | 6.1 | 5.1 | 6.7 | 8.1 |
| | Region 2 | 48.4 | 72.0 | 70.8 | 70.4 | 68.3 | 67.1 |
| | Region 3 | 4.5 | 4.4 | 4.5 | 4.5 | 4.6 | 4.5 |

^{a}t = 0 results reported represent an average of the starting values for all samples in this study
^{b}Sample passes color and visual appearance assessment if it is clear to slightly opalescent, essentially free from visible particulates, and colorless to pale yellow.
^{c}Compared to t = 0 for each formulation
CEX, cation exchange;
DS, drug substance;
HMW, high molecular weight;
LMW, low molecular weight;
OD, optical density;
Ph. Eur; European Pharmacopeia;
SE, size-exclusion;
UPLC, ultra performance liquid chromatography;
USP, United States Pharmacopeia

The need for a surfactant was demonstrated during initial development of the mAb1 formulations. When no surfactant was present, an increase in mAb1 HMW species was observed when the formulation was agitated by vortexing. Addition of surfactant stabilized mAb1 to agitation stress. In the initial development, polysorbate 20 was chosen as the surfactant because of the improved thermal stability compared to polysorbate 80. The mAb1 formulation intended for IV administration also requires polysorbate 20 in the formulation to stabilize mAb1 when diluted in 0.9% sodium chloride for IV administration.

The effect of polysorbate 20 on the stability of mAb1 was examined in liquid formulations (50 mg/mL mAb1, 30 mM acetate, 10% [w/v] sucrose, pH 5.0 with 0 to 0.25% [w/v] polysorbate 20) under agitation and thermal stresses.

Under agitation stress, mAb1 was stable in all liquid formulations tested (Table 42). Under 45° C. thermal stress, formation of HMW species and charge variants were the main degradation pathways (Table 43). Increasing polysorbate 20 concentration had no meaningful impact to the formation of charge variant, however, some increases in the levels of HMW species formed were observed with increasing polysorbate 20 concentration. Since these mAb1 formulations are intended for IV delivery by diluting in 0.9% sodium chloride, the formulations will contain 0.2% (w/v) polysorbate 20. 0.2% polysorbate 20 stabilizes mAb1 and provides sufficient stabilization of mAb1 when diluted for IV administration.

TABLE 42
Stability of 50 mg/mL mAb1 in 30 mM Acetate Buffer, 10% (w/v)
Sucrose, pH 5.0 with Polysorbate 20, Agitated by Vortexing

| | |
|---|---|
| Formulation | 50 mg/mL mAb1 in 30 mM acetate buffer, 10% (w/v) sucrose, pH 5.0 |
| Fill Volume | 0.5 mL |
| Container/Closure | 2R USP/Ph. Eur. Type 1 borosilicate glass vial; 13 mm FluroTec ®-coated |
| | butyl rubber stopper; 13 mm Flip-Off ® seal |
| Stress Condition | Vortexed at 1,000 rpm for 60 min |

| | | | 0% (w/v) | 0.05% (w/v) | 0.10% (w/v) | 0.20% (w/v) | 0.25% (w/v) |
|---|---|---|---|---|---|---|---|
| Surfactant | | t = 0^{a} | polysorbate 20 | polysorbate 20 | polysorbate 20 | polysorbate 20 | polysorbate 20 |
| Color and Appearance^{b} | | Pass | Pass | Pass | Pass | Pass | Pass |
| Turbidity (Increase in OD at 405 nm)^{c} | | 0.00 | 0.00 | 0.01 | 0.01 | 0.00 | 0.00 |
| pH | | 5.1 | 5.0 | 5.0 | 5.1 | 5.0 | 5.1 |
| Total Protein (mg/mL) | | 51 | 48 | 49 | 49 | 49 | 49 |
| SE-UPLC | HMW | 1.7 | 1.7 | 1.7 | 1.7 | 1.7 | 1.7 |
| (%) | Purity | 98.0 | 98.0 | 98.0 | 98.0 | 98.0 | 98.0 |
| | LMW | 0.3 | 0.3 | 0.3 | 0.3 | 0.3 | 0.3 |
| CEX-UPLC | Region 1 | 43.4 | 43.3 | 43.3 | 43.5 | 43.3 | 43.6 |
| (%) | Glycated | 30.9 | 31.0 | 31.0 | 30.9 | 30.9 | 31.3 |
| | Region 2 | 51.8 | 51.9 | 51.9 | 51.6 | 51.9 | 51.8 |
| | Region 3 | 4.8 | 4.8 | 4.8 | 4.9 | 4.8 | 4.6 |

^{a}t = 0 results reported represent an average of the starting values for all samples in this study
^{b}Sample passes color and visual appearance assessment if it is clear to slightly opalescent, essentially free from visible particulates, and colorless to pale yellow.
^{c}Compared to t = 0 for each formulation
CEX, cation exchange;
DS, drug substance;
HMW, high molecular weight;
LMW, low molecular weight;
OD, optical density;
Ph. Eur; European Pharmacopeia;
SE, size-exclusion;
UPLC, ultra performance liquid chromatography;
USP, United States Pharmacopeia

TABLE 43
Stability of 50 mg/mL mAb1 in 30 mM Acetate Buffer, 10% (w/v) Sucrose,
pH 5.0 with Polysorbate 20, Incubated at 45° C. for 14 Days

| | |
|---|---|
| Formulation | 50 mg/mL mAb1 in 30 mM acetate buffer, 10% (w/v) sucrose, pH 5.0 |
| Fill Volume | 0.5 mL |
| Container/Closure | 2R USP/Ph. Eur. Type 1 borosilicate glass vial; 13 mm FluroTec ®-coated |
| | butyl rubber stopper; 13 mm Flip-Off ® seal |
| Storage Condition | 45° C. for 14 days |

| | | | 0% (w/v) | 0.05% (w/v) | 0.10% (w/v) | 0.20% (w/v) | 0.25% (w/v) |
|---|---|---|---|---|---|---|---|
| Surfactant | | t = 0^{a} | polysorbate 20 | polysorbate 20 | polysorbate 20 | polysorbate 20 | polysorbate 20 |
| Color and Appearance^{b} | | Pass | Pass | Pass | Pass | Pass | Pass |
| Turbidity (Increase in OD at 405 nm)^{c} | | 0.00 | 0.01 | 0.01 | 0.02 | 0.01 | 0.01 |
| pH | | 5.1 | 5.1 | 5.1 | 5.1 | 5.1 | 5.1 |
| Total Protein (mg/mL) | | 51 | 50 | 50 | 51 | 51 | 50 |
| SE-UPLC | HMW | 1.7 | 4.2 | 5.1 | 5.1 | 5.8 | 6.0 |
| (%) | Purity | 98.0 | 95.0 | 94.0 | 94.0 | 93.3 | 93.1 |
| | LMW | 0.3 | 0.9 | 0.9 | 0.9 | 0.9 | 0.9 |
| CEX-UPLC | Region 1 | 43.4 | 17.7 | 17.4 | 17.4 | 16.6 | 16.8 |
| (%) | Glycated | 30.9 | 8.0 | 7.8 | 7.9 | 7.3 | 7.4 |
| | Region 2 | 51.8 | 75.1 | 74.5 | 74.7 | 74.8 | 74.5 |
| | Region 3 | 4.8 | 7.2 | 8.1 | 7.9 | 8.6 | 8.7 |

^{a}t = 0 results reported represent an average of the starting values for all samples in this study
^{b}Sample passes color and visual appearance assessment if it is clear to slightly opalescent, essentially free from visible particulates, and colorless to pale yellow.
^{c}Compared to t = 0 for each formulation
CEX, cation exchange;
DS, drug substance;
HMW, high molecular weight;
LMW, low molecular weight;
OD, optical density;
Ph. Eur; European Pharmacopeia;
SE, size-exclusion;
UPLC, ultra performance liquid chromatography;
USP, United States Pharmacopeia

Long-term, accelerated, and stress stability studies were performed to verify the stability of the liquid formulations. The results indicated that these formulations provide adequate stability.

### Example 5: Development of Stable Liquid Anti-MUC16×Anti-CD3 Bispecific Antibody Formulations for Subcutaneous Administration, and Stability of Formulations

Development of subcutaneous (SC) formulations, including formulations comprising 150 mg/ml of mAb1 was undertaken to identify the excipients and concentrations maximized for SC administration and stability.

The buffer, pH and surfactant parameters were identified for the IV formulation (Example 4) and these parameters were applied to the subcutaneous formulation. Surfactant (polysorbate 20) concentration was also characterized during IV formulation development and the data was used to select a surfactant concentration suitable for a subcutaneous formulation. The buffer, pH and surfactant type and concentration were held constant at the following levels during the subcutaneous formulation development for mAb1:
pH: 5.0. To minimize the rate of deglycation and formation of HMW species
Buffer: 30 mM sodium acetate. To maintain pH 5.0, even with the Donnan effect observed during processing
Surfactant: 0.05% (w/v) polysorbate 20. To balance stability to agitation stress while minimizing the impact to thermal stability

Other factors that were evaluated for the mAb1 SC formulation are:
mAb1 concentration: assessed in the range 50-150 mg/mL
   mAb1 concentration to achieve the required clinical dose while maintaining stability for at least 24 months when stored at 2-8° C. and maintaining a viscosity less than 20 cP at 20° C., with a target osmolality of 290-400 mOsm/kg.
Sucrose concentration: assessed in the range 2-10%
   Sucrose concentration to provide sufficient thermal stability, while minimizing impacts to osmolality and viscosity
   At least 2% (w/v) sucrose is required to stabilize mAb1 to freeze/thaw stress
Arginine concentration: assessed in the range 0-100 mM
   Assess ability of arginine to reduce viscosity and assess the impact to stability and osmolality

Formulations evaluated for SC administration are shown in Table 44, below.

TABLE 44
Formulations tested for SC Administration of mAb1

| Formulation | [Protein] | [Sucrose] | [Arginine] | Acetate | Polysorbate |
|---|---|---|---|---|---|
| number | (mg/mL) | (%, w/v) | (mM) | (mM) | 20 (%) |
| 1 | 100 | 2 | 37.5 | 30 | 0.05 |
| 2 | 100 | 10 | 0 | 30 | 0.05 |
| 3 | 50 | 2 | 100 | 30 | 0.05 |
| 4 | 150 | 10 | 100 | 30 | 0.05 |
| 5 | 50 | 2 | 0 | 30 | 0.05 |
| 6 | 50 | 10 | 100 | 30 | 0.05 |
| 7 | 150 | 10 | 50 | 30 | 0.05 |
| 8 | 50 | 6 | 50 | 30 | 0.05 |
| 9 | 100 | 6 | 100 | 30 | 0.05 |
| 10 | 150 | 4.44 | 0 | 30 | 0.05 |
| 11 | 100 | 6 | 50 | 30 | 0.05 |
| 12 | 150 | 2 | 100 | 30 | 0.05 |

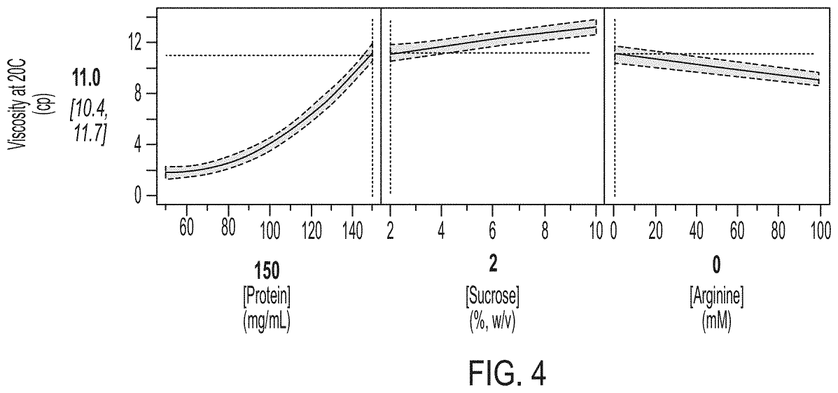

Viscosity Analysis—FIG. 4 shows the dependence of mAb1 viscosity as a function of mAb1 concentration, sucrose concentration, and arginine concentration. The following observations were made from this study: (i) The main factor contributing to viscosity is mAb1 concentration. Over the range of 50-150 mg/mL, the viscosity increases exponentially from about 2 cP to about 11 cP (at 20° C.); (ii) There is a small dependency of viscosity on sucrose. At 150 mg/mL mAb1 concentration, when sucrose is varied from 2-10% (w/v), the viscosity increases from about 11 cP to 13 cP (at 20° C.); and (iii) The effect of arginine concentration on viscosity is similar in magnitude to sucrose, but increasing arginine decreases viscosity from about 11 cP to 9 cP (at 20° C.).

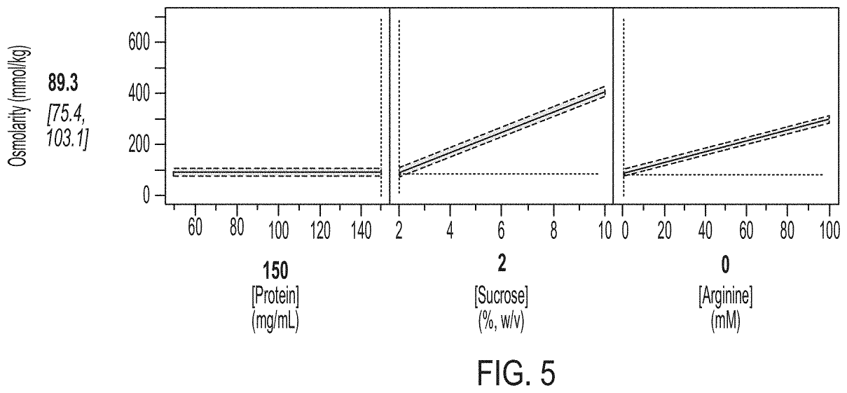

Osmolality Analysis—FIG. 5 shows the dependence of mAb1 osmolality as a function of mAb1 concentration, sucrose concentration, and arginine concentration. The following observations were made from this study: (i) mAb1 concentration makes a negligible contribution to osmolality over the range of 50-150 mg/mL; (ii) The main factor contributing to osmolality is sucrose. At 150 mg/mL mAb1 concentration, when sucrose is varied from 2-10% (w/v), the osmolality increases from about 90 mOsm/kg to about 410 mOsm/kg; and (iii) Increasing arginine concentration also results in an increase in osmolality. At 150 mg/mL mAb1, increasing arginine from 0-100 mM increases osmolality from about 90 mOsm/kg to about 300 mOsm/kg.

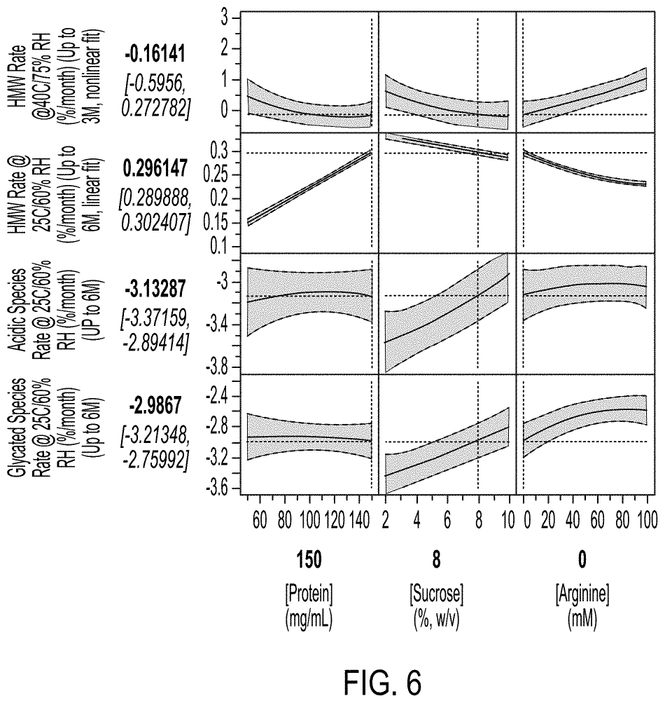

Stability Analysis—FIG. 6 shows the stability of mAb1 as a function of mAb1 concentration, sucrose concentration, and arginine concentration. In this study, HMW species formation at 25° C./60% RH and 40° C./75% RH was assessed. Additionally, loss of acidic species and the loss of glycated species was assessed under the same conditions. The following observations were made: (i) Increasing sucrose concentration led to decreased rates of HMW formation at both 25° C./60% RH and 40° C./75% RH; (ii) Increasing arginine concentration led to a decreased rate of HMW formation at 25° C./60% RH but an increased rate of HMW formation at 40° C./75% RH; (iii) Increasing sucrose concentration led to a reduced rate of acidic species and glycated species formation at 25° C./60% RH. (Acidic and glycated species decrease over time, so a less negative rate means less degradation over time); and (iv) Increasing arginine concentration led to a reduced rate of glycated species formation at 25° C./60% RH.

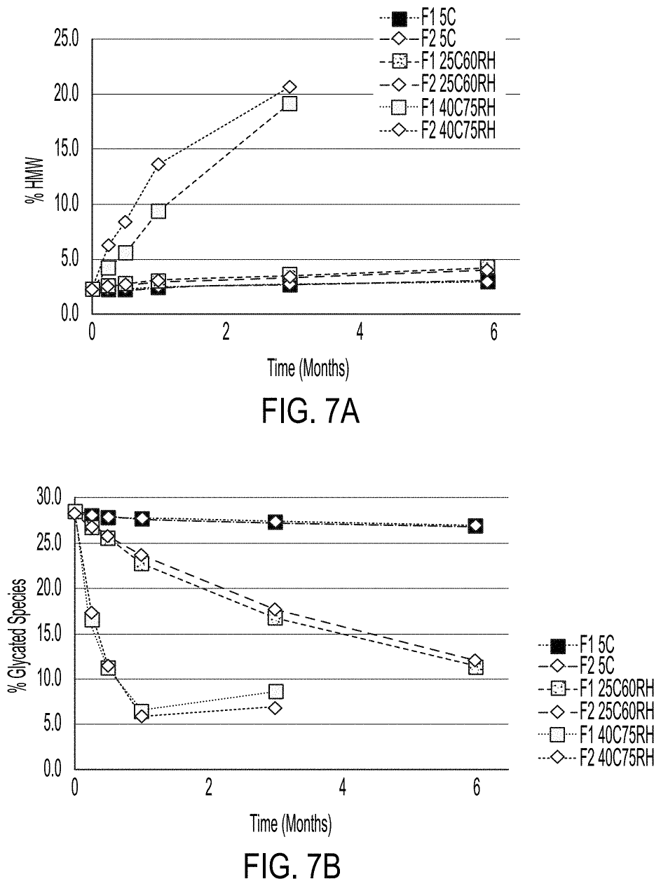

Based on the prior development studies (e.g., Examples 1 and 2) and this study, two lead formulations were selected for further stability, viscosity, and osmolality assessment. The two lead formulations and the respective viscosities and osmolalities are shown in Table 45, below. Both formulations met the viscosity and osmolality targets at an mAb1 concentration of 150 mg/mL. Both formulations showed comparable stability when incubated for 3 months at 40° C./75% RH, 6 months at 25° C./60% RH or 6 months at 2-8° C. (FIGS. 7A and 7B). Both formulations showed comparable changes in HMW species and glycated species.

TABLE 45
Exemplary mAb1 SC Formulations

| | VISCOSITY | OSMO- |
|---|---|---|
| | AT 20 C. | LALITY |
| FORMULATIONS | (CP) | (MMOL/KG) |
| 150 mg/mL mAb1, 30 mM acetate, 8% | 12.6 | 333 |
| (w/v) sucrose, 0.05% (w/v) PS20, pH 5.0 | | |
| 150 mg/mL mAb1, 30 mM acetate, 7% | 11.6 | 389 |
| (w/v) sucrose, 50 mM arginine, 0.05% | | |
| (w/v) PS20, pH 5.0 | | |

Based on these data, both formulations are comparable. Arginine was not selected as an excipient in the subcutaneous formulation as it provides little improvement to stability or viscosity but results in an increase in osmolality. Thus, a preferred exemplary subcutaneous formulation is: 150 mg/mL mAb1; 30 mM acetate, pH 5.0; 8% w/v sucrose; and 0.05% w/v polysorbate 20.

Stability studies were also initiated to evaluate the storage, stress, and accelerated stability of the mAb1 formulations. The stability studies included a subcutaneous (SC) formulation (150 mg/mL mAb1 in 30 mM sodium acetate, 8% (w/v) sucrose, 0.05% (w/v) polysorbate 20, pH 5.0) stored in Schott 6R borosilicate glass vials. The liquid formulations were incubated under storage, stress, and accelerated conditions. The stress and accelerated conditions were selected to simulate the conditions beyond which the drug product will be subjected during manufacturing and handling, and to elucidate the degradation pathways for mAb1. Stability under additional stresses including agitation and freeze/thaw was also evaluated.

The antibody (mAb1) in the evaluated formulations was physically and chemically stable when stored at 5° C. for at least 6 months (Table 46). No appreciable changes in stability were detected in any of the monitored attributes at 5° C. in 6R vials. Results from the analysis of the mAb1 formulations after incubation under accelerated and stress conditions are provided in Table 47. Following incubation for 6 months at 25° C./60% RH, an increase of 1.7% HMW species was observed by SE-UPLC. Different trends for charge variant were observed by CEX-UPLC and iCIEF due to different sensitivities of each assay. An increase in Region 1, with a concomitant decrease in Region 2, was observed by iCIEF. A decrease in Region 1, with a concomitant increase in Region 2, was observed by CEX-UPLC when stored at 25° C./60% RH for up to 6 months because of the de-glycation at HC-CDR3-Lys98 of MUC16 arm (the de-glycation also occurs at the thermal stress condition). The results from the accelerated condition indicated that the liquid formulations are stable under thermal stress in 6R vials.

Following incubation for 3 months at 40° C./75% RH, increases of 15.9 and 1.1% in HMW and LMW species, respectively, were observed by SE-UPLC. Different trends for charge variant were observed by CEX-UPLC and iCIEF due to different sensitivities of each assay. An increase in Region 1, with concomitant decreases in Regions 2 and 3, was observed by iCIEF after incubation at 40° C./75% RH for up to 3 months, likely due to the deamidation on asparagine or glutamine. After incubation for up to 2 months at 40° C./75% RH, there is a decrease in Region 1, with a concomitant increase in Region 2 observed by CEX-UPLC because of the de-glycation at HC-CDR3-Lys98 of MUC16 arm (the de-glycation also occurs at the accelerated stress condition). This is followed by an apparent increase in Region 1, with a concomitant decrease in Region 2, observed by CEX-UPLC at the 3 month time point, likely due to the competing deamidation reaction. There is also a 16.3% increase in Region 3 after 3 months at 40° C./75% RH that is observed by CEX-UPLC. This increase was determined to be comprised of oligomeric species of mAb1, including mainly tetramer, pentamer, hexamer and heptamer species. Results from incubation of the mAb1 formulations at 40° C./75% RH indicated that formation of HMW and LMW species and the change in distribution of charge variants are the major degradation pathways for the mAb1 drug product.

The mAb1 formulations were physically and chemically stable when vortexed for either 60 or 120 minutes (Table 48). No appreciable change in the physical or chemical stability was detected in any of the monitored attributes. The mAb1 formulations were also physically and chemically stable when subjected to 4 freezing and thawing cycles (Table 48). No appreciable change in the physical or chemical stability was detected in any of the monitored attributes.

TABLE 46
Stability of mAb1 Drug Product Stored at 5° C.

| | |
|---|---|
| Formulation | 150.0 mg/mL REGN4018, 30 mM acetate, 8% (w/v) sucrose, and 0.05% (w/v) |
| | polysorbate 20. |
| Storage Container | USP/EP Type 1 borosilicate 6R ISO glass vial; 20 mm West S10-F451 4432/50 |
| | B2-40 liquid stopper; West 20 mm aluminum seal with flip-off button |
| Storage Condition | 5° C., Upright |

| | Quality | | | | |
|---|---|---|---|---|---|
| | Acceptance | Length of Storage (months) | | | |
| Assay | Criteria ^{a} | T = 0 | 1 | 3 | 6 |
| Physical | Liquid | LEFVP | LEFVP | LEFVP | LEFVP |
| Form/ | essentially | | | | |
| Condition | free from | | | | |
| | visible | | | | |
| | particulates | | | | |
| Clarity | Not more | Not > II | Not > II | Not > II | Not > II |
| | turbid than | | | | |
| | reference | | | | |
| | suspension IV | | | | |
| Color | Not more | Not > BY4 | Not > BY4 | Not > BY4 | Not > BY4 |
| | intensely | | | | |
| | colored than | | | | |
| | reference | | | | |
| | solution BY2 | | | | |
| pH | 4.7 to 5.3 | 5.0 | 5.0 | 5.0 | 5.0 |
| Total Protein | 135 to 165 | 144.8 | 145.3 | 150.7 | 146.2 |
| (SoloVPE) | mg/mL | | | | |
| Potency by | 50-150% | 103 | NR | NR | 146 |
| Bioassay | | | | | |
| Purity by | a. Purity ≥90% | 97.8 | 97.7 | 97.5 | 97.4 |
| SE-UPLC | total peak area | | | | |
| | b. ≤5% LMW | 0.0 | 0.0 | 0.0 | 0.0 |
| | species | | | | |
| | c. ≤7% HMW | 2.2 | 2.3 | 2.5 | 2.6 |
| | species | | | | |
| Charge | Report % | 39.9 | NR | NR | 40.2 |
| Variant | Region 1 | | | | |
| Analysis | Report % | 41.9 | NR | NR | 42.7 |
| by iCIEF | Region 2 | | | | |
| | Report % | 18.2 | NR | NR | 17.1 |
| | Region 3 | | | | |
| Charge | a. 15-60% | 45.7 | 45.5 | 44.3 | 43.5 |
| Variant | Region 1 | | | | |
| Analysis | b. % Glycated | 32.4 | 32.6 | 31.6 | 31.2 |
| by CEX | species | | | | |
| | (report %) | | | | |
| | c. ≥35% | 48.3 | 48.8 | 49.9 | 50.9 |
| | Region 2 | | | | |
| | d. ≤25% | 6.0 | 5.8 | 5.8 | 5.6 |
| | Region 3 | | | | |
| Particulate | ≥10 | 13 | NR | NR | 22 |
| Matter (light | μm: ≤6000 | | | | |
| obscuration) | (particles/ | | | | |
| | container) | | | | |
| | ≥25 | 0 | NR | NR | 4 |
| | μm: ≤600 | | | | |
| | (particles/ | | | | |
| | container) | | | | |
| Particulate | Report | 3641 | NR | NR | 1829 |
| Matter | particle/mL: | | | | |
| (MFI ™) | 2 μm ≤ | | | | |
| | x < 10 μm | | | | |
| | (particle/mL) | | | | |
| | ≥10 μm | 240 | NR | NR | 86 |
| | (particle/mL) | | | | |
| | ≥25 μm | 34 | NR | NR | 23 |
| | (particle/mL) | | | | |
| Polysorbate | 0.05 ± 0.025% | 0.05 | NR | NR | 0.06 |
| 20 Content | PS20 | | | | |

^{a} Criteria adopted from FBP-015-FD FDG platform Quality Target with program specific adjustment.
NR indicates tests not being performed at set time point.
CEX, cation exchange; DS, drug substance; FDG, formulation development group; HMW, high molecular weight; iCIEF, imaged capillary isoelectric focusing; LMW, low molecular weight; MFI, Micro-Flow Imaging ™; MP, main peak; SE-UPLC, size-exclusion ultra high-performance liquid chromatography; NR, not required; LEFVP, liquid essential free of visible particles.

TABLE 47
Stability of mAb1 Drug Product Incubated at 25° C./60% RH and 40° C.

| | |
|---|---|
| Formulation | 150.0 mg/mL REGN4018, 30 mM acetate, 8% (w/v) sucrose, and 0.05% (w/v) |
| | polysorbate 20. |
| Storage Container | USP/EP Type 1 borosilicate 6R ISO glass vial; 20 mm West S10-F451 4432/50 |
| | B2-40 liquid stopper; West 20 mm aluminum seal with flip-off button |

| | | Storage Condition | | | | | | | | |
|---|---|---|---|---|---|---|---|---|---|---|
| | | No | 25° C./60% RH Storage | | | | 40° C. Storage | | | |
| | | Storage | (months) | | | | (months) | | | |
| Assay | | T = 0 | 0.5 | 1 | 3 | 6 | 0.25 | 0.5 | 1 | 3 |
| Physical | Liquid | LEFVP | LEFVP | LEFVP | LEFVP | LEFVP | LEFVP | LEFVP | LEFVP | LEFVP |
| Form/ | essentially | | | | | | | | | |
| Condition | free from | | | | | | | | | |
| | visible | | | | | | | | | |
| | particulates | | | | | | | | | |
| Clarity | Not more | Not > II | Not > III | Not > III | Not > III | Not > III | Not > II | Not > III | Not > III | Not > III |
| | turbid than | | | | | | | | | |
| | reference | | | | | | | | | |
| | suspension IV | | | | | | | | | |
| Color | Not more | Not > | Not > | Not > | Not > | Not > | Not > | Not > | Not > | Not > |
| | intensely | BY4 | BY4 | BY4 | BY4 | BY4 | BY4 | BY4 | BY4 | BY3 |
| | colored than | | | | | | | | | |
| | reference | | | | | | | | | |
| | solution BY2 | | | | | | | | | |
| pH | 4.7 to 5.3 | 5.0 | 5.0 | 5.0 | 5.0 | 5.0 | 5.0 | 5.0 | 5.0 | 5.0 |
| Total Protein | 135 to 165 | 144.8 | 149.0 | 145.3 | 153.9 | 148.4 | 145.2 | 148.9 | 144.8 | 147.9 |
| (SoloVPE) | mg/mL | | | | | | | | | |
| Potency by | 50-150% | 103 | NR | NR | NR | 148 | NR | NR | NR | 132 |
| Bioassay | | | | | | | | | | |
| Purity by | a. Purity ≥90% | 97.8 | 97.4 | 97.2 | 96.6 | 96.0 | 95.7 | 94.8 | 92.2 | 80.9 |
| SE-UPLC | total peak area | | | | | | | | | |
| | b. ≤5% LMW | 0.0 | 0.0 | 0.0 | 0.0 | 0.1 | 0.3 | 0.0 | 0.1 | 1.1 |
| | species | | | | | | | | | |
| | c. ≤7% HMW | 2.2 | 2.6 | 2.8 | 3.3 | 3.9 | 4.0 | 5.2 | 7.7 | 18.1 |
| | species | | | | | | | | | |
| Charge | Report % | 39.9 | NR | NR | NR | 49.0 | NR | NR | NR | 72.0 |
| Variant | Region 1 | | | | | | | | | |
| Analysis | Report % | 41.9 | NR | NR | NR | 33.5 | NR | NR | NR | 18.0 |
| by iCIEF | Region 2 | | | | | | | | | |
| | Report % | 18.2 | NR | NR | NR | 17.5 | NR | NR | NR | 10.0 |
| | Region 3 | | | | | | | | | |
| Charge | a. 15-60% | 45.7 | 41.3 | 38.8 | 29.2 | 23.1 | 30.8 | 23.4 | 18.3 | 25.1 |
| Variant | Region 1 | | | | | | | | | |
| Analysis | b. % Glycated | 32.4 | 29.5 | 27.5 | 18.6 | 13.2 | 20.3 | 13.5 | 7.5 | 12.2 |
| by CEX | species | | | | | | | | | |
| | (report %) | | | | | | | | | |
| | c. ≥35% | 48.3 | 53.0 | 56.2 | 65.6 | 71.3 | 63.7 | 69.5 | 72.4 | 55.6 |
| | Region 2 | | | | | | | | | |
| | d. ≤25% | 6.0 | 5.6 | 5.0 | 5.2 | 5.6 | 5.5 | 7.1 | 9.3 | 19.3 |
| | Region 3 | | | | | | | | | |
| Particulate | ≥10 | 13 | NR | NR | NR | 25 | NR | NR | NR | 48 |
| Matter (light | μm: ≤6000 | | | | | | | | | |
| obscuration) | particles/ | | | | | | | | | |
| | container | | | | | | | | | |
| | ≥25 | 0 | NR | NR | NR | 2 | NR | NR | NR | 5 |
| | μm: ≤600 | | | | | | | | | |
| | particles/ | | | | | | | | | |
| | container | | | | | | | | | |
| Particulate | 2 μm ≤ | 3641 | NR | NR | NR | 1238 | NR | NR | NR | 653 |
| Matter | x < 10 μm | | | | | | | | | |
| (MFI ™) | (particles/mL) | | | | | | | | | |
| | ≥10 μm | 240 | NR | NR | NR | 29 | NR | NR | NR | 33 |
| | (particles/mL) | | | | | | | | | |
| | ≥25 μm | 34 | NR | NR | NR | 2 | NR | NR | NR | 2 |
| | (particles/mL) | | | | | | | | | |
| Polysorbate | Report % | 0.05 | NR | NR | NR | 0.06 | NR | NR | NR | 0.05 |
| 20 Content | PS20 | | | | | | | | | |

NR indicates tests not being performed at set time point.
CEX, cation exchange;
DS, drug substance;
FDG, formulation development group;
HMW, high molecular weight;
iCIEF, imaged capillary isoelectric focusing;
LMW, low molecular weight;
MFI, Micro-Flow Imaging ™;
MP, main peak;
SE-UPLC, size-exclusion ultra high-performance liquid chromatography;
NR, not required

TABLE 48
Stability of mAb1 Drug Product - Effect of Agitation and Freeze/Thaw

| | |
|---|---|
| Formulation | 150.0 mg/mL REGN4018, 30 mM acetate, 8% (w/v) sucrose, and 0.05% (w/v) |
| | polysorbate 20. |
| Storage Container | USP/EP Type 1 borosilicate 6R ISO glass vial; 20 mm West S10-F451 4432/50 |
| | B2-40 liquid stopper; West 20 mm aluminum seal with flip-off button |

| | | Storage Condition | | | | |
|---|---|---|---|---|---|---|
| | | | Agitation | | Freeze/Thaw | |
| | | No Stress | (minutes) | | (cycles) | |
| Assay | | T = 0 | 60 | 120 | 2 | 4 |
| Physical | Liquid | LEFVP | LEFVP | LEFVP | LEFVP | LEFVP |
| Form/ | essentially | | | | | |
| Condition | free from | | | | | |
| | visible | | | | | |
| | particulates | | | | | |
| Clarity | Not more | Not > II | Not > II | Not > II | Not > II | Not > II |
| | turbid than | | | | | |
| | reference | | | | | |
| | suspension IV | | | | | |
| Color | Not more | Not > BY4 | Not > BY4 | Not > BY4 | Not > BY4 | Not > BY4 |
| | intensely | | | | | |
| | colored than | | | | | |
| | reference | | | | | |
| | solution BY2 | | | | | |
| pH | 4.7 to 5.3 | 5.0 | 5.0 | 5.0 | 5.0 | 5.0 |
| Total Protein | 135 to 165 | 144.8 | 144.4 | 144.4 | 145.7 | 147.9 |
| (SoloVPE) | mg/mL | | | | | |
| Potency by | 50-150% | 103 | NR | 138 | NR | 97 |
| Bioassay | | | | | | |
| Purity by | a. Purity ≥90% | 97.8 | 97.8 | 97.8 | 97.8 | 97.8 |
| SE-UPLC | total peak area | | | | | |
| | b. ≤5% LMW | 0.0 | 0.0 | 0.0 | 0.0 | 0.1 |
| | species | | | | | |
| | c. ≤7% HMW | 2.2 | 2.2 | 2.2 | 2.2 | 2.1 |
| | species | | | | | |
| Charge | Report % | 39.9 | NR | 40.3 | NR | 39.5 |
| Variant | Region 1 | | | | | |
| Analysis | Report % | 41.9 | NR | 42.3 | NR | 42.5 |
| by iCIEF | Region 2 | | | | | |
| | Report % | 18.2 | NR | 17.5 | NR | 18.0 |
| | Region 3 | | | | | |
| Charge | a. 15-60% | 45.7 | 45.7 | 45.8 | 45.2 | 45.5 |
| Variant | Region 1 | | | | | |
| Analysis | b. % Glycated | 32.4 | 32.4 | 32.5 | 32.0 | 32.3 |
| by CEX | species | | | | | |
| | (report %) | | | | | |
| | c. ≥35% | 48.3 | 48.3 | 48.3 | 48.8 | 48.4 |
| | Region 2 | | | | | |
| | d. ≤25% | 6.0 | 6.0 | 6.0 | 6.1 | 6.0 |
| | Region 3 | | | | | |
| Particulate | ≥10 | 13 | NR | 4 | NR | 40 |
| Matter (light | μm: ≤6000 | | | | | |
| obscuration) | particles/ | | | | | |
| | container | | | | | |
| | ≥25 | 0 | NR | 0 | NR | 3 |
| | μm: ≤600 | | | | | |
| | particles/ | | | | | |
| | container | | | | | |
| Particulate | 2 μm ≤ | 3641 | NR | 1470 | NR | 271 |
| Matter | x < 10 μm | | | | | |
| (MFI ™) | (particles/mL) | | | | | |
| | ≥10 μm | 240 | NR | 73 | NR | 13 |
| | (particles/mL) | | | | | |
| | ≥25 μm | 34 | NR | 0 | NR | 2 |
| | (particles/mL) | | | | | |
| Polysorbate | Report % | 0.05 | NR | 0.05 | NR | 0.05 |
| 20 Content | PS20 | | | | | |

NR indicates tests not being performed at set time point.
DS, drug substance;
FDG, formulation development group;
HMW, high molecular weight;
LMW, low molecular weight;
MFI, Micro-Flow Imaging ™;
MP, main peak;
SE-UPLC, size-exclusion ultra high-performance liquid chromatography;
NR, not required

Additional stability studies were initiated to determine the long-term storage, accelerated stability (temperatures above storage conditions), and stress stability (40° C./75% RH, agitation, freezing and thawing) of mAb1 formulations at 150 mg/ml antibody. mAb1 formulations with 150 mg/mL antibody were filled in 5 mL polycarbonate vials for the agitation, freeze/thaw, frozen storage, and accelerated and stress storage conditions. The polycarbonate vials are representative of the storage container used for the mAb1 formulations (formulated drug substance) that are manufactured in the GMP facility. The tested formulations contained 150 mg/mL purified mAb1 in an aqueous buffered solution containing 30 mM sodium acetate, pH 5.0, 8% (w/v) sucrose and 0.05% (w/v) PS20.

No appreciable changes in the physical or chemical stability of the mAb1 formulations were detected when stored at −80° C. and −30° C. for up to 6 months (Table 49 and Table 50). These results indicate that mAb1 (150 mg/mL) is stable for at least 6 months when stored frozen at storage conditions.

Results from the research accelerated stability studies are presented in Tables 51A and 51B. No appreciable changes in the monitored attributes were observed after incubating the mAb1 formulations (150 mg/mL antibody) at −20° C. for up to 6 months. An increase in protein concentration was observed by SoloVPE after incubation at 25° C./60% RH for 6 months, likely due to sample evaporation. An increase in HMW species was observed by SE-UPLC after incubation at 5° C. and 25° C./60% RH for 6 months. A decrease in Region 1 (acidic species) with a concomitant increase in Region 2 (main peak) were observed by CEX-UPLC after incubation at 25° C./60% RH for 6 months because of the de-glycation at HC-CDR3-Lys98 of MUC16 arm. An increase in Region 1 (acidic species) with a concomitant decrease in Region 2 (main peak) were observed by iCIEF after incubation at 25° C./60% RH for 6 months likely due to deamidation. These results indicate that mAb1 (150 mg/mL) formulations can withstand incubation at −20° C. for at least 6 months and at 5° C. for 3 months without compromising either the physical or chemical stability of the protein. The mAb1 formulations can also withstand short exposures to temperatures of 25° C./60% RH.

Results from the research stress stability studies are presented in Tables 51A and 51B, and Table 52. The mAb1 (150 mg/mL) formulations were physically and chemically stable when agitated (vortexed) for up to 120 minutes or subjected to up to four cycles of freezing and thawing. An increase in protein concentration was observed by SoloVPE after incubation at 40° C./75% RH for up to 3 months, likely due to sample evaporation. Increases in HMW and LMW species were observed by SE-UPLC after incubation at 40° C./75% RH for up to 3 months. A decrease in Region 1 with a concomitant increase in Region 2 were observed by CEX-UPLC after incubation at 40° C./75% RH because of the de-glycation at HC-CDR3-Lys98 of MUC16 arm. After 2 months of incubation at 40° C./75% RH, there is a decrease in Region 1 and concomitant increase in Region 2. However, after the three-month time point the trend reverses and there is an apparent increase in Region 1 and Region 3 with concomitant decrease in Region 2. The increase in Region 1 is likely due to competing deamidation on asparagine or glutamine, whereas the increase in Region 3 was determined to be comprised of oligomeric species of mAb1, including mainly tetramer, pentamer, hexamer and heptamer species. The results from the accelerated and stressed conditions indicated that HMW, LMW, and charge variants are the major degradation pathways for mAb1 (150 mg/mL).

TABLE 49
Stability of mAb1 Formulated Drug Substance at −80° C.

| | |
|---|---|
| Formulation | 150 mg/mL REGN4018, 30 mM acetate, pH 5.0, 8% (w/v) sucrose, 0.05% |
| | polysorbate 20 |
| Fill Volume | 2.5 mL |
| Storage Container | 5 mL Cellon polycarbonate vial with HDPE lined closure |
| Storage Condition | −80° C., Upright vial orientation |

| | Quality | | | | |
|---|---|---|---|---|---|
| | Acceptance | Length of Storage (months) | | | |
| Assay | Criteria^{a} | T = 0 | 1 | 3 | 6 |
| Physical | Liquid | LEFVP | LEFVP | LEFVP | LEFVP |
| Form/ | essentially | | | | |
| Condition | free from | | | | |
| | visible | | | | |
| | particulates | | | | |
| Clarity | Not more | Not > II | Not > II | Not > II | Not > II |
| | turbid than | | | | |
| | reference | | | | |
| | suspension IV | | | | |
| Color | Not more | Not > BY4 | Not > BY4 | Not > BY4 | Not > BY4 |
| | intensely | | | | |
| | colored than | | | | |
| | reference | | | | |
| | solution BY2 | | | | |
| pH | 4.8 to 5.2 | 5.0 | 5.0 | 5.0 | 5.0 |
| Total | 45 to 55 | 146.3 | 148.9 | 153.2 | 148.7 |
| Protein | mg/mL | | | | |
| Purity by | a. Purity ≥90% | 97.8 | 97.9 | 97.8 | 97.9 |
| SE-UPLC | total peak area | | | | |
| | b. ≤5% LMW | 0.0 | 0.0 | 0.0 | 0.0 |
| | species | | | | |
| | c. ≤7% HMW | 2.2 | 2.1 | 2.2 | 2.1 |
| | species | | | | |
| Charge | Report % | 39.4 | | | 41.7 |
| Variant | Region 1 | | | | |
| Analysis | Report % | 42.5 | | | 41.6 |
| by iCIEF | Region 2 | | | | |
| | Report % | 18.1 | | | 16.7 |
| | Region 3 | | | | |
| Charge | a. 15-60% | 45.8 | 45.9 | 45.1 | 45.5 |
| Variant | Region 1 | | | | |
| Analysis | b. % Glycated | 32.5 | 32.6 | 32.0 | 32.2 |
| by CEX | species | | | | |
| | (report %) | | | | |
| | c. ≥35% | 48.2 | 48.5 | 48.6 | 48.6 |
| | Region 2 | | | | |
| | d. ≤25% | 6.0 | 5.6 | 6.3 | 5.9 |
| | Region 3 | | | | |
| Particulate | 2 μm ≤ | 9537 | | | 250 |
| Matter | x < 10 μm | | | | |
| (MFI ™) | (particle/mL) | | | | |
| | ≥10 μm | 622 | | | 4 |
| | (particle/mL) | | | | |
| | ≥25 μm | 76 | | | 0 |
| | (particle/mL) | | | | |
| Polysorbate | Report % | 0.05 | | | |
| 20 Content | PS20 | | | | |

^{a}Criteria adopted from FBP-015-FD FDG platform Quality Target with program specific adjustment. Results from the assays without Quality Target reported percentage for information only. Boxes greyed to indicate tests not being performed at set time point.
CEX, cation exchange; DS, drug substance; FDG, formulation development group; HMW, high molecular weight; iCIEF, imaged capillary isoelectric focusing; LEFVP, liquid essential free of visible particles; LMW, low molecular weight; MFI, Micro-Flow Imaging ™; MP, main peak; SE-UPLC, size-exclusion ultra high-performance liquid chromatography.

TABLE 50
Stability of mAb1 Formulated Drug Substance at −30° C.

| | |
|---|---|
| Formulation | 150 mg/mL REGN4018, 30 mM acetate, pH 5.0, 8% (w/v) sucrose, 0.05% |
| | polysorbate 20 |
| Fill Volume | 2.5 mL |
| Storage Container | 5 mL Cellon polycarbonate vial with HDPE lined closure |
| Storage Condition | −30° C., Upright vial orientation |

| | Quality | | | | |
|---|---|---|---|---|---|
| | Acceptance | Length of Storage (months) | | | |
| Assay | Criteria^{a} | T = 0 | 1 | 3 | 6 |
| Physical | Liquid | LEFVP | LEFVP | LEFVP | LEFVP |
| Form/ | essentially | | | | |
| Condition | free from | | | | |
| | visible | | | | |
| | particulates | | | | |
| Clarity | Not more | Not > II | Not > II | Not > II | Not > II |
| | turbid than | | | | |
| | reference | | | | |
| | suspension IV | | | | |
| Color | Not more | Not > BY4 | Not > BY4 | Not > BY4 | Not > BY4 |
| | intensely | | | | |
| | colored than | | | | |
| | reference | | | | |
| | solution BY2 | | | | |
| pH | 4.8 to 5.2 | 5.0 | 5.0 | 5.0 | 5.0 |
| Total | 45 to 55 | 146.3 | 146.1 | 148.3 | 149.2 |
| Protein | mg/mL | | | | |
| Purity by | a. Purity ≥90% | 97.8 | 97.9 | 97.8 | 97.9 |
| SE-UPLC | total peak area | | | | |
| | b. ≤5% LMW | 0.0 | 0.0 | 0.0 | 0.0 |
| | species | | | | |
| | c. ≤7% HMW | 2.2 | 2.1 | 2.2 | 2.1 |
| | species | | | | |
| Charge | Report % | 39.4 | | | 41.6 |
| Variant | Region 1 | | | | |
| Analysis | Report % | 42.5 | | | 42.4 |
| by iCIEF | Region 2 | | | | |
| | Report % | 18.1 | | | 16.1 |
| | Region 3 | | | | |
| Charge | a. 15-60% | 45.8 | 46.0 | 45.3 | 45.7 |
| Variant | Region 1 | | | | |
| Analysis | b. % Glycated | 32.5 | 32.3 | 32.2 | 32.5 |
| by CEX | species | | | | |
| | (report %) | | | | |
| | c. ≥35% | 48.2 | 48.3 | 48.5 | 48.5 |
| | Region 2 | | | | |
| | d. ≤25% | 6.0 | 5.7 | 6.2 | 5.8 |
| | Region 3 | | | | |
| Particulate | 2 μm ≤ | 9537 | | | 469 |
| Matter | x < 10 μm | | | | |
| (MFI ™) | (particle/mL) | | | | |
| | ≥10 μm | 622 | | | 13 |
| | (particle/mL) | | | | |
| | ≥25 μm | 76 | | | 4 |
| | (particle/mL) | | | | |
| Polysorbate | Report % | 0.05 | | | |
| 20 Content | PS20 | | | | |
| Potency by | 50% to 150% | 94 | | | 149 |
| Bioassay | of reference | | | | |
| | standard | | | | |

^{a}Criteria adopted from FBP-015-FD FDG platform Quality Target with program specific adjustment. Results from the assays without Quality Target reported percentage for information only. Boxes greyed to indicate tests not being performed at set time point.
CEX, cation exchange; DS, drug substance; FDG, formulation development group; HMW, high molecular weight; iCIEF, imaged capillary isoelectric focusing; LEFVP, liquid essential free of visible particles; LMW, low molecular weight; MFI, Micro-Flow Imaging ™; MP, main peak; SE-UPLC, size-exclusion ultra high-performance liquid chromatography.

TABLE 51A
Stability of mAb1 Formulated Drug Substance Incubated at −20° C., 5° C.

| Formulation | | | 150 mg/mL REGN4018, 30 mM acetate, pH 5.0, 8% (w/v) sucrose, 0.05% | | | | | | |
|---|---|---|---|---|---|---|---|---|---|
| | | | polysorbate 20 | | | | | | |
| Fill Volume | | | 2.5 mL | | | | | | |
| Storage Container | | | 5 mL Cellon polycarbonate vial with HDPE lined closure | | | | | | |
| | | Storage Condition | | | | | | | |
| | Quality | No | −20° C. Storage | | | 5° C. Storage | | | |
| | Acceptance | Storage | (months) | | | (months) | | | |
| Assay | Criteria^{a} | T = 0 | 1 | 3 | 6 | 0.5 | 1 | 3 | 6 |
| Physical | Liquid | LEFVP | LEFVP | LEFVP | LEFVP | LEFVP | LEFVP | LEFVP | LEFVP |
| Form/ | essentially | | | | | | | | |
| Condition | free from | | | | | | | | |
| | visible | | | | | | | | |
| | particulates | | | | | | | | |
| Clarity | Not more | Not > II | Not > II | Not > II | Not > II | Not > II | Not > II | Not > II | Not > II |
| | turbid than | | | | | | | | |
| | reference | | | | | | | | |
| | suspension IV | | | | | | | | |
| Color | Not more | Not > BY4 | Not > BY4 | Not > BY4 | Not > BY4 | Not > BY4 | Not > BY4 | Not > BY4 | Not > BY4 |
| | intensely | | | | | | | | |
| | colored than | | | | | | | | |
| | reference | | | | | | | | |
| | solution BY2 | | | | | | | | |
| pH | 4.8 to 5.2 | 5.0 | 5.0 | 5.0 | 5.0 | 5.0 | 5.0 | 5.0 | 5.0 |
| Total | 45 to 55 | 146.3 | 146.4 | 150.4 | 154.2 | 146.8 | 151.1 | 150.9 | 161.3 |
| Protein | mg/mL | | | | | | | | |
| Purity by | a. Purity ≥90% | 97.8 | 97.8 | 97.8 | 97.9 | 97.7 | 97.7 | 97.5 | 97.4 |
| SE-UPLC | total peak area | | | | | | | | |
| | b. ≤5% LMW | 0.0 | 0.0 | 0.0 | 0.0 | 0.0 | 0.0 | 0.0 | 0.0 |
| | species | | | | | | | | |
| | c. ≤7% HMW | 2.2 | 2.2 | 2.2 | 2.1 | 2.3 | 2.3 | 2.5 | 2.6 |
| | species | | | | | | | | |
| Charge | Report % | 39.4 | | | 41.3 | | | | 43.6 |
| Variant | Region 1 | | | | | | | | |
| Analysis | Report % | 42.5 | | | 42.1 | | | | 40.2 |
| by iCIEF | Region 2 | | | | | | | | |
| | Report % | 18.1 | | | 16.6 | | | | 16.3 |
| | Region 3 | | | | | | | | |
| Charge | a. 15-60% | 45.8 | 45.9 | 45.4 | 45.4 | 45.3 | 45.5 | 44.1 | 43.5 |
| Variant | Region 1 | | | | | | | | |
| Analysis | b. % Glycated | 32.5 | 32.6 | 32.3 | 32.1 | 32.3 | 32.3 | 31.7 | 31.2 |
| by CEX | species | | | | | | | | |
| | (report %) | | | | | | | | |
| | c. ≥35% | 48.2 | 48.4 | 48.3 | 48.6 | 48.9 | 48.8 | 49.9 | 50.7 |
| | Region 2 | | | | | | | | |
| | d. ≤25% | 6.0 | 5.7 | 6.3 | 6.0 | 5.8 | 5.7 | 6.1 | 5.8 |
| | Region 3 | | | | | | | | |
| Particulate | Report | 9537 | | | 284 | | | | 298 |
| Matter | particle/mL: | | | | | | | | |
| (MFI ™) | 2 μm ≤ | | | | | | | | |
| | x < 10 μm | | | | | | | | |
| | (particle/mL) | | | | | | | | |
| | ≥10 μm | 622 | | | 35 | | | | 29 |
| | (particle/mL) | | | | | | | | |
| | ≥25 μm | 76 | | | 17 | | | | 2 |
| | (particle/mL) | | | | | | | | |

^{a}Criteria adopted from FBP-015-FD FDG platform Quality Target with program specific adjustment. Results from the assays without Quality Target reported percentage for information only. Boxes greyed to indicate tests not being performed at set time point.
CEX, cation exchange;
DS, drug substance;
FDG, formulation development group;
HMW, high molecular weight;
iCIEF, imaged capillary isoelectric focusing;
LEFVP, liquid essential free of visible particles;
LMW, low molecular weight;
MFI, Micro-Flow Imaging ™;
MP, main peak;
SE-UPLC, size-exclusion ultra high-performance liquid chromatography.

TABLE 51B
Stability of mAb1 Formulated Drug Substance Incubated at 25° C./60% RH and 40° C./75% RH

| | |
|---|---|
| Formulation | 150 mg/mL REGN4018, 30 mM acetate, pH 5.0, 8% (w/v) sucrose, 0.05% |
| | polysorbate 20 |
| Fill Volume | 2.5 mL |
| Storage Container | 5 mL Cellon polycarbonate vial with HDPE lined closure |

| | | Storage Condition | | | | | | | | |
|---|---|---|---|---|---|---|---|---|---|---|
| | Quality | No | 25° C./60% RH Storage | | | | 40° C./75% RH Storage | | | |
| | Acceptance | Storage | (months) | | | | (months) | | | |
| Assay | Criteria^{a} | T = 0 | 0.5 | 1 | 3 | 6 | 0.25 | 0.5 | 1 | 3 |
| Physical | Liquid | LEFVP | LEFVP | LEFVP | LEFVP | LEFVP | LEFVP | LEFVP | LEFVP | LEFVP |
| Form/ | essentially | | | | | | | | | |
| Condition | free from | | | | | | | | | |
| | visible | | | | | | | | | |
| | particulates | | | | | | | | | |
| Clarity | Not more | Not > II | Not > II | Not > III | Not > III | Not > III | Not > II | Not > III | Not > III | Not > III |
| | turbid than | | | | | | | | | |
| | reference | | | | | | | | | |
| | suspension IV | | | | | | | | | |
| Color | Not more | Not > | Not > | Not > | Not > | Not > | Not > | Not > | Not > | Not > |
| | intensely | BY4 | BY4 | BY4 | BY4 | BY4 | BY4 | BY4 | BY4 | BY3 |
| | colored than | | | | | | | | | |
| | reference | | | | | | | | | |
| | solution BY2 | | | | | | | | | |
| pH | 4.8 to 5.2 | 5.0 | 5.0 | 5.0 | 5.1 | 5.0 | 5.0 | 5.0 | 5.0 | 5.0 |
| Total | 45 to 55 | 146.3 | 149.2 | 149.5 | 160.6 | 162.6 | 142.5 | 146.8 | 151.9 | 164.0 |
| Protein | mg/mL | | | | | | | | | |
| Purity by | a. Purity ≥90% | 97.8 | 97.4 | 97.1 | 96.5 | 95.7 | 95.7 | 94.7 | 92.1 | 80.9 |
| SE-UPLC | total peak area | | | | | | | | | |
| | b. ≤5% LMW | 0.0 | 0.0 | 0.1 | 0.0 | 0.1 | 0.3 | 0.0 | 0.1 | 1.2 |
| | species | | | | | | | | | |
| | c. ≤7% HMW | 2.2 | 2.6 | 2.8 | 3.5 | 4.2 | 4.0 | 5.3 | 7.8 | 17.9 |
| | species | | | | | | | | | |
| Charge | Report % | 39.4 | | | | 50.5 | | | | 70.1 |
| Variant | Region 1 | | | | | | | | | |
| Analysis | Report % | 42.5 | | | | 32.3 | | | | 19.5 |
| by iCIEF | Region 2 | | | | | | | | | |
| | Report % | 18.1 | | | | 17.2 | | | | 10.3 |
| | Region 3 | | | | | | | | | |
| Charge | a. 15-60% | 45.8 | 41.4 | 38.7 | 29.2 | 23.4 | 30.9 | 23.5 | 18.4 | 25.2 |
| Variant | Region 1 | | | | | | | | | |
| Analysis | b. % Glycated | 32.5 | 29.5 | 27.2 | 18.6 | 13.1 | 20.4 | 13.4 | 7.6 | 12.0 |
| by CEX | species | | | | | | | | | |
| | (report %) | | | | | | | | | |
| | c. ≥35% | 48.2 | 52.8 | 56.0 | 65.5 | 70.5 | 63.4 | 69.2 | 72.0 | 55.0 |
| | Region 2 | | | | | | | | | |
| | d. ≤25% | 6.0 | 5.8 | 5.3 | 5.3 | 6.1 | 5.7 | 7.3 | 9.6 | 19.8 |
| | Region 3 | | | | | | | | | |
| Particulate | Report | 9537 | | | | 58 | | | | 113 |
| Matter | particle/mL: | | | | | | | | | |
| (MFI ™) | 2 μm ≤ | | | | | | | | | |
| | x < 10 μm | | | | | | | | | |
| | (particle/mL) | | | | | | | | | |
| | ≥10 μm | 622 | | | | 4 | | | | 19 |
| | (particle/mL) | | | | | | | | | |
| | ≥25 μm | 76 | | | | 4 | | | | 0 |
| | (particle/mL) | | | | | | | | | |

^{a}Criteria adopted from FBP-015-FD FDG platform Quality Target with program specific adjustment. Results from the assays without Quality Target reported percentage for information only. Boxes greyed to indicate tests not being performed at set time point.
CEX, cation exchange;
DS, drug substance;
FDG, formulation development group;
HMW, high molecular weight;
iCIEF, imaged capillary isoelectric focusing;
LEFVP, liquid essential free of visible particles;
LMW, low molecular weight;
MFI, Micro-Flow Imaging ™;
MP, main peak;
SE-UPLC, size-exclusion ultra high-performance liquid chromatography.

TABLE 52
Stability of mAb1 Formulated Drug Substance - Effect of Agitation and Freeze/Thaw

| | |
|---|---|
| Formulation | 150 mg/mL REGN4018, 30 mM acetate, pH 5.0, 8% (w/v) sucrose, 0.05% |
| | polysorbate 20 |
| Fill Volume | 2.5 mL |
| Storage Container | 5 mL Cellon polycarbonate vial with HDPE lined closure |

| | | Storage Condition | | | | |
|---|---|---|---|---|---|---|
| | Quality | | Agitation | | Freeze/Thaw | |
| | Acceptance | No Stress | (minutes) | | (cycles) | |
| Assay | Criteria^{a} | T = 0 | 60 | 120 | 2 | 4 |
| Physical | Liquid | LEFVP | LEFVP | LEFVP | LEFVP | LEFVP |
| Form/ | essentially | | | | | |
| Condition | free from | | | | | |
| | visible | | | | | |
| | particulates | | | | | |
| Clarity | Not more | Not > II | Not > II | Not > II | Not > II | Not > II |
| | turbid than | | | | | |
| | reference | | | | | |
| | suspension IV | | | | | |
| Color | Not more | Not > BY4 | Not > BY4 | Not > BY4 | Not > BY4 | Not > BY4 |
| | intensely | | | | | |
| | colored than | | | | | |
| | reference | | | | | |
| | solution BY2 | | | | | |
| pH | 5.8 to 6.2 | 5.0 | 5.0 | 5.0 | 5.0 | 5.0 |
| Total | 22.5 to 27.5 | 146.3 | 145.4 | 144.9 | 147.6 | 146.9 |
| Protein | mg/mL | | | | | |
| Purity by | a. Purity ≥90% | 97.8 | 97.8 | 97.8 | 97.8 | 97.8 |
| SE-UPLC | total peak area | | | | | |
| | b. ≤5% LMW | 0.0 | 0.0 | 0.0 | 0.0 | 0.0 |
| | species | | | | | |
| | c. ≤7% HMW | 2.2 | 2.2 | 2.2 | 2.2 | 2.2 |
| | species | | | | | |
| Charge | a. ≤75% | 39.4 | | 39.3 | | 39.3 |
| Variant | Region 1 | | | | | |
| Analysis | b. ≥25% | 42.5 | | 43.0 | | 43.2 |
| by iCIEF | Region 2 | | | | | |
| | c. ≤25% | 18.1 | | 17.7 | | 17.6 |
| | Region 3 | | | | | |
| Charge | a. 15-60% | 45.8 | 45.6 | 45.8 | 45.3 | 45.5 |
| Variant | Region 1 | | | | | |
| Analysis | b. % Glycated | 32.5 | 32.4 | 32.4 | 32.3 | 32.5 |
| by CEX | species | | | | | |
| | (report %) | | | | | |
| | c. ≥35% | 48.2 | 48.3 | 48.3 | 48.5 | 48.3 |
| | Region 2 | | | | | |
| | d. ≤25% | 6.0 | 6.0 | 5.9 | 6.2 | 6.2 |
| | Region 3 | | | | | |
| Particulate | 2 μm ≤ | 9537 | | 1353 | | 242 |
| Matter | x < 10 μm | | | | | |
| (MFI ™) | (particle/mL) | | | | | |
| | ≥10 μm | 622 | | 90 | | 19 |
| | (particle/mL) | | | | | |
| | ≥25 μm | 76 | | 15 | | 13 |
| | (particle/mL) | | | | | |

^{a}Criteria adopted from FBP-015-FD FDG platform Quality Target with program specific adjustment. Results from the assays without Quality Target reported percentage for information only. Boxes greyed to indicate tests not being performed at set time point.
CEX, cation exchange;
DS, drug substance;
FDG, formulation development group;
HMW, high molecular weight;
iCIEF, imaged capillary isoelectric focusing;
LEFVP, liquid essential free of visible particles;
LMW, low molecular weight;
Micro-Flow Imaging ™;
MP, main peak;
SE-UPLC, size-exclusion ultra high-performance liquid chromatography.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A stable liquid pharmaceutical formulation comprising:
(a) a bispecific antibody at a concentration of from 1 mg/ml±0.1 mg/ml to 200 mg/ml±20 mg/ml, wherein the bispecific antibody comprises a first antigen-binding domain that binds specifically to human MUC16 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15;
(b) a buffer comprising sodium acetate at a concentration of from 10 mM±1 mM to 50 mM±5 mM;
(c) an organic co-solvent comprising polysorbate at a concentration of from 0.01%±0.005% to 0.5%±0.05% w/v; and
(d) a stabilizer comprising a sucrose at a concentration of from 5%±1% to 20%±4% w/v;
wherein the formulation has a pH of 5.0±0.5.

2. (canceled)

3. The pharmaceutical formulation of claim 1, wherein the antibody concentration is from 5 mg/ml±0.5 mg/ml to 50 mg/ml±5 mg/ml, or 5 mg/ml±0.5 mg/ml, or 50 mg/ml±5 mg/ml.

4. The pharmaceutical formulation of claim 1, wherein the antibody concentration is 125 mg/ml to 175 mg/ml, or 150 mg/ml±10 mg/ml.

5-6. (canceled)

7. The pharmaceutical formulation of claim 1, wherein the acetate buffer concentration is from 25 mM±2.5 mM to 35 mM±3.5 mM, or 30 mM±3 mM.

8-9. (canceled)

10. The pharmaceutical formulation of claim 3, wherein the polysorbate concentration is from 0.1%±0.05% to 0.3%±0.03% w/v, or 0.2%±0.02% w/v.

11. The pharmaceutical formulation of claim 4, wherein the polysorbate concentration is from 0.01% w/v to 0.1% w/v, or 0.05% w/v±0.01% w/v.

12. The pharmaceutical formulation of claim 1, wherein the polysorbate is polysorbate 20.

13-14. (canceled)

15. The pharmaceutical formulation of claim 3, wherein the sucrose concentration is from 7%±0.5% to 12%±0.5% w/v, or 10%±1% w/v.

16. The pharmaceutical formulation of claim 4, wherein the sucrose concentration is from 4% to 12% w/v, or 8% w/v±1% w/v.

17. The pharmaceutical formulation of claim 1 comprising:
(a) 5 mg/ml±0.5 mg/ml antibody,
(b) from 25 mM±2 mM to 35 mM±2 mM acetate buffer,
(c) from 0.1%±0.05% to 0.3%±0.05% w/v polysorbate, and
(d) from 5%±1% to 15%±3% w/v sucrose,
at pH 5.0±0.5.

18. The pharmaceutical formulation of claim 17 comprising:
(a) 5 mg/ml±0.5 mg/ml antibody,
(b) 30 mM±1 mM acetate buffer,
(c) 0.2%±0.02% w/v polysorbate 20, and
(d) 10%±1% w/v sucrose,
at pH 5.0±0.3.

19. The pharmaceutical formulation of claim 1 comprising:
(a) 50 mg/ml±5 mg/ml antibody,
(b) from 25 mM±2 mM to 35 mM±2 mM acetate buffer,
(c) from 0.1%±0.05% to 0.3%±0.05% w/v polysorbate, and
(d) from 5%±1% to 15%±3% w/v sucrose,
at pH 5.0±0.5.

20. The pharmaceutical formulation of claim 19 comprising:
(a) 50 mg/ml±0.5 mg/ml antibody,
(b) 30 mM±1 mM acetate buffer,
(c) 0.2%±0.02% w/v polysorbate 20, and
(d) 10%±1% w/v sucrose,
at pH 5.0±0.3.

21. (canceled)

22. The pharmaceutical formulation of claim 1, wherein the formulation contains:
(a) no more than 2.5% high molecular weight (HMW) species after 12 months or 24 months of storage at 5° C., as determined by SE-UPLC;
(b) no more than 3.5% high molecular weight (HMW) species after 6 months of storage at 25° C. and 60% relative humidity, as determined by SE-UPLC;
(c) no more than 1.5% high molecular weight (HMW) species after 12 months of storage at −30° C., or no more than 2.0% HMW species after 24 months of storage at −30° C., as determined by SE-UPLC; or
(d) no more than 1.5% high molecular weight (HMW) species after 12 months of storage at −80° C., or no more than 2.0% HMW species after 24 months of storage at −30° C., as determined by SE-UPLC.

23-48. (canceled)

49. The pharmaceutical formulation of claim 1, wherein the formulation contains no more than 40% of a glycated species variant, wherein the glycated species variant comprises glycation at residue 98 of SEQ ID NO: 1 or SEQ ID NO: 4, or residue 2 of SEQ ID NO: 9.

50. The pharmaceutical formulation of claim 1, wherein the first antigen-binding domain comprises a HCVR with at least 90% identity to the amino acid sequence of SEQ ID NO: 4 and a LCVR with at least 90% identity to the amino acid sequence of SEQ ID NO: 6, and the second antigen-binding domain comprises a HCVR with at least 90% identity to the amino acid sequence of SEQ ID NO: 5 and a LCVR with at least 90% identity to the amino acid sequence of SEQ ID NO: 6.

51-52. (canceled)

53. The pharmaceutical formulation of claim 1, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6.

54. A stable pharmaceutical formulation comprising:
(a) 5 mg/ml±0.5 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 30 mM±1 mM sodium acetate buffer, pH 5.0±0.2,
(c) 0.2%±0.02% w/v polysorbate 20, and
(d) 10%±1% w/v sucrose.

55. A stable pharmaceutical formulation comprising:
(a) 50 mg/ml±5 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 30 mM±1 mM sodium acetate buffer, pH 5.0±0.2,
(c) 0.2%±0.02% w/v polysorbate 20, and
(d) 10%±1% w/v sucrose.

56. A stable pharmaceutical formulation comprising:
(a) 150 mg/ml±15 mg/ml of a bispecific antibody comprising a first antigen-binding domain that binds specifically to human MUC16 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 30 mM±1 mM sodium acetate buffer, pH 5.0±0.2,
(c) 0.05%±0.01% w/v polysorbate 20, and
(d) 8%±1% w/v sucrose.

57. The pharmaceutical formulation of claim 53, wherein the antibody comprises a human IgG heavy chain constant region attached, respectively, to the HCVR of each of the first antigen-binding domain and the second antigen-binding domain.

58. The pharmaceutical formulation of claim 57, wherein the heavy chain constant region is of isotype IgG1, or isotype IgG4.

59. (canceled)

60. The pharmaceutical formulation of claim 57, wherein the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification.

61. (canceled)

62. The pharmaceutical formulation of claim 60, wherein the modification comprises a H435R substitution and a Y436F substitution (EU numbering) in a heavy chain of isotype IgG1 or IgG4.

63. The pharmaceutical formulation of claim 57, wherein the antibody comprises a heavy chain constant region comprising an amino acid sequence selected from the group consisting of SEQ ID NO: 16, SEQ ID NO: 17, SEQ ID NO: 18, and SEQ ID NO: 19.

64. The pharmaceutical formulation of claim 63, wherein the antibody comprises a heavy chain constant region comprising the amino acid sequence of SEQ ID NO: 16 and a heavy chain constant region comprising the amino acid sequence of SEQ ID NO: 17.

65. The pharmaceutical formulation of claim 63, wherein the antibody comprises a heavy chain constant region comprising the amino acid sequence of SEQ ID NO: 18 and a heavy chain constant region comprising the amino acid sequence of SEQ ID NO: 19.

66. The pharmaceutical formulation of claim 53, wherein the antibody comprises a first heavy chain containing the HCVR of the first antigen-binding domain and a second heavy chain containing the HCVR of the second antigen-binding domain, wherein the first heavy chain comprises residues 1-442 of the amino acid sequence of SEQ ID NO: 1 and the second heavy chain comprises residues 1-449 of the amino acid sequence of SEQ ID NO: 2.

67. The pharmaceutical formulation of claim 66, wherein the antibody comprises a common light chain containing the LCVR of the first and second antigen-binding domains, wherein the common light chain comprises the amino acid sequence of SEQ ID NO: 3.

68. The pharmaceutical formulation of claim 1, wherein the percentage change in glycated species is: (i) no more than 1.5% after 6 months of storage at 5° C.; (ii) no more than 3% after 12 months of storage at 5° C.; (iii) no more than 1.5% after 12 months, after 18 months, or after 24 months of storage at −30° C.; or no more than 1% after 12 months, after 18 months, or after 24 months of storage at −80° C., as determined by cation exchange ultra performance liquid chromatography (CEX-UPLC), and/or by liquid chromatography-mass spectrometry (LC-MS).

69. A pharmaceutical composition, wherein the composition comprises the pharmaceutical formulation of claim 1, and the composition is contained in a container.

70. The pharmaceutical composition of claim 69, wherein the container is:
(a) a vial, or a 2 ml, 5 ml or 10 ml Type 1 clear glass vial;
(b) a syringe, or a low tungsten glass syringe;
(c) a prefilled syringe; or
(d) an autoinjector.

71-75. (canceled)

76. A kit comprising (i) a container containing a composition comprising the pharmaceutical formulation of claim 1, and instructions for use of the composition.

77. The kit of claim 76, wherein the container is a glass vial, a prefilled syringe, or an autoinjector, and the instructions recited subcutaneous administration of the composition or intravenous administration of the composition.

78-81. (canceled)

82. A unit dosage form comprising the pharmaceutical formulation of claim 1, wherein the antibody is present in an amount of from 0.1 mg to 500 mg, or from 1 to 20 mg, or from 100 to 200 mg, and the unit dosage form is a glass vial, a prefilled syringe, or an autoinjector.

83-87. (canceled)

88. A container containing a composition comprising the pharmaceutical formulation of claim 1, wherein the container is a glass vial, a prefilled syringe, or an autoinjector.

89-91. (canceled)


SEQUENCE LISTING (19 sequences)

SEQ ID NO: 1 - protein, 443 aa, Artificial Sequence
  Synthetic
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 450 aa, Artificial Sequence
  Synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 3 - protein, 215 aa, Artificial Sequence
  Synthetic
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 117 aa, Artificial Sequence
  Synthetic
QVQLVESGGGLVKPGGSLRLSCAASGFTFSNYYMSWVRQAPGKGLEWISYISGRGSTIFYADSVKGRITISRDNAKNSLFLQMNSLRAEDTAVYFCVKDRGGYSPYWGQGTLVTVSS

SEQ ID NO: 5 - protein, 124 aa, Artificial Sequence
  synthetic
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 6 - protein, 108 aa, Artificial Sequence
  Synthetic
DIQMTQSPSSLSASVGDRVTITCRASQSISTYLNWYQQKPGKAPKLLIYTASSLQSGVPSRFSGSGSGTDFTLTISSLQPEDFATYYCQQSYSTPPITFGQGTRLEIK

SEQ ID NO: 7 - protein, 8 aa, Artificial Sequence
  Synthetic
GFTFSNYY

SEQ ID NO: 8 - protein, 8 aa, Artificial Sequence
  Synthetic
ISGRGSTI

SEQ ID NO: 9 - protein, 10 aa, Artificial Sequence
  Synthetic
VKDRGGYSPY

SEQ ID NO: 10 - protein, 8 aa, Artificial Sequence
  synthetic
GFTFDDYS

SEQ ID NO: 11 - protein, 8 aa, Artificial Sequence
  synthetic
ISWNSGSK

SEQ ID NO: 12 - protein, 17 aa, Artificial Sequence
  synthetic
AKYGSGYGKFYHYGLDV

SEQ ID NO: 13 - protein, 6 aa, Artificial Sequence
  Synthetic
QSISTY

SEQ ID NO: 14 - protein, 3 aa, Artificial Sequence
  Synthetic
TAS

SEQ ID NO: 15 - protein, 10 aa, Artificial Sequence
  Synthetic
QQSYSTPPIT

SEQ ID NO: 16 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 326 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 18 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK